(19) United States
(12) Patent Application Publication
(10) Pub. No.: US 2026/0185996 A1
(43) Pub. Date: Jul. 2, 2026

(54) METHODS OF CONTROLLED CLEAVAGE

(71) Applicant: Quantum-Si Incorporated, Branford, CT (US)

(72) Inventors: Haidong Huang, Madison, CT (US); Brandon Choi, Branford, CT (US)

(73) Assignee: Quantum-Si Incorporated, Branford, CT (US)

(21) Appl. No.: 19/301,759

(22) Filed: Aug. 15, 2025

Related U.S. Application Data
(60) Provisional application No. 63/684,315, filed on Aug. 16, 2024.

(51) Int. Cl. G01N 33/68; C12N 9/48

(52) U.S. Cl. CPC G01N 33/6818; C12N 9/485; C12Y 304/19001; G01N 2333/91057; G01N 2333/948

(57) ABSTRACT

Provided herein are methods that are useful for controlled cleavage of polypeptides (e.g., modified polypeptides). In some aspects, provided herein are methods that are useful in combination with polypeptide sequencing techniques. For example, provided herein is a method comprising contacting a terminal amino acid of a polypeptide with one or more modification agents to produce a polypeptide comprising a modified terminal amino acid; and contacting the polypeptide comprising a modified terminal amino acid with an enzyme that catalyzes the removal of the modified terminal amino acid, wherein the enzyme has increased catalytic activity for removal of the modified terminal amino acid relative to a reference polypeptide comprising an unmodified terminal amino acid.

### CROSS-REFERENCE TO RELATED APPLICATIONS

This application claims priority under 35 U.S.C. § 119(e) to U.S. Provisional Application No. 63/684,315, filed Aug. 16, 2024, which is hereby incorporated by reference in its entirety.

### REFERENCE TO AN ELECTRONIC SEQUENCE LISTING

The content of the electronic sequence listing (R070870173US01-SEQ-JIB.xml; Size: 50,658 bytes; and Date of Creation: Aug. 15, 2025) is herein incorporated by reference in its entirety.

### BACKGROUND

Proteins are the main structural and functional components of cells, driving key biological and cellular processes. Next-generation DNA sequencing technologies have revolutionized our understanding of heredity and gene regulation, but the complex and dynamic states of cells are not fully captured by the genome and transcriptome. Applying similar approaches to proteomics has been difficult because of the scale, dynamic range, and inability to amplify the source.

### SUMMARY

Conventional peptide sequencing by Edman degradation requires subjecting peptides to relatively harsh reaction conditions (e.g., low pH, high temperature) over multiple reaction cycles, which can damage peptides or surface attachments and ultimately limit the extent of sequence information obtained. This chemical approach to Edman degradation has been adapted to an enzymatic approach that uses a modified protease capable of cleaving PITC-derivatized amino acids. Although the enzymatic approach involves relatively milder reaction conditions, the enzyme shows low turnover and high reagent specificity (see, e.g., Callahan, et al. Trends Biochem Sci. 2020 January; 45(1):76-89).

Other approaches to peptide sequencing rely on stochastic cleavage mechanisms. While these mechanisms have the advantage of simpler workflows and milder reaction conditions, stochastic cleavage mechanisms can involve a high usage of reagents for every detected amino acid. These approaches utilize kinetic principles which allow for more structural information to be extracted from peptides than previous methods, and in some aspects, the disclosure relates to methods of peptide sequencing that advantageously maintain this high sensitivity and overcome the limitations of prior cleavage mechanisms and reagents.

To address the issues raised by stochastic cleavage mechanisms, the inventors of the present disclosure identified and developed controlled cleavage mechanisms with reduced reagent consumption (e.g., cleavage reagent consumption) that permits the removal of a single amino acid at a given point from a target polypeptide. These controlled cleavage mechanisms, which are provided herein, provide accurate detection of amino acids, and thus determination of sequence information, within a target polypeptide (e.g., by generating specifically defined detection windows). As described further herein, the controlled cleavage mechanisms provided herein surprisingly result in a reduction in undesired amino acid deletions, a reduction in skipping of certain amino acids for detection, and a reduction in excess number of sequence events with different populations as compared to currently utilized stochastic cleavage mechanisms. This provides the research end user enhanced control, in part because the controlled cleavage mechanisms provided herein do not involve the continuous activation and cleavage characteristic of stochastic cleavage mechanisms, which facilitates diagnosis of problems within a sequencing run. Such controlled cleavage mechanisms involve at least two distinct steps—modification (or activation) of a terminal amino acid to produce a modified terminal amino acid for selective removal of that modified terminal amino acid (relative to an unmodified amino acid), followed by the introduction of reaction conditions and/or reagents that result in the removal of the modified terminal amino acid (without removing a subsequent terminal amino acid that remains unmodified).

Accordingly, in some aspects, the present disclosure provides a method for controlled cleavage of a polypeptide (e.g., in a method of polypeptide sequencing).

Some aspects of the disclosure provide a method comprising: (a) contacting a terminal amino acid of a polypeptide with one or more modification agents to produce a polypeptide comprising a modified terminal amino acid; (b) contacting the polypeptide comprising a modified terminal amino acid with an enzyme that catalyzes the removal of the modified terminal amino acid, wherein the enzyme has increased catalytic activity for removal of the modified terminal amino acid relative to a reference polypeptide comprising an unmodified terminal amino acid. In some embodiments, the method further comprises: (c) contacting the polypeptide with a composition comprising one or more terminal amino acid recognition molecules; and (d) detecting a series of signal pulses indicative of association of the one or more terminal amino acid recognition molecules with a terminus of the polypeptide, wherein the series of signal pulses is indicative of the identity of the terminal amino acid. In some embodiments, the composition of (c) does not comprise a cleaving reagent (e.g., an enzyme capable of cleaving terminal amino acids, such as a peptidase).

Other aspects of the disclosure provide a method of polypeptide sequencing, the method comprising: (a) contacting a terminal amino acid of a polypeptide with one or more modification agents to produce a polypeptide comprising a modified terminal amino acid; (b) contacting the polypeptide comprising a modified terminal amino acid with an enzyme that catalyzes the removal of the modified terminal amino acid, wherein the enzyme has increased catalytic activity for removal of the modified terminal amino acid relative to a reference polypeptide comprising an unmodified terminal amino acid; (c) contacting the polypeptide with a composition comprising one or more terminal amino acid recognition molecules; (d) detecting a series of signal pulses indicative of association of the one or more terminal amino acid recognition molecules with a terminus of the polypeptide, wherein the series of signal pulses is indicative of the identity of the terminal amino acid; and (e) repeating steps (a)-(d), thereby sequencing a segment of the polypeptide. In some embodiments, the composition of (c) does not comprise a cleaving reagent (e.g., an enzyme capable of cleaving terminal amino acids, such as a peptidase).

Some aspects of the disclosure provide a method comprising: (a) contacting a terminal amino acid of a polypeptide with one or more acetylation agents to produce a polypeptide comprising a modified terminal amino acid, wherein the modified terminal amino acid is an acetylated terminal amino acid; (b) contacting the polypeptide comprising a modified terminal amino acid with an enzyme that catalyzes the removal of an acetylated amino acid. Other aspects of the disclosure provide a method of polypeptide sequencing, the method comprising: (a) contacting a terminal amino acid of a polypeptide with one or more acetylation agents to produce a polypeptide comprising a modified terminal amino acid, wherein the modified terminal amino acid is an acetylated terminal amino acid; (b) contacting the polypeptide comprising a modified terminal amino acid with an enzyme that catalyzes the removal of an acetylated amino acid; (c) contacting the polypeptide with a composition comprising one or more terminal amino acid recognition molecules; (d) detecting a series of signal pulses indicative of association of the one or more terminal amino acid recognition molecules with a terminus of the polypeptide, wherein the series of signal pulses is indicative of the identity of the terminal amino acid; and (e) repeating steps (a)-(d), thereby sequencing a segment of the polypeptide. In some embodiments, the composition of (c) does not comprise a cleaving reagent (e.g., an enzyme capable of cleaving terminal amino acids, such as a peptidase).

In some embodiments, a terminal amino acid is an N-terminal amino acid or a C-terminal amino acid. In some embodiments, a polypeptide is immobilized to a surface. In some embodiments, a polypeptide is immobilized to the surface through a terminal amino acid, wherein (i) the N-terminal amino acid of the polypeptide is contacted with the one or more modification agents and the C-terminal amino acid is immobilized to the surface (e.g., through the carboxyl group or the side chain of the C-terminal amino acid), or (ii) the C-terminal amino acid of the polypeptide is contacted with the one or more modification agents and the N-terminal amino acid is immobilized to the surface (e.g., through the amino group or the side chain of the N-terminal amino acid). In some embodiments, a polypeptide is immobilized to the surface through a linker, optionally wherein the linker comprises a biomolecule, further optionally wherein the biomolecule is an oligonucleotide.

In some embodiments, an N-terminal amino acid of the polypeptide is contacted with the one or more modification agents. In some embodiments, a C-terminal amino acid of the polypeptide is contacted with the one or more modification agents.

In some embodiments, one or more modification agents comprise a small molecule and/or an enzyme, optionally wherein the one or more modification agents comprise or consist of one or more acetylation agents. In some embodiments, the one or more acetylation agents comprise one or more succinimidyl acetate compounds (e.g., N-Hydroxysulfosuccinimide acetate, N-Hydroxysuccinimide acetate, or sulfosuccinimidyl acetate (Sulfo-NHS acetate)). In some embodiments, one or more modification agents comprise an N-Hydroxysulfosuccinimide or an N-Hydroxysuccinimide. In some embodiments, one or more modification agents is sulfosuccinimidyl acetate (Sulfo-NHS acetate). In some embodiments, one or more modification agents comprise or consist of a compound of any one of Formulae (I), (I-a), (I-b), (II), and (III) described herein. In some embodiments, one or more modification agents comprise: (i) an N-terminal acyltransferase, optionally wherein the N-terminal acyltransferase is N-myristoyltransferase or N-alpha-acetyltransferase; and (ii) an acyl-CoA, optionally wherein the acyl-CoA is acetyl-CoA. In some embodiments, the one or more modification agents do not comprise an isothiocyanate compound (e.g., phenyl isothiocyanate).

In some embodiments, one or more acetylation agents comprise an N-Hydroxysulfosuccinimide acetate, an N-Hydroxysuccinimide acetate, or sulfosuccinimidyl acetate (Sulfo-NHS acetate). In some embodiments, one or more acetylation agents comprise or consist of a compound of any one of Formulae (I), (I-a), (I-b), (II), and (III) described herein. In some embodiments, one or more acetylation agents comprise: (i) an N-terminal acyltransferase, optionally wherein the N-terminal acyltransferase is N-myristoyltransferase or N-alpha-acetyltransferase; and (ii) an acyl-CoA, optionally wherein the acyl-CoA is acetyl-CoA.

In some embodiments, contacting of (a) produces a modified terminal amino acid having a free acetyl group. In some embodiments, a polypeptide comprising a modified terminal amino acid is an acetylated polypeptide. In some embodiments, a modified terminal amino acid is an acetylated terminal amino acid. In some embodiments, an enzyme catalyzes the removal of an acetylated amino acid. In some embodiments, an acetylated amino acid is an acetylated terminal amino acid, optionally an acetylated N-terminal amino acid.

In some embodiments, an enzyme is an acylpeptide hydrolase. In some embodiments, an acylpeptide hydrolase is a wild-type acylpeptide hydrolase or an engineered acylpeptide hydrolase. In some embodiments, an acylpeptide hydrolase is a human acylpeptide hydrolase, Sus scrofa acylpeptide hydrolase, Aeropyrum pernix acylpeptide hydrolase, Bombyx mori acylpeptide hydrolase, or Rattus norvegicus acylpeptide hydrolase; and/or the acylpeptide hydrolase comprises an amino acid sequence that is at least 80%, at least 85%, at least 90%, at least 95%, at least 97%, 80-90%, 85-95%, 90-100%, 95-100%, 98-100%, or 100% identical to the amino acid sequence of any one of SEQ ID NOs: 1-25.

In some embodiments, a terminal amino acid is a serine, methionine, leucine or alanine.

In some embodiments, one or more modification agents comprise a handle peptide. In some embodiments, a handle peptide is a single amino acid, a dipeptide, or a tripeptide. In some embodiments, a handle peptide is an acetylated peptide. In some embodiments, a handle peptide is ligated to the terminal amino acid of the polypeptide using chemical synthesis methods. In some embodiments, a handle peptide is ligated to the terminal amino acid of the polypeptide using subtiligase-catalyzed peptide ligation.

In some embodiments, an enzyme is a peptidase, optionally a dipeptidyl-peptidase or a tripeptidyl-peptidase. In some embodiments, a polypeptide comprising a modified terminal amino acid is contacted with a deacetylase prior to contacting with the peptidase. In some embodiments, a polypeptide is immobilized to a surface. In some embodiments, a polypeptide is immobilized to the surface through a terminal amino acid, wherein (i) the N-terminal amino acid of the polypeptide is contacted with the one or more modification agents and the C-terminal amino acid is immobilized to the surface (e.g., through the carboxyl group or the side chain of the C-terminal amino acid), or (ii) the C-terminal amino acid of the polypeptide is contacted with the one or more modification agents and the N-terminal amino acid is immobilized to the surface (e.g., through the amino group or the side chain of the N-terminal amino acid). In some embodiments, a polypeptide is immobilized to the surface through a linker, optionally wherein the linker comprises a biomolecule, further optionally wherein the biomolecule is an oligonucleotide.

In some embodiments, an enzyme that catalyzes the removal of an acetylated amino acid has increased catalytic activity for removal of the acetylated terminal amino acid relative to a reference polypeptide comprising an unmodified terminal amino acid.

In some embodiments of a method of polypeptide sequencing, the method comprises an association of the one or more terminal amino acid recognition molecules with a particular type of terminal amino acid, which produces a characteristic pattern in the series of signal pulses that is different from other types of amino acids. In some embodiments, a characteristic pattern comprises a portion of the series of signal pulses. In some embodiments, a signal pulse of the characteristic pattern corresponds to an individual association event between a terminal amino acid recognition molecule and a terminal amino acid. In some embodiments, a signal pulse of the characteristic pattern comprises a pulse duration that is characteristic of a dissociation rate of binding between the terminal amino acid recognition molecule and the terminal amino acid. In some embodiments, each signal pulse of the characteristic pattern is separated from another by an interpulse duration that is characteristic of an association rate of terminal amino acid recognition molecule binding. In some embodiments, the characteristic pattern corresponds to a series of reversible terminal amino acid recognition molecule binding interactions with the terminal amino acid. In some embodiments, the series of reversible terminal amino acid recognition molecule binding interactions comprises a reversible formation of one binary complex species at the terminus of the polypeptide. In some embodiments, the series of reversible terminal amino acid recognition molecule binding interactions comprises a reversible formation of different binary complex species at the terminus of the polypeptide.

In some embodiments, a method further comprises a washing step between (a) and (b), optionally wherein the washing step comprises washing the polypeptide with a solution to remove excess modification agent(s) or excess acetylation agent(s).

In some aspects, the disclosure provides a method of polypeptide analysis, the method comprising: (a) contacting a polypeptide with one or more acetylation agents to produce an acetylated first amino acid at a terminus of the polypeptide; (b) removing the acetylated first amino acid to expose a second amino acid at the terminus of the polypeptide; (c) contacting the polypeptide having the second amino acid at the terminus with a composition comprising one or more terminal amino acid recognition molecules; and (d) detecting a series of signal pulses corresponding to binding interactions between the one or more terminal amino acid recognition molecules and the terminus of the polypeptide, wherein the series of signal pulses is indicative of the second amino acid.

In some embodiments, the polypeptide is immobilized to a surface. In some embodiments, the polypeptide is immobilized to the surface through a terminal amino acid, wherein (i) the N-terminal amino acid of the polypeptide is contacted with the one or more acetylation agents and the C-terminal amino acid is immobilized to the surface, or (ii) the C-terminal amino acid of the polypeptide is contacted with the one or more acetylation agents and the N-terminal amino acid is immobilized to the surface. In some embodiments, the polypeptide is immobilized to the surface through a linker, optionally wherein the linker comprises a biomolecule, further optionally wherein the biomolecule is an oligonucleotide.

In some embodiments, the one or more acetylation agents comprise one or more succinimidyl acetate compounds. In some embodiments, the one or more succinimidyl acetate compounds comprise an N-Hydroxysulfosuccinimide acetate, an N-Hydroxysuccinimide acetate, or sulfosuccinimidyl acetate (Sulfo-NHS acetate). In some embodiments, the one or more acetylation agents comprise or consist of a compound of any one of Formulae (I), (I-a), (I-b), (II), and (III) described herein.

In some embodiments, the one or more acetylation agents comprise: (i) an N-terminal acyltransferase; and (ii) an acyl-CoA. In some embodiments, the N-terminal acyltransferase is N-myristoyltransferase or N-alpha-acetyltransferase. In some embodiments, the acyl-CoA is acetyl-CoA. In some embodiments, the one or more acetylation agents comprise: (i) N-myristoyltransferase or N-alpha-acetyltransferase; and (ii) acetyl-CoA.

In some embodiments, removing the acetylated first amino acid to expose a second amino acid at the terminus of the polypeptide comprises contacting the polypeptide with an enzyme that catalyzes the removal of an acetylated amino acid. In some embodiments, the enzyme that catalyzes the removal of an acetylated amino acid is an acylpeptide hydrolase (e.g., an acylpeptide hydrolase described herein). In some embodiments, the acylpeptide hydrolase is a wild-type acylpeptide hydrolase or an engineered acylpeptide hydrolase. In some embodiments, the acylpeptide hydrolase is a human acylpeptide hydrolase, Sus scrofa acylpeptide hydrolase, Aeropyrum pernix acylpeptide hydrolase, Bombyx mori acylpeptide hydrolase, or Rattus norvegicus acylpeptide hydrolase. In some embodiments, the acylpeptide hydrolase comprises an amino acid sequence that is at least 80%, at least 85%, at least 90%, at least 95%, at least 97%, 80-90%, 85-95%, 90-100%, 95-100%, 98-100%, or 100% identical to the amino acid sequence of any one of SEQ ID NOs: 1-25. In some embodiments, the acylpeptide hydrolase comprises the amino acid sequence of any one of SEQ ID NOs: 13-25. In some embodiments, the acylpeptide hydrolase consists of the amino acid sequence of any one of SEQ ID NOs: 13-25.

In some embodiments, the method further comprises, between (a) and (b): washing the polypeptide with a solution that does not comprise the one or more acetylation agents. In some embodiments, the washing step comprises washing the polypeptide to remove excess acetylation agent(s). In some embodiments, the method further comprises, between (b) and (c): washing the polypeptide with a solution that does not comprise the enzyme. In some embodiments, the composition of (c) does not comprise a cleaving reagent.

In some embodiments, binding interactions between the one or more terminal amino acid recognition molecules with a particular type of terminal amino acid produces a characteristic pattern in the series of signal pulses that is different from other types of amino acids. In some embodiments, the characteristic pattern comprises a portion of the series of signal pulses. In some embodiments, a signal pulse of the characteristic pattern corresponds to an individual association event between a terminal amino acid recognition molecule and a terminal amino acid. In some embodiments, a signal pulse of the characteristic pattern comprises a pulse duration that is characteristic of a dissociation rate of binding between the terminal amino acid recognition molecule and the terminal amino acid. In some embodiments, each signal pulse of the characteristic pattern is separated from another by an interpulse duration that is characteristic of an association rate of terminal amino acid recognition molecule binding.

In some embodiments, the characteristic pattern corresponds to a series of reversible terminal amino acid recognition molecule binding interactions with the terminal amino acid. In some embodiments, the series of reversible terminal amino acid recognition molecule binding interactions comprises a reversible formation of one binary complex species at the terminus of the polypeptide. In some embodiments, the series of reversible terminal amino acid recognition molecule binding interactions comprises a reversible formation of different binary complex species at the terminus of the polypeptide.

In some embodiments, the method further comprises, prior to (a): (i) contacting the polypeptide with one or more post-translational modification (PTM)-specific affinity reagents; and (ii) detecting a series of signal pulses corresponding to binding interactions between the one or more PTM-specific affinity reagents and the polypeptide, wherein the series of signal pulses is indicative of at least one PTM in the polypeptide. In some embodiments, the method further comprises: (iii) contacting the polypeptide with a composition comprising one or more terminal amino acid recognition molecules; and (iv) detecting a series of signal pulses corresponding to binding interactions between the one or more terminal amino acid recognition molecules and the terminus of the polypeptide, wherein the series of signal pulses is indicative of the first amino acid, optionally wherein, between (ii) and (iii), the polypeptide is washed with a solution to remove excess PTM-specific affinity reagent(s).

In some embodiments, the one or more PTM-specific affinity reagents are antibodies or aptamers. In some embodiments, the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination. In some embodiments, the one or more PTM-specific affinity reagents specifically bind to phospho-tyrosine, phospho-serine, or phospho-threonine. In some embodiments, each PTM-specific affinity reagent comprises a label, optionally wherein the label is a luminescent label.

In some embodiments, the polypeptide is contacted with two or more PTM-specific affinity reagents at the same time. In some embodiments, each of the two or more PTM-specific affinity reagents comprise a unique label relative to the other PTM-specific affinity reagents. In some embodiments, the polypeptide is contacted in series with a first PTM-specific affinity reagent and a second PTM-specific affinity reagent, optionally wherein the first PTM-specific affinity reagent is removed prior to addition of the second PTM-specific affinity reagent.

In some embodiments, binding interactions between the one or more PTM-specific affinity reagents and one type of PTM of the polypeptide produces a characteristic pattern in the series of signal pulses that is different from other types of PTMs. In some embodiments, the characteristic pattern comprises a portion of the series of signal pulses. In some embodiments, the characteristic pattern allows for a determination of the type of amino acids located at positions in proximity to the PTM of the polypeptide. In some embodiments, the characteristic pattern allows for a determination of the location of the PTM within the polypeptide.

In some aspects, the disclosure provides a method of polypeptide analysis, the method comprising: (a) contacting a polypeptide with one or more PTM-specific affinity reagents; (b) detecting a series of signal pulses corresponding to binding interactions between the one or more PTM-specific affinity reagents and the polypeptide, wherein the series of signal pulses is indicative of at least one PTM in the polypeptide; (c) contacting the polypeptide with a composition comprising one or more terminal amino acid recognition molecules; (d) detecting a series of signal pulses corresponding to binding interactions between the one or more terminal amino acid recognition molecules and a terminus of the polypeptide, wherein the series of signal pulses is indicative of a first amino acid at the terminus (e.g., an N-terminal amino acid); (e) contacting the polypeptide with one or more modification agents to produce a modified first amino acid at the terminus of the polypeptide; and (f) removing the modified first amino acid to expose a second amino acid at the terminus of the polypeptide.

In some embodiments, the one or more modification agents comprise one or more acetylation agents described herein, and the modified first amino acid comprises an acetylated first amino acid. In some embodiments, the removing comprises contacting the polypeptide with an acylpeptide hydrolase (e.g., an acylpeptide hydrolase described herein). In some embodiments, the method further comprises: repeating (c)-(f) one or more times. In some embodiments, repeating (c)-(f) determines the identity of one or more amino acids of the polypeptide. In some embodiments, repeating (c)-(f) sequences the polypeptide (e.g., determines an amino acid sequence of at least a portion of the polypeptide).

In some aspects, the disclosure provides an acylpeptide hydrolase comprising an amino acid sequence that is at least 85% identical to SEQ ID NO: 1, wherein the amino acid sequence has one or more substitutions, insertions, and/or deletions relative to SEQ ID NO: 1. In some embodiments, the amino acid sequence comprises a deletion of at least 5 amino acids between positions 2-60, inclusive, relative to SEQ ID NO: 1. In some embodiments, at least a portion of the amino acid sequence is at least 85% identical to positions 61-582 of SEQ ID NO: 1. In some embodiments, the amino acid sequence comprises a deletion of positions 2-15, 2-21, 2-26, 2-30, or 2-53 relative to SEQ ID NO: 1. In some embodiments, the amino acid sequence is at least 85%, at least 90%, at least 95%, at least 99%, or 100% identical to the amino acid sequence of any one of SEQ ID NOs: 8 and 13-16.

In some embodiments, the amino acid sequence comprises an amino acid substitution at one or more positions corresponding to R113, V471, F485, F488, L492, and T527 of SEQ ID NO: 1. In some embodiments, the amino acid substitution at R113 is R113Y or R113N. In some embodiments, the amino acid substitution at V471 is V471T, V471S, or V471I. In some embodiments, the amino acid substitution at F485 is F485L or F485M. In some embodiments, the amino acid substitution at F488 is F488V, F488M, or F488L. In some embodiments, the amino acid substitution at L492 is L492I. In some embodiments, the amino acid substitution at T527 is T527S.

In some embodiments, the amino acid sequence comprises an F485L substitution relative to SEQ ID NO: 1. In some embodiments, the amino acid sequence is at least 85%, at least 90%, at least 95%, at least 99%, or 100% identical to the amino acid sequence set forth in SEQ ID NO: 17.

In some embodiments, the amino acid sequence comprises F485L and F488V substitutions relative to SEQ ID NO: 1. In some embodiments, the amino acid sequence is at least 85%, at least 90%, at least 95%, at least 99%, or 100% identical to the amino acid sequence set forth in SEQ ID NO: 18.

In some embodiments, the amino acid sequence comprises F485L and F488M substitutions relative to SEQ ID NO: 1. In some embodiments, the amino acid sequence is at least 85%, at least 90%, at least 95%, at least 99%, or 100% identical to the amino acid sequence set forth in SEQ ID NO: 19.

In some embodiments, the amino acid sequence comprises F485L and L492I substitutions relative to SEQ ID NO: 1. In some embodiments, the amino acid sequence is at least 85%, at least 90%, at least 95%, at least 99%, or 100% identical to the amino acid sequence set forth in SEQ ID NO: 20.

In some embodiments, the amino acid sequence comprises R113Y, F485L, and F488L substitutions relative to SEQ ID NO: 1. In some embodiments, the amino acid sequence is at least 85%, at least 90%, at least 95%, at least 99%, or 100% identical to the amino acid sequence set forth in SEQ ID NO: 21.

In some embodiments, the amino acid sequence comprises R113N, F485M, F488L substitutions relative to SEQ ID NO: 1. In some embodiments, the amino acid sequence is at least 85%, at least 90%, at least 95%, at least 99%, or 100% identical to the amino acid sequence set forth in SEQ ID NO: 22.

In some embodiments, the amino acid sequence comprises a V471T substitution relative to SEQ ID NO: 1. In some embodiments, the amino acid sequence is at least 85%, at least 90%, at least 95%, at least 99%, or 100% identical to the amino acid sequence set forth in SEQ ID NO: 23.

In some embodiments, the amino acid sequence comprises a V471S substitution relative to SEQ ID NO: 1. In some embodiments, the amino acid sequence is at least 85%, at least 90%, at least 95%, at least 99%, or 100% identical to the amino acid sequence set forth in SEQ ID NO: 24.

In some embodiments, the amino acid sequence comprises V471I, F485L, F488L, and T527S substitutions relative to SEQ ID NO: 1. In some embodiments, the amino acid sequence is at least 85%, at least 90%, at least 95%, at least 99%, or 100% identical to the amino acid sequence set forth in SEQ ID NO: 25.

In some embodiments, the amino acid sequence comprises amino acid substitutions at two or more positions corresponding to R113, V471, F485, F488, L492, and T527 of SEQ ID NO: 1. In some embodiments, the amino acid sequence comprises a deletion of positions 2-21 relative to SEQ ID NO: 1.

In some aspects, this disclosure provides a method of polypeptide analysis, the method comprising: (a) contacting a polypeptide with one or more acetylation agents to produce an acetylated first amino acid at a terminus of the polypeptide; (b) contacting the polypeptide with an acylpeptide hydrolase of the present disclosure to remove the acetylated first amino acid and expose a second amino acid at the terminus of the polypeptide. In some embodiments, the method further comprises: (c) contacting the polypeptide having the second amino acid at the terminus with a composition comprising one or more terminal amino acid recognition molecules; and (d) detecting a series of signal pulses corresponding to binding interactions between the one or more terminal amino acid recognition molecules and the terminus of the polypeptide, wherein the series of signal pulses is indicative of the second amino acid.

In some embodiments, the polypeptide is immobilized to a surface. In some embodiments, the polypeptide is immobilized to the surface through a terminal amino acid, wherein (i) the N-terminal amino acid of the polypeptide is contacted with the one or more acetylation agents and the C-terminal amino acid is immobilized to the surface, or (ii) the C-terminal amino acid of the polypeptide is contacted with the one or more acetylation agents and the N-terminal amino acid is immobilized to the surface. In some embodiments, the polypeptide is immobilized to the surface through a linker, optionally wherein the linker comprises a biomolecule, further optionally wherein the biomolecule is an oligonucleotide.

In some embodiments, the one or more acetylation agents comprise one or more succinimidyl acetate compounds, optionally wherein the one or more succinimidyl acetate compounds comprises an N-Hydroxysulfosuccinimide acetate, an N-Hydroxysuccinimide acetate, or sulfosuccinimidyl acetate (Sulfo-NHS acetate). In some embodiments, the one or more acetylation agents comprise or consist of a compound of any one of Formulae (I), (I-a), (I-b), (II), and (III) described herein. In some embodiments, the one or more acetylation agents comprise: (i) an N-terminal acyltransferase, optionally wherein the N-terminal acyltransferase is N-myristoyltransferase or N-alpha-acetyltransferase; and (ii) an acyl-CoA, optionally wherein the acyl-CoA is acetyl-CoA.

In some embodiments, the method further comprises, between (a) and (b): washing the polypeptide with a solution that does not comprise the one or more acetylation agents. In some embodiments, the washing step comprises washing the polypeptide to remove excess acetylation agent(s). In some embodiments, the method further comprises, between (b) and (c): washing the polypeptide with a solution that does not comprise the acylpeptide hydrolase. In some embodiments, the composition of (c) does not comprise a cleaving reagent.

In some embodiments, binding interactions between the one or more terminal amino acid recognition molecules with a particular type of terminal amino acid produces a characteristic pattern in the series of signal pulses that is different from other types of amino acids. In some embodiments, the characteristic pattern comprises a portion of the series of signal pulses. In some embodiments, a signal pulse of the characteristic pattern corresponds to an individual association event between a terminal amino acid recognition molecule and a terminal amino acid. In some embodiments, a signal pulse of the characteristic pattern comprises a pulse duration that is characteristic of a dissociation rate of binding between the terminal amino acid recognition molecule and the terminal amino acid. In some embodiments, each signal pulse of the characteristic pattern is separated from another by an interpulse duration that is characteristic of an association rate of terminal amino acid recognition molecule binding.

In some embodiments, the characteristic pattern corresponds to a series of reversible terminal amino acid recognition molecule binding interactions with the terminal amino acid. In some embodiments, the series of reversible terminal amino acid recognition molecule binding interactions comprises a reversible formation of one binary complex species at the terminus of the polypeptide. In some embodiments, the series of reversible terminal amino acid recognition molecule binding interactions comprises a reversible formation of different binary complex species at the terminus of the polypeptide.

In some embodiments, the method further comprises, prior to (a): (i) contacting the polypeptide with one or more post-translational modification (PTM)-specific affinity reagents; and (ii) detecting a series of signal pulses corresponding to binding interactions between the one or more PTM-specific affinity reagents and the polypeptide, wherein the series of signal pulses is indicative of at least one PTM in the polypeptide. In some embodiments, the method further comprises: (iii) contacting the polypeptide with a composition comprising one or more terminal amino acid recognition molecules; and (iv) detecting a series of signal pulses corresponding to binding interactions between the one or more terminal amino acid recognition molecules and the terminus of the polypeptide, wherein the series of signal pulses is indicative of the first amino acid, optionally wherein, between (ii) and (iii), the polypeptide is washed with a solution to remove excess PTM-specific affinity reagent(s).

In some embodiments, the one or more PTM-specific affinity reagents are antibodies or aptamers. In some embodiments, the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination. In some embodiments, the one or more PTM-specific affinity reagents specifically bind to phospho-tyrosine, phospho-serine, or phospho-threonine. In some embodiments, each PTM-specific affinity reagent comprises a label, optionally wherein the label is a luminescent label.

In some embodiments, the polypeptide is contacted with two or more PTM-specific affinity reagents at the same time. In some embodiments, each of the two or more PTM-specific affinity reagents comprise a unique label relative to the other PTM-specific affinity reagents. In some embodiments, the polypeptide is contacted in series with a first PTM-specific affinity reagent and a second PTM-specific affinity reagent, optionally wherein the first PTM-specific affinity reagent is removed prior to addition of the second PTM-specific affinity reagent.

In some embodiments, binding interactions between the one or more PTM-specific affinity reagents and one type of PTM of the polypeptide produces a characteristic pattern in the series of signal pulses that is different from other types of PTMs. In some embodiments, the characteristic pattern comprises a portion of the series of signal pulses. In some embodiments, the characteristic pattern allows for a determination of the type of amino acids located at positions in proximity to the PTM of the polypeptide. In some embodiments, the characteristic pattern allows for a determination of the location of the PTM within the polypeptide.

In some aspects, the disclosure provides a kit comprising: one or more modification agents described herein; and one or more enzymes described herein.

In some aspects, the disclosure provides a kit comprising: one or more acetylation agents; and one or more acylpeptide hydrolases.

In some embodiments, the one or more acetylation agents comprise one or more succinimidyl acetate compounds. In some embodiments, the one or more succinimidyl acetate compounds are selected from N-hydroxysulfosuccinimide acetate, N-hydroxysuccinimide acetate, and sulfosuccinimidyl acetate (Sulfo-NHS acetate). In some embodiments, the one or more succinimidyl acetate compounds comprise Sulfo-NHS acetate. In some embodiments, the one or more acetylation agents comprise or consist of a compound of any one of Formulae (I), (I-a), (I-b), (II), and (III) described herein.

In some embodiments, the one or more acetylation agents comprise: an N-terminal acyltransferase; and an acyl-CoA. In some embodiments, the N-terminal acyltransferase is N-myristoyltransferase or N-alpha-acetyltransferase. In some embodiments, the acyl-CoA is acetyl-CoA.

In some embodiments, the one or more acylpeptide hydrolases comprise an acylpeptide hydrolase described herein. In some embodiments, the one or more acylpeptide hydrolases comprise an acylpeptide hydrolase comprising an amino acid sequence that is at least 80%, at least 85%, at least 90%, at least 95%, at least 97%, 80-100%, 80-90%, 85-95%, 90-99%, 90-100%, 95-99%, 95-100%, 98-100%, or 100% identical to the amino acid sequence of any one of SEQ ID NOs: 1-25.

In some embodiments, the kit further comprises one or more terminal amino acid recognition molecules (e.g., one or more amino acid binding proteins not having peptide cleavage activity). In some embodiments, the one or more terminal amino acid recognition molecules comprise one or more amino acid binding proteins. In some embodiments, the one or more amino acid binding proteins are selected from ClpS-homologous proteins, UBR-homologous proteins, Ntaq1-homologous proteins, BIR3 domain-homologous proteins, and variants thereof. In some embodiments, each terminal amino acid recognition molecule comprises a detectable label (e.g., a luminescent label, such as a fluorophore dye). In some embodiments, the kit comprises at least two, at least three, at least five, 2-10, 5-10, 5-15, 5-20, or 10-25 terminal amino acid recognition molecules.

In some embodiments, the kit further comprises one or more PTM-specific affinity reagents. In some embodiments, the one or more PTM-specific affinity reagents comprise one or more antibodies that specifically bind to an amino acid comprising a PTM. In some embodiments, each PTM-specific affinity reagent comprises a detectable label (e.g., a luminescent label, such as a fluorophore dye). In some embodiments, the kit comprises at least two, at least three, at least five, 2-10, 5-10, 5-15, 5-20, or 10-25 PTM-specific affinity reagents.

In some embodiments, the kit further comprises instructions for using the kit in a method of polypeptide analysis. In some embodiments, the method of polypeptide analysis is a method described herein.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIGS. 1A-1B provide an example two-step controlled cleavage method of the disclosure. In FIG. 1A, the N-terminal amino acid of a polypeptide is contacted with a modification agent to produce a polypeptide with a modified N-terminal amino acid. In FIG. 1B, the polypeptide with the modified N-terminal amino acid is contacted with an enzyme that catalyzes the removal of the modified N-terminal amino acid to produce a polypeptide having one fewer amino acid (and presenting a new and unmodified amino acid at the N-terminus).

FIGS. 1C-1D illustrate example processes for polypeptide analysis in accordance with the disclosure. FIG. 1C schematically illustrates an example process involving amino acid recognition and controlled cleavage: in panel (1-A), a peptide having an N-terminal tripeptide sequence of R-L-pY is contacted with amino acid recognition molecules, and on-off binding between an R-recognizer and the N-terminus produces a detectable series of signal pulses indicative of arginine; in panel (1-B), a modification agent modifies the N-terminal R; in panel (1-C), an enzyme removes the modified N-terminal R to expose L at the N-terminus; and in panel (2-A), the next cycle of recognition and controlled cleavage begins with terminal amino acid recognition as in panel (1-A). FIG. 1D illustrates an optional pre-recognition process for PTM detection in which, prior to panel (1-A) of FIG. 1C, the peptide is contacted with PTM-specific affinity reagent(s), and on-off binding between a PTM-specific affinity reagent and the peptide produces a detectable series of signal pulses indicative of phospho-tyrosine (pY).

FIGS. 2A-2B provide an example two-step controlled cleavage method of the disclosure. In FIG. 2A, the N-terminal amino acid of a polypeptide is contacted with an acetylation agent (Sulfo-NHS-acetate) to produce a polypeptide with an acetylated N-terminal amino acid. In FIG. 2B, the polypeptide with the acetylated N-terminal amino acid is contacted with an acylpeptide hydrolase (APH) enzyme that catalyzes the removal of the acetylated N-terminal amino acid to produce a polypeptide having one fewer amino acid (and presenting a new and unmodified amino acid at the N-terminus).

FIGS. 3A-3B provide an example two-step controlled cleavage method of the disclosure. In FIG. 3A, the N-terminal amino acid of a polypeptide is contacted with a modification agent (activated handle dipeptide) to produce a polypeptide ligated to the handle dipeptide. In FIG. 3B, the polypeptide ligated to the handle dipeptide is contacted with a tripeptidyl-peptidase enzyme that catalyzes the removal of the three amino acids at the N-terminus of the polypeptide (which includes the handle dipeptide) to produce a polypeptide having one fewer amino acid than the original polypeptide (and presenting a new and unmodified amino acid at the N-terminus).

FIGS. 4A-4C provide graphs showing bulk fluorogenic assays of cleavage activity using Aeropyrum pernix acylpeptide hydrolases (APH2a, APH2b, and APH2c) to selectively function in cleavage of an acetylated leucine substrate (Ac-Leu-AMC) relative to an unmodified leucine substrate (Leu-AMC).

FIG. 5 provides graphs showing the ability of a Bombyx mori acylpeptide hydrolase (APH3a) to selectively function in cleavage of an acetylated alanine substrate (Ac-Ala-AMC) relative to an unmodified alanine substrate (Ala-AMC).

FIG. 6 provides graphs showing experiments using bulk fluorogenic assay using an aminopeptidase (AP) mixture (AP64 and AP37) to selectively function in cleavage of unmodified alanine (Ala-AMC) and leucine substrate (Leu-AMC) relative to acetylated alanine (Ac-Ala-AMC) and leucine substrate (Ac-Leu-AMC).

FIG. 7 provides graphs showing the ability of an acylpeptide hydrolase (APH2) to function in cleavage of an acetylated leucine substrate (Ac-Leu-AMC) at varying concentrations of APH2 and NaCl.

FIG. 8 provides graphs showing the ability of an acylpeptide hydrolase (APH2a) to function in cleavage of an unmodified leucine substrate (Leu-AMC) at varying concentrations of Sulfo-NHS-acetate.

FIG. 9 provides a representative controlled cleavage experiment of a synthetic peptide QP47 (FAAAYPDDDFK (SEQ ID NO: 26)) using an on-chip recognition assay. Provided in the graph is a trace of single-molecule intensity (signal) versus time (frames). Other sequences shown correspond to SEQ ID NOs: 27 (Ac-FAAAYPDDDFK) and 28 (AAAYPDDDFK).

FIG. 10 provides a representative controlled cleavage experiment of a synthetic peptide QP649 (FLAAYPDDDW (SEQ ID NO: 29)) using an on-chip recognition assay. Provided in the graph is a trace of single-molecule intensity (signal) versus time (frames). Other sequences shown correspond to SEQ ID NOs: 30 (Ac-FLAAYPDDDW) and 31 (LAAYPDDDW).

FIG. 11 provides a representative controlled cleavage experiment of a synthetic peptide QP1160 (FLARQAIWAQDDD (SEQ ID NO: 32)) using an on-chip recognition assay. Provided in the graph is a trace of single-molecule intensity (signal) versus time (frames). Other sequences shown correspond to SEQ ID NOs: 33 (Ac-FLARQAIWAQDDD) and 34 (LARQAIWAQDDD).

FIG. 12 provides a representative controlled cleavage experiment of a synthetic peptide QP941 (LRQAFAYPDDD (SEQ ID NO: 35)) using an on-chip recognition assay. Provided in the graph is a trace of single-molecule intensity (signal) versus time (frames). Other sequences shown correspond to SEQ ID NOs: 36 (RQAFAYPDDD) and 37 (QAFAYPDDD).

FIG. 13 provides a representative controlled cleavage experiment of a synthetic peptide QP1160 (FLARQAIWAQDDD (SEQ ID NO: 32)) using an on-chip recognition assay. Provided in the graph is a trace of single-molecule intensity (signal) versus time (frames). Other sequences shown correspond to SEQ ID NOs: 34 (LARQAIWAQDDD) and 38 (ARQAIWAQDDD).

FIGS. 14A-14B provide example results from a cloud-based analysis of an on-chip recognition assay in the absence (FIG. 14A) or presence (FIG. 14B) of APH2a.

FIG. 15 depicts the structure of acylpeptide hydrolase APH2 (based on PDB: 1VE6).

FIGS. 16A-16B provide representative results for APH2d15, APH2b, and APH2d26 in cleavage assays with acetylated alanine substrate (FIG. 16A, top plot), acetylated leucine substrate (FIG. 16A, bottom plot), acetylated phenylalanine substrate (FIG. 16B, top plot), and unmodified phenylalanine substrate (FIG. 16B, bottom plot).

FIGS. 17A-17C provide representative results for APH2b (FIG. 17A), APH2d26 (FIG. 17B), and APH3a (FIG. 17C) in cleavage assays with 19 different acetylated amino acid substrates.

FIGS. 18A-18B provide representative results for on-chip controlled cleavage runs performed with APH2d15 in the left flow cell (FIG. 18A) and APH2d26 in the right flow cell (FIG. 18B). Sequences shown correspond to SEQ ID NOs: 32 (FLARQAIWAQDDD) and 34 (LARQAIWAQDDD).

FIGS. 19A-19B provide representative results for on-chip controlled cleavage runs performed with APH2b in the left flow cell (FIG. 19A) and APH2d26 in the right flow cell (FIG. 19B). Sequences shown correspond to SEQ ID NOs: 32 (FLARQAIWAQDDD) and 34 (LARQAIWAQDDD).

FIGS. 20A-20D provide representative results for cleavage assays carried out with APH2b variants at 50 nM (FIG. 20A), 500 nM (FIG. 20B), and 5 μM (FIG. 20C) with unmodified phenylalanine substrate (Phe) and acetylated phenylalanine substrate (Ac-Phe). FIG. 20D provides a summary ranking of the activity levels for the results shown in FIGS. 20A-20C.

FIGS. 21A-21B provide representative results for APH2b variants (APH2b-G (“G”), APH2b-H (“H”), and APH2b-I (“I”)) in on-chip cutting activity assays.

FIG. 22 depicts a representative automation workflow for peptide sequencing reactions.

FIGS. 23A-23N provide representative analyses of results (FIGS. 23A-23I) and example signal traces (FIGS. 23J-23N) from sequencing runs involving recognition and controlled cleavage of synthetic peptide QP1160 (FLARQAIWAQDDDK* (SEQ ID NO: 39)). SEQ ID NO: 32 (FLARQAIWAQDDD) is also shown.

### DETAILED DESCRIPTION

Aspects of the disclosure relate to methods and associated compositions for controlled cleavage of terminal amino acids from a target polypeptide (e.g., for purposes of polypeptide sequencing). The controlled cleavage methods of the disclosure generally involve at least two distinct steps—(1) modification (or activation) of a terminal amino acid of a polypeptide to produce a modified terminal amino acid for selective removal of that modified terminal amino acid (relative to an unmodified amino acid); and (2) introduction of reaction conditions and/or reagents that result in the removal of the modified terminal amino acid from the polypeptide (without removing a subsequent terminal amino acid that remains unmodified). In the first step, the terminal amino acid can be modified using chemical or enzymatic means in order to produce a polypeptide comprising a modified terminal amino acid (e.g., an acetylated terminal amino acid). In the second step, the modified terminal amino acid can be selectively removed from the polypeptide using an enzyme that catalyzes the removal of the modified terminal amino acid, wherein the enzyme has increased catalytic activity for removal of the modified terminal amino acid relative to a reference polypeptide comprising an unmodified terminal amino acid. These methods of controlled cleavage can be used in combination with polypeptide sequencing methods described herein or known in the art.

For example, the methods of controlled cleavage can be used in combination with peptide sequencing compositions and methods (e.g., dynamic polypeptide sequencing methods) as described in PCT International Publication No. WO 2020/102741 A1, filed Nov. 15, 2019, PCT International Publication No. WO 2021/236983 A2, filed May 20, 2021, PCT International Publication No. WO 2023/122769 A2, filed Dec. 22, 2022, PCT International Publication No. WO 2024/031031 A2, filed Aug. 3, 2023, PCT International Publication No. WO 2024/086832 A1, filed Oct. 20, 2023, PCT International Publication No. WO 2025/101639 A3, filed Nov. 6, 2024, and PCT International Publication No. WO 2025/147658 A1, filed Jan. 3, 2025, each of which is incorporated by reference in its entirety.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

The term “isomers” is intended to include diastereoisomers, enantiomers, regioisomers, structural isomers, rotational isomers, tautomers, and the like. All such isomers of such compounds herein are expressly included in the present invention. When a range of values (“range”) is listed, it encompasses each value and sub-range within the range.

A range is inclusive of the values at the two ends of the range unless otherwise provided. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₄ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified

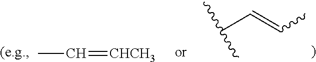

may be in the (E)- or (Z)-configuration.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

The term “halo” or “halogen” refers to fluorine (fluoro, —F), chlorine (chloro, —Cl), bromine (bromo, —Br), or iodine (iodo, —I).

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

As used herein, the term “salt” refers to any and all salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

### Modification Agents

A method of controlled cleavage of a polypeptide (e.g., to facilitate polypeptide sequencing) generally involves a first step of contacting an amino acid (e.g., a terminal amino acid) of a polypeptide with one or more modification agents to produce a polypeptide comprising a modified amino acid (e.g., modified terminal amino acid). A modification agent as used herein is typically a small molecule and/or an enzyme that functions to modify a terminal amino acid of a polypeptide (e.g., modify the backbone (amino or carboxy group) or side chain of a terminal amino acid). In some embodiments, a modification agent is a single small molecule or enzyme. In some embodiments, one or more modification agents consist of 2, 3, 4, or 5 different small molecules and/or enzymes. One or more modification agents comprise or consist of a small molecule and an enzyme that function to modify a terminal amino acid of a polypeptide (e.g., modify the backbone (amino or carboxy group) or side chain of a terminal amino acid) in combination with one another.

A modification agent can modify a terminal amino acid of a polypeptide such that the terminal amino acid comprises a modification selected from the group consisting of: acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination. Accordingly, in some embodiments, the modification agent is an acetylation agent (i.e., an agent that introduces an acetyl group to a terminal amino acid), an ADP-ribosylation agent, a caspase cleavage agent, a citrullination agent, a formylation agent, an N-linked glycosylation agent, an O-linked glycosylation agent, a hydroxylation agent, a methylation agent, a myristoylation agent, a neddylation agent, a nitration agent, an oxidation agent, a palmitoylation agent, a phosphorylation agent, a prenylation agent, a S-nitrosylation agent, a sulfation agent, a sumoylation agent, or a ubiquitination agent.

In some embodiments, an N-terminal amino acid of the polypeptide is contacted with the modification agent, thereby producing a polypeptide comprising a modified N-terminal amino acid. In some embodiments, a C-terminal amino acid of the polypeptide is contacted with the modification agent, thereby producing a polypeptide comprising a modified C-terminal amino acid.

As used herein, in some embodiments, the term modification agent can refer to one molecular component or chemical entity, or a plurality of molecular components or chemical entities, capable of making a desired modification to a terminal amino acid. For example, in some embodiments, a modification agent can refer to a chemical compound capable of reacting with and modifying a terminal amino acid. In some embodiments, a modification agent can refer to an enzyme and a substrate, where the enzyme is capable of catalyzing a reaction involving the substrate and a terminal amino acid that modifies the terminal amino acid.

In some embodiments, a modification agent is an acetylation agent. In some embodiments, the acetylation agent is a succinimidyl acetate compound. Non-limiting examples of succinimidyl acetate compounds include N-Hydroxysulfosuccinimide acetate (e.g., sulfosuccinimidyl acetate (Sulfo-NHS acetate)), N-Hydroxysuccinimide, acetic anhydride, acetyl chloride, and ketene. In some embodiments, an acetylation agent is an N-Hydroxysulfosuccinimide acetate. In some embodiments, an acetylation agent is N-Hydroxysuccinimide acetate. In some embodiments, an acetylation agent is sulfosuccinimidyl acetate (Sulfo-NHS acetate).

In some embodiments, the modification agent is or comprises a compound of Formula (I):

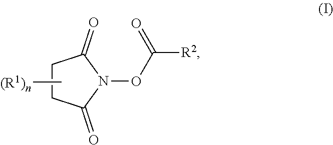

or a salt thereof, wherein:
   each instance of R¹ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂;
   R² is optionally substituted alkyl, optionally substituted carbocyclyl, optionally substituted heterocyclyl, optionally substituted aryl, or optionally substituted heteroaryl;
   each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two instances of R^{A} are joined together with their intervening atom to form an optionally substituted heterocyclic ring or optionally substituted heteroaryl ring; and
n is 0, 1, 2, 3, or 4.

In some embodiments, an acetylation agent is or comprises a compound of Formula (II):

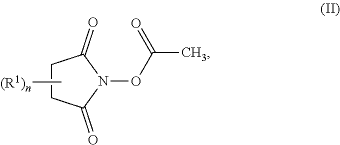

or a salt thereof, wherein:
   each instance of R¹ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂;
   each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two instances of R^{A} are joined together with their intervening atom to form an optionally substituted heterocyclic ring or optionally substituted heteroaryl ring; and
   n is 0, 1, 2, 3, or 4.

In some embodiments, the compound of Formula (I), or salt thereof, is a compound of Formula (II), or salt thereof.

As generally described herein, each instance of R¹ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂.

In some embodiments, at least one instance of R² is —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, or —S(═O)₂N(R^{A})₂. In some embodiments, at least one instance of R² is —S(═O)₂OR^{A}. In some embodiments, at least one instance of R¹ is —S(═O)₂OH.

As generally described herein, R² is optionally substituted alkyl, optionally substituted carbocyclyl, optionally substituted heterocyclyl, optionally substituted aryl, or optionally substituted heteroaryl.

In some embodiments, R² is optionally substituted alkyl. In some embodiments, R² is optionally substituted C₁₋₆ alkyl. In some embodiments, R² is optionally substituted C₁₋₃ alkyl. In some embodiments, R² is unsubstituted C₁₋₆ alkyl. In some embodiments, R² is unsubstituted C₁₋₃ alkyl. In some embodiments, R² is —CH₃.

As generally described herein, each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two instances of R^{A} are joined together with their intervening atom to form an optionally substituted heterocyclic ring or optionally substituted heteroaryl ring.

In some embodiments, at least one instance of R^{A} is hydrogen.

As generally described herein, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0 or 1.

In some embodiments, the compound of Formula (I) is of Formula (I-a) or (I-b):

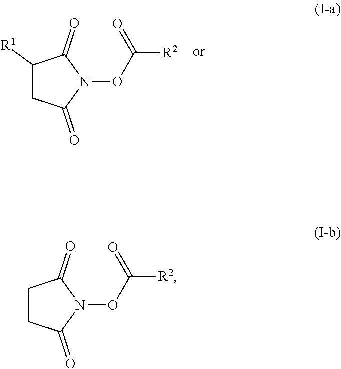

or a salt thereof.

In some embodiments, the compound of Formula (I) or (II) is of Formula (III):

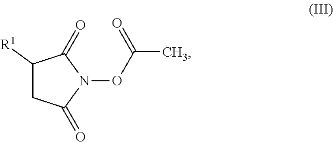

or a salt thereof.

In some embodiments, the compound of Formula (I) or (II) is of formula:

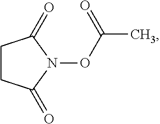 [SMILES: CC(=O)ON1C(=O)CCC1=O]

or a salt thereof.

In some embodiments, the compound of Formula (I), (II), or (III) is of formula:

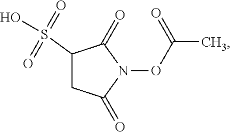 [SMILES: CC(=O)ON1C(=O)CC(S(=O)(=O)O)C1=O]

or a salt thereof.

In some embodiments, the compound of Formula (I), (II), or (III) is of formula:

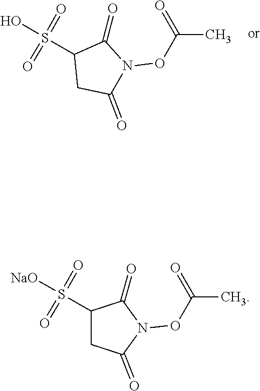

In some embodiments, an acetylation agent is an enzyme that catalyzes the transfer of an acetyl group from a substrate to an amino acid. An enzyme that catalyzes the transfer of an acetyl group from a substrate to an amino acid may be an N-terminal acyltransferase. In some embodiments, the substrate is an acyl-CoA (e.g., acetyl CoA). In some embodiments, the N-terminal acyltransferase is N-myristoyltransferase or N-alpha-acetyltransferase, and the substrate may be an acyl-CoA (e.g., acetyl-CoA).

Contacting a polypeptide with one or more modification agents produces a modified polypeptide (e.g., a polypeptide comprising a modified terminal amino acid). For example, contacting a polypeptide with one or more acetylation agents (e.g., Sulfo-NHS-acetate) produces an acetylated polypeptide (e.g., a polypeptide comprising a acetylated terminal amino acid).

The one or more modification agents may comprise a handle peptide, e.g., a peptide comprising a functional group that permits conjugation of the handle peptide to a polypeptide. The handle peptide may comprise a functional group that permits conjugation to a terminal amino acid of the polypeptide. A handle peptide may comprise or consist of 1, 2, 3, 4, 5, 6, 7, 8, 9, or 10 amino acids. The handle peptide may be an acetylated peptide.

The handle peptide may be ligated to the polypeptide (e.g., the terminal amino acid of the polypeptide) using chemical synthesis methods. For example, a handle peptide can include a Sulfo-NHS acetate such that the Sulfo-NHS acetate permits covalent linkage between the handle peptide and polypeptide.

The handle peptide may be ligated to the polypeptide (e.g., the terminal amino acid of the polypeptide) using enzymatic methods. For example, a handle peptide can be ligated to the terminal amino acid of the polypeptide using subtiligase-catalyzed peptide ligation.

In some embodiments, the one or more modification agents do not comprise an isothiocyanate compound (e.g., phenyl isothiocyanate). In some embodiments, the one or more modification agents do not comprise phenyl isothiocyanate (PITC). For example, in some embodiments, the one or more modification agents do not comprise a compound of the formula: R—N═C═S, where R is substituted or unsubstituted: alkyl (e.g., ethyl), alkenyl, alkynyl, carbocyclyl, heterocyclyl, aryl (e.g., phenyl), heteroaryl, or acyl. In some embodiments, the one or more modification agents do not comprise an enzyme (e.g., an Edmanase enzyme) configured to cleave isothiocyanate-derivatized amino acids (e.g., PITC-derivatized amino acids).

### Enzyme for Removal of Modified Amino Acid

A method of controlled cleavage of a polypeptide (e.g., to facilitate polypeptide sequencing) generally involves a second step of contacting a polypeptide comprising a modified amino acid (e.g., modified terminal amino acid) with an enzyme that catalyzes the removal of the modified amino acid. In some embodiments, an enzyme that catalyzes the removal of the modified amino acid has increased catalytic activity for removal of the modified amino acid relative to a reference polypeptide comprising an unmodified amino acid. An enzyme has increased catalytic activity for the removal of a modified amino acid from a polypeptide relative to a reference polypeptide comprising an unmodified amino acid (e.g., relative to the removal of an unmodified amino acid from a reference polypeptide) if the enzyme demonstrates a catalytic rate of removal of the modified amino acid from the polypeptide that is at least 5%, 10%, 15%, 20%, 25%, 30%, 35%, 40%, or 50% higher than its catalytic rate of removal of the unmodified amino acid of the reference polypeptide. A reference polypeptide may be identical to the polypeptide in every aspect except the modified/unmodified amino acid. In some embodiments, a reference polypeptide comprises an amino acid sequence that is identical to a corresponding amino acid sequence of the polypeptide.

An enzyme that catalyzes the removal of a modified amino acid from a polypeptide may be an enzyme that functions to remove an amino acid that comprises a modification selected from the group consisting of: acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination. In some embodiments, an enzyme that catalyzes the removal of a modified amino acid from a polypeptide is an enzyme that catalyzes the removal of an acetylated amino acid (e.g., an acetylated terminal amino acid).

An enzyme that catalyzes the removal of a modified amino acid from a polypeptide may be a wild-type enzyme or an engineered enzyme. In some embodiments, an enzyme that catalyzes the removal of a modified amino acid is stable and/or functional (i.e., capable of maintaining its three-dimensional structure and/or performing its catalytic function) at elevated temperatures. For example, an enzyme that catalyzes the removal of a modified amino acid may be stable and/or functional at or above 30° C., 35° C., 37° C., 40° C., 45° C., 50° C., 55° C., 60° C., or 65° C. In some embodiments, an enzyme that catalyzes the removal of a modified amino acid is stable and/or functional at temperatures between 4° C. and 80° C., between 10° C. and 70° C., between 20° C. and 70° C., and between 20° C. and 65° C.

In some embodiments, an enzyme that catalyzes the removal of a modified amino acid is capable of removing a modified amino acid from a terminal end of a polypeptide (e.g., N-terminal or C-terminal end of a polypeptide). An enzyme that catalyzes the removal of a modified amino acid is capable of removing a modified alanine, modified arginine, modified asparagine, modified aspartic acid, modified cysteine, modified glutamic acid, modified glutamine, modified glycine, modified histidine, modified isoleucine, modified leucine, modified lysine, modified methionine, modified phenylalanine, modified proline, modified serine, modified threonine, modified tryptophan, modified tyrosine, modified valine, or modified selenocysteine.

Examples of modified amino acids include, without limitation, post-translationally-modified variants (e.g., acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, nitrosylation, sulfation, sumoylation, and ubiquitination), chemically modified variants, unnatural amino acids, and proteinogenic amino acids such as selenocysteine and pyrrolysine. In some embodiments, the modified amino acid is an acetylated alanine, an acetylated arginine, an acetylated asparagine, an acetylated aspartic acid, an acetylated cysteine, an acetylated glutamic acid, an acetylated glutamine, an acetylated glycine, an acetylated histidine, an acetylated isoleucine, an acetylated leucine, an acetylated lysine, an acetylated methionine, an acetylated phenylalanine, an acetylated proline, an acetylated serine, an acetylated threonine, an acetylated tryptophan, an acetylated tyrosine, an acetylated valine, or an acetylated selenocysteine.

In some embodiments, a modified amino acid is a D-amino acid, pyroglutamate, oxidized methionine, cysteine carbamidomethylation, noreleucine, ornithine, aminobutyric acid, 2,3-diaminopropionic acid, citrulline, aminoisobutyric acid, gamma-Carboxyglutamic acid, 2-(4′-Pentenyl)alanine, crotonyl lysine, methyl-lysine, methyl-arginine, homophenylalanine, hydroxy-proline, kynurenine, allyl-glycine, norvaline, diazirine, phosphorylated tyrosine, phosphorylated serine, phosphorylated threonine, nitro-tyrosine, sulfo-tyrosine, L-pyroglutamic acid, or L-3-(1-Naphtyl)-alanine. A D-amino acid can be D-alanine, D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan, D-tyrosine, D-valine, or D-selenocysteine.

An enzyme that catalyzes the removal of an acetylated amino acid (e.g., an acetylated terminal amino acid) may be an acylpeptide hydrolase. Acylpeptide hydrolases (also known as acylamino acid releasing enzyme or acylaminoacyl peptidase [EC3.4.19.1]) are known to catalyze the hydrolysis of an acetylated terminal amino acid from polypeptides (e.g., short acetylated peptides). The acylpeptide hydrolase functions to enzymatically act on a polypeptide to produce an acyl amino acid and a polypeptide with a free terminal end (e.g., free N-terminal end) that is shortened by one amino acid. An acylpeptide hydrolase may be as described in Bartlam et. al., Structure, Vol. 12, 1481-1488, August, 2004; Kiss-Szeman et al., Chem. Sci., 2022, 13, 7132; or Jones, et. al., Proc Natl Acad Sci USA. 1991 Mar. 15; 88(6): 2194-2198.

An acylpeptide hydrolase may be a wild-type acylpeptide hydrolase or an engineered acylpeptide hydrolase. An acylpeptide hydrolase may be a human acylpeptide hydrolase, Sus scrofa acylpeptide hydrolase, Aeropyrum pernix acylpeptide hydrolase, Bombyx mori acylpeptide hydrolase, or Rattus norvegicus acylpeptide hydrolase. In some embodiments, an acylpeptide hydrolase is a wild-type human acylpeptide hydrolase, wild-type Sus scrofa acylpeptide hydrolase, wild-type Aeropyrum pernix acylpeptide hydrolase, wild-type Bombyx mori acylpeptide hydrolase, or wild-type Rattus norvegicus acylpeptide hydrolase. In some embodiments, an acylpeptide hydrolase is an engineered human acylpeptide hydrolase, engineered Sus scrofa acylpeptide hydrolase, engineered Aeropyrum pernix acylpeptide hydrolase, engineered Bombyx mori acylpeptide hydrolase, or engineered Rattus norvegicus acylpeptide hydrolase.

In some embodiments, an engineered acylpeptide hydrolase (e.g., engineered Aeropyrum pernix acylpeptide hydrolase) comprises one or more substitutions, insertions, and/or deletions relative to a wild-type acylpeptide hydrolase. For example, an engineered acylpeptide hydrolase may comprise a deletion of up to 15, 20, or 25 amino acids at the N-terminus of the acylpeptide hydrolase. In some embodiments, an engineered acylpeptide hydrolase comprises a deletion of amino acid residues 1-21 of a wild-type acylpeptide hydrolase (e.g., Aeropyrum pernix acylpeptide hydrolase). In some embodiments, an engineered acylpeptide hydrolase comprises a deletion of at least 5 amino acids between positions 2-60, inclusive, of a wild-type acylpeptide hydrolase (e.g., Aeropyrum pernix acylpeptide hydrolase). For example, an engineered acylpeptide hydrolase may comprise a deletion of positions 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, or 2-60 of a wild-type acylpeptide hydrolase (e.g., Aeropyrum pernix acylpeptide hydrolase).

In some embodiments, an engineered acylpeptide hydrolase (e.g., engineered Aeropyrum pernix acylpeptide hydrolase) comprises one or more amino acid substitutions relative to a wild-type acylpeptide hydrolase. In some embodiments, an engineered acylpeptide hydrolase comprises amino acid substitutions at positions D15 (e.g., D15A substitution) and/or R18 (e.g., R18A substitution) of a wild-type acylpeptide hydrolase (e.g., Aeropyrum pernix acylpeptide hydrolase).

In some embodiments, the acylpeptide hydrolase is an engineered acylpeptide hydrolase (e.g., engineered Aeropyrum pernix acylpeptide hydrolase). In some embodiments, the engineered acylpeptide hydrolase comprises an amino acid sequence that is at least 85% (e.g., at least 85%, at least 90%, at least 95%, at least 96%, at least 97%, at least 98%, at least 99%, or 100%) identical to SEQ ID NO: 1, wherein the amino acid sequence has one or more substitutions, insertions, and/or deletions relative to SEQ ID NO: 1. In some embodiments, the engineered acylpeptide hydrolase comprises a deletion of at least 5 amino acids between positions 2-60, inclusive, relative to SEQ ID NO: 1. In some embodiments, the amino acid sequence comprises a deletion of positions 2-15, 2-21, 2-26, 2-30, or 2-53 relative to SEQ ID NO: 1. In some embodiments, at least a portion of the amino acid sequence is at least 85% identical to positions 61-582 of SEQ ID NO: 1.

In some embodiments, the amino acid sequence further comprises an amino acid substitution at one or more (e.g., 1, 2, 3, 4, 5, or 6) positions corresponding to R113, V471, F485, F488, L492, and T527 of SEQ ID NO: 1. In some embodiments, the amino acid substitution is R113Y or R113N; V471T, V471S, or V471I; F485L or F485M; F488V, F488M, or F488L; L492I; or T527S.

An acylpeptide hydrolase may comprise an amino acid sequence that is at least 80%, at least 85%, at least 90%, at least 95%, at least 97%, 80-90%, 85-95%, 90-100%, 95-100%, 98-100%, or 100% identical to the amino acid sequence of any one of SEQ ID NOs: 1-25.

TABLE 1
Example Acylpeptide Hydrolase (APH) sequences

| | | SEQ |
|---|---|---|
| Name | Amino acid sequence | ID NO |
| Aeropyrum | MRIIMPVEFSRIVRDVERLIAVEKYSLQGVVDGDKLLVVGFSEGSVNAYLYDG | 1 |
| pernix wild- | GETVKLNREPINSVLDPHYGVGRVILVRDVSKGAEQHALFKVNTSRPGEEQRL | |
| type APH | EAVKPMRILSGVDTGEAVVFTGATEDRVALYALDGGGLRELARLPGFGFVSDI | |
| (APH2a) | RGDLIAGLGFFGGGRVSLFTSNLSSGGLRVFDSGEGSFSSASISPGMKVTAGLE | |
| | TAREARLVTVDPRDGSVEDLELPSKDFSSYRPTAITWLGYLPDGRLAVVARRE | |
| | GRSAVFIDGERVEAPQGNHGRVVLWRGKLVTSHTSLSTPPRIVSLPSGEPLLEG | |
| | GLPEDLRRSIAGSRLVWVESFDGSRVPTYVLESGRAPTPGPTVVLVHGGPFAE | |
| | DSDSWDTFAASLAAAGFHVVMPNYRGSTGYGEEWRLKIIGDPCGGELEDVSA | |
| | AARWARESGLASELYIMGYSYGGYMTLCALTMKPGLFKAGVAGASVVDWE | |
| | EMYELSDAAFRNFIEQLTGGSREIMRSRSPINHVDRIKEPLALIHPQNDSRTPLK | |
| | PLLRLMGELLARGKTFEAHIIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 2 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| (APH2b) | DLELPSKDFSSYRPTAITWLGYLPDGRLAVVARREGRSAVFIDGERVEAPQGN | |
| | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAAFRNFIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MRIIMPVEFSRIVRAVEALIAVEKYSLQGVVDGDKLLVVGFSEGSVNAYLYDG | 3 |
| pernix APH | GETVKLNREPINSVLDPHYGVGRVILVRDVSKGAEQHALFKVNTSRPGEEQRL | |
| D15A/R18A | EAVKPMRILSGVDTGEAVVFTGATEDRVALYALDGGGLRELARLPGFGFVSDI | |
| mutant | RGDLIAGLGFFGGGRVSLFTSNLSSGGLRVFDSGEGSFSSASISPGMKVTAGLE | |
| (APH2c) | TAREARLVTVDPRDGSVEDLELPSKDFSSYRPTAITWLGYLPDGRLAVVARRE | |
| | GRSAVFIDGERVEAPQGNHGRVVLWRGKLVTSHTSLSTPPRIVSLPSGEPLLEG | |
| | GLPEDLRRSIAGSRLVWVESFDGSRVPTYVLESGRAPTPGPTVVLVHGGPFAE | |
| | DSDSWDTFAASLAAAGFHVVMPNYRGSTGYGEEWRLKIIGDPCGGELEDVSA | |
| | AARWARESGLASELYIMGYSYGGYMTLCALTMKPGLFKAGVAGASVVDWE | |
| | EMYELSDAAFRNFIEQLTGGSREIMRSRSPINHVDRIKEPLALIHPQNDSRTPLK | |
| | PLLRLMGELLARGKTFEAHIIPDAGHAINTMEDAVKILLPAVFFLATQRERR | |
| Bombyx mori | MSKEFENVIRLYKNFSKVPSIVGAHLSRDTSRVTVKWTVRSTDRGKNTRFVSS | 4 |
| wild-type | YILNESLKVIADCGFGTDISNELLSAVSPNETYRAVIREERNQKDLKKQYLEVW | |
| APH | SKNHLAHCIDLNALDLHGDVYADSEFGCLNWSQDESAIVYVAEKKLAKAEPY | |
| (APH3a) | IVRKPAETPKSDSKETPRKGEEYIYRQDWGEQLVGKHASVIVVCKIESESFTVL | |
| | DGLPEDWCPGQVRFTLDGKSVIGIAWSTEPRRLGLIFCTNRPGHVFTLSLEDDK | |
| | ALSRLSPEGMSVRAARLSPRGAPVWLQRVARGPHHAGHQLVTGNIAEAPLTV | |
| | IVDLVQTVTRTDDGRDFHGIYSQSLPARCFSKDGKRIVFSTPQKNEIRSYVVDIE | |
| | SGGMVDVSIRRAAGSTSVLDVHDDVILAAHSSLTSPSQLYVARLPARGSEAEV | |
| | EWMPVTSQPEVPAGLARSEVTYLQLEHADCADPVKSFSAIYLSPSVQGGSKLP | |
| | LLVWPHGGPHSNFTNSYSFEAAFFNDLGFAILHVNYRGSTGAGEASMAFLPKR | |
| | VGDADVKDCKLATETAVARFGLDESKLCLMGGSHGGFLVAHLSGQYPDLFK | |
| | VVVSRNPVIDVASMENSTDIADWCAVEAGFPFTEEGPPSEEQLLAMRRCSPLV | |
| | HAHKVKAPTALMLGSADKRVPHYQGLEYARRLKANGVATRVYLYDDNHSL | |
| | SSPLVEMDNLINAALWFLKHLDQ | |
| Sus scrofa | MERQVLLSEPEEAAALYRGLSRQPALSAACLGPEVTTQYGGRYRTVHTEWTQ | 5 |
| wild-type | RDLERMENIRFCRQYLVFHDGDSVVFAGPAGNSVETRGELLSRESPSGTMKA | |
| APH (APH1) | VLRKAGGTGTAEEKQFLEVWEKNRKLKSFNLSALEKHGPVYEDDCFGCLSWS | |
| | HSETHLLYVADKKRPKAESFFQTKALDVTGSDDEMARTKKPDQAIKGDQFLF | |
| | YEDWGENMVSKSTPVLCVLDIESGNISVLEGVPESVSPGQAFWAPGDTGVVF | |
| | VGWWHEPFRLGIRFCTNRRSALYYVDLTGGKCELLSDESVAVTSPRLSPDQCR | |
| | IVYLRFPSLVPHQQCGQLCLYDWYTRVTSVVVDIVPRQLGEDFSGIYCSLLPLG | |
| | CWSADSQRVVFDSPQRSRQDLFAVDTQMGSVTSLTAGGSGGSWKLLTIDRDL | |
| | MVVQFSTPSVPPSLKVGFLPPAGKEQAVSWVSLEEAEPFPDISWSIRVLQPPPQ | |
| | QEHVQYAGLDFEAILLQPSNSPEKTQVPMVVMPHGGPHSSFVTAWMLFPAML | |
| | CKMGFAVLLVNYRGSTGFGQDSILSLPGNVGHQDVKDVQFAVEQVLQEEHFD | |
| | AGRVALMGGSHGGFLSCHLIGQYPETYSACVVRNPVINIASMMGSTDIPDWC | |
| | MVEAGFSYSSDCLPDLSVWAAMLDKSPIKYAPQVKTPLLLMLGQEDRRVPFK | |
| | QGMEYYRVLKARNVPVRLLLYPKSTHALSEVEVESDSFMNAVLWLCTHLGS | |
| Rattus | MERQVLLSEPQEAAALYRGLSRQPSLSAACLGPEVTTQYGGLYRTVHTEWTQ | 6 |
| Norvegicus | RDLERMENIRFCRQYLVFHDGDSVVFAGPAGNSVETRGELLSRESPSGTMKA | |
| wild-type | VLRKAGGTVSGEEKQFLEVWEKNRKLKSFNLSALEKHGPVYEDDCFGCLSWS | |
| APH (APH4) | HSETHLLYVAEKKRPKAESFFQTKALDISASDDEMARPKKPDQAIKGDQFVFY | |
| | EDWGETMVSKSIPVLCVLDIDSGNISVLEGVPENVSPGQAFWAPGDTGVVFVG | |
| | WWHEPFRLGIRYCTNRRSALYYVDLSGGKCELLSDGSLAICSPRLSPDQCRIV | |
| | YLQYPCLAPHHQCSQLCLYDWYTKVTSVVVDIVPRQLGESFSGIYCSLLPLGC | |
| | WSADSQRVVFDSAQRSRQDLFAVDTQTGSITSLTAAGSAGSWKLLTIDKDLM | |
| | VAQFSTPSLPPSLKVGFLPPPGKEQSVSWVSLEEAEPIPGIHWGVRVLHPPPDQ | |
| | ENVQYADLDFEAILLQPSNPPDKTQVPMVVMPHGGPHSSFVTAWMLFPAMLC | |
| | KMGFAVLLVNYRGSTGFGQDSILSLPGNVGHQDVKDVQFAVEQVLQEEHFD | |
| | ARRVALMGGSHGGFLSCHLIGQYPETYSACIARNPVINIASMMGSTDIPDWCM | |
| | VETGFPYSNSCLPDLNVWEEMLDKSPIKYIPQVKTPVLLMLGQEDRRVPFKQG | |
| | MEYYRALKARNVPVRLLLYPKSNHALSEVEAESDSFMNAVLWLHTHLGS | |
| Aeropyrum | MRIIMPVEFSRIVRDVERLIAVEKYSLQGVVDGDKLLVVGFSEGSVNAYLYDG | 7 |
| pernix wild- | GETVKLNREPINSVLDPHYGVGRVILVRDVSKGAEQHALFKVNTSRPGEEQRL | |
| type APH | EAVKPMRILSGVDTGEAVVFTGATEDRVALYALDGGGLRELARLPGFGFVSDI | |
| (APH2a) | RGDLIAGLGFFGGGRVSLFTSNLSSGGLRVFDSGEGSFSSASISPGMKVTAGLE | |
| with histidine | TAREARLVTVDPRDGSVEDLELPSKDFSSYRPTAITWLGYLPDGRLAVVARRE | |
| purification | GRSAVFIDGERVEAPQGNHGRVVLWRGKLVTSHTSLSTPPRIVSLPSGEPLLEG | |
| tag | GLPEDLRRSIAGSRLVWVESFDGSRVPTYVLESGRAPTPGPTVVLVHGGPFAE | |
| | DSDSWDTFAASLAAAGFHVVMPNYRGSTGYGEEWRLKIIGDPCGGELEDVSA | |
| | AARWARESGLASELYIMGYSYGGYMTLCALTMKPGLFKAGVAGASVVDWE | |
| | EMYELSDAAFRNFIEQLTGGSREIMRSRSPINHVDRIKEPLALIHPQNDSRTPLK | |
| | PLLRLMGELLARGKTFEAHIIPDAGHAINTMEDAVKILLPAVFFLATQRERRAA | |
| | ALEHHHHHH | |
| Aeropyrum | MVEKYSLQGVVDGDKLLVVGFSEGSVNAYLYDGGETVKLNREPINSVLDPHY | 8 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| (APH2b) | DLELPSKDFSSYRPTAITWLGYLPDGRLAVVARREGRSAVFIDGERVEAPQGN | |
| with histidine | HGRVVLWRGKLVTSHTSLSTPPRIVSLPSGEPLLEGGLPEDLRRSIAGSRLVWV | |
| purification | ESFDGSRVPTYVLESGRAPTPGPTVVLVHGGPFAEDSDSWDTFAASLAAAGFH | |
| tag | VVMPNYRGSTGYGEEWRLKIIGDPCGGELEDVSAAARWARESGLASELYIMG | |
| | YSYGGYMTLCALTMKPGLFKAGVAGASVVDWEEMYELSDAAFRNFIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERRAAALEHHHHHH | |
| Aeropyrum | MRIIMPVEFSRIVRAVEALIAVEKYSLQGVVDGDKLLVVGFSEGSVNAYLYDG | 9 |
| pernix APH | GETVKLNREPINSVLDPHYGVGRVILVRDVSKGAEQHALFKVNTSRPGEEQRL | |
| D15A/R18A | EAVKPMRILSGVDTGEAVVFTGATEDRVALYALDGGGLRELARLPGFGFVSDI | |
| mutant | RGDLIAGLGFFGGGRVSLFTSNLSSGGLRVFDSGEGSFSSASISPGMKVTAGLE | |
| (APH2c) | TAREARLVTVDPRDGSVEDLELPSKDFSSYRPTAITWLGYLPDGRLAVVARRE | |
| with histidine | GRSAVFIDGERVEAPQGNHGRVVLWRGKLVTSHTSLSTPPRIVSLPSGEPLLEG | |
| purification | GLPEDLRRSIAGSRLVWVESFDGSRVPTYVLESGRAPTPGPTVVLVHGGPFAE | |
| tag | DSDSWDTFAASLAAAGFHVVMPNYRGSTGYGEEWRLKIIGDPCGGELEDVSA | |
| | AARWARESGLASELYIMGYSYGGYMTLCALTMKPGLFKAGVAGASVVDWE | |
| | EMYELSDAAFRNFIEQLTGGSREIMRSRSPINHVDRIKEPLALIHPQNDSRTPLK | |
| | PLLRLMGELLARGKTFEAHIIPDAGHAINTMEDAVKILLPAVFFLATQRERRAA | |
| | ALEHHHHHH | |
| Bombyx mori | MSKEFENVIRLYKNFSKVPSIVGAHLSRDTSRVTVKWTVRSTDRGKNTRFVSS | 10 |
| wild-type | YILNESLKVIADCGFGTDISNELLSAVSPNETYRAVIREERNQKDLKKQYLEVW | |
| APH | SKNHLAHCIDLNALDLHGDVYADSEFGCLNWSQDESAIVYVAEKKLAKAEPY | |
| (APH3a) | IVRKPAETPKSDSKETPRKGEEYIYRQDWGEQLVGKHASVIVVCKIESESFTVL | |
| with histidine | DGLPEDWCPGQVRFTLDGKSVIGIAWSTEPRRLGLIFCTNRPGHVFTLSLEDDK | |
| purification | ALSRLSPEGMSVRAARLSPRGAPVWLQRVARGPHHAGHQLVTGNIAEAPLTV | |
| tag | IVDLVQTVTRTDDGRDFHGIYSQSLPARCFSKDGKRIVFSTPQKNEIRSYVVDIE | |
| | SGGMVDVSIRRAAGSTSVLDVHDDVILAAHSSLTSPSQLYVARLPARGSEAEV | |
| | EWMPVTSQPEVPAGLARSEVTYLQLEHADCADPVKSFSAIYLSPSVQGGSKLP | |
| | LLVWPHGGPHSNFTNSYSFEAAFFNDLGFAILHVNYRGSTGAGEASMAFLPKR | |
| | VGDADVKDCKLATETAVARFGLDESKLCLMGGSHGGFLVAHLSGQYPDLFK | |
| | VVVSRNPVIDVASMENSTDIADWCAVEAGFPFTEEGPPSEEQLLAMRRCSPLV | |
| | HAHKVKAPTALMLGSADKRVPHYQGLEYARRLKANGVATRVYLYDDNHSL | |
| | SSPLVEMDNLINAALWFLKHLDQAAALEHHHHHH | |
| Sus scrofa | MERQVLLSEPEEAAALYRGLSRQPALSAACLGPEVTTQYGGRYRTVHTEWTQ | 11 |
| wild-type | RDLERMENIRFCRQYLVFHDGDSVVFAGPAGNSVETRGELLSRESPSGTMKA | |
| APH (APH1) | VLRKAGGTGTAEEKQFLEVWEKNRKLKSFNLSALEKHGPVYEDDCFGCLSWS | |
| with histidine | HSETHLLYVAEKKRPKAESFFQTKALDVTGSDDEMARTKKPDQAIKGDQFLF | |
| purification | YEDWGENMVSKSTPVLCVLDIESGNISVLEGVPESVSPGQAFWAPGDTGVVF | |
| tag | VGWWHEPFRLGIRFCTNRRSALYYVDLTGGKCELLSDESVAVTSPRLSPDQCR | |
| | IVYLRFPSLVPHQQCGQLCLYDWYTRVTSVVVDIVPRQLGEDFSGIYCSLLPLG | |
| | CWSADSQRVVFDSPQRSRQDLFAVDTQMGSVTSLTAGGSGGSWKLLTIDRDL | |
| | MVVQFSTPSVPPSLKVGFLPPAGKEQAVSWVSLEEAEPFPDISWSIRVLQPPPQ | |
| | QEHVQYAGLDFEAILLQPSNSPEKTQVPMVVMPHGGPHSSFVTAWMLFPAML | |
| | CKMGFAVLLVNYRGSTGFGQDSILSLPGNVGHQDVKDVQFAVEQVLQEEHFD | |
| | AGRVALMGGSHGGFLSCHLIGQYPETYSACVVRNPVINIASMMGSTDIPDWC | |
| | MVEAGFSYSSDCLPDLSVWAAMLDKSPIKYAPQVKTPLLLMLGQEDRRVPFK | |
| | QGMEYYRVLKARNVPVRLLLYPKSTHALSEVEVESDSFMNAVLWLCTHLGS | |
| | AAALEHHHHHH | |
| Rattus | MERQVLLSEPQEAAALYRGLSRQPSLSAACLGPEVTTQYGGLYRTVHTEWTQ | 12 |
| Norvegicus | RDLERMENIRFCRQYLVFHDGDSVVFAGPAGNSVETRGELLSRESPSGTMKA | |
| wild-type | VLRKAGGTVSGEEKQFLEVWEKNRKLKSFNLSALEKHGPVYEDDCFGCLSWS | |
| APH (APH4) | HSETHLLYVAEKKRPKAESFFQTKALDISASDDEMARPKKPDQAIKGDQFVFY | |
| with histidine | EDWGETMVSKSIPVLCVLDIDSGNISVLEGVPENVSPGQAFWAPGDTGVVFVG | |
| purification | WWHEPFRLGIRYCTNRRSALYYVDLSGGKCELLSDGSLAICSPRLSPDQCRIV | |
| tag | YLQYPCLAPHHQCSQLCLYDWYTKVTSVVVDIVPRQLGESFSGIYCSLLPLGC | |
| | WSADSQRVVFDSAQRSRQDLFAVDTQTGSITSLTAAGSAGSWKLLTIDKDLM | |
| | VAQFSTPSLPPSLKVGFLPPPGKEQSVSWVSLEEAEPIPGIHWGVRVLHPPPDQ | |
| | ENVQYADLDFEAILLQPSNPPDKTQVPMVVMPHGGPHSSFVTAWMLFPAMLC | |
| | KMGFAVLLVNYRGSTGFGQDSILSLPGNVGHQDVKDVQFAVEQVLQEEHFD | |
| | ARRVALMGGSHGGFLSCHLIGQYPETYSACIARNPVINIASMMGSTDIPDWCM | |
| | VETGFPYSNSCLPDLNVWEEMLDKSPIKYIPQVKTPVLLMLGQEDRRVPFKQG | |
| | MEYYRALKARNVPVRLLLYPKSNHALSEVEAESDSFMNAVLWLHTHLGSAA | |
| | ALEHHHHHH | |
| Aeropyrum | MVERLIAVEKYSLQGVVDGDKLLVVGFSEGSVNAYLYDGGETVKLNREPINS | 13 |
| pernix APH | VLDPHYGVGRVILVRDVSKGAEQHALFKVNTSRPGEEQRLEAVKPMRILSGV | |
| 1-15 amino | DTGEAVVFTGATEDRVALYALDGGGLRELARLPGFGFVSDIRGDLIAGLGFFG | |
| acid deletion | GGRVSLFTSNLSSGGLRVFDSGEGSFSSASISPGMKVTAGLETAREARLVTVDP | |
| (APH2d15) | RDGSVEDLELPSKDFSSYRPTAITWLGYLPDGRLAVVARREGRSAVFIDGERV | |
| | EAPQGNHGRVVLWRGKLVTSHTSLSTPPRIVSLPSGEPLLEGGLPEDLRRSIAG | |
| | SRLVWVESFDGSRVPTYVLESGRAPTPGPTVVLVHGGPFAEDSDSWDTFAASL | |
| | AAAGFHVVMPNYRGSTGYGEEWRLKIIGDPCGGELEDVSAAARWARESGLA | |
| | SELYIMGYSYGGYMTLCALTMKPGLFKAGVAGASVVDWEEMYELSDAAFRN | |
| | FIEQLTGGSREIMRSRSPINHVDRIKEPLALIHPQNDSRTPLKPLLRLMGELLAR | |
| | GKTFEAHIIPDAGHAINTMEDAVKILLPAVFFLATQRERR | |
| Aeropyrum | MLQGVVDGDKLLVVGFSEGSVNAYLYDGGETVKLNREPINSVLDPHYGVGR | 14 |
| pernix APH | VILVRDVSKGAEQHALFKVNTSRPGEEQRLEAVKPMRILSGVDTGEAVVFTG | |
| 1-26 amino | ATEDRVALYALDGGGLRELARLPGFGFVSDIRGDLIAGLGFFGGGRVSLFTSN | |
| acid deletion | LSSGGLRVFDSGEGSFSSASISPGMKVTAGLETAREARLVTVDPRDGSVEDLEL | |
| (APH2d26) | PSKDFSSYRPTAITWLGYLPDGRLAVVARREGRSAVFIDGERVEAPQGNHGRV | |
| | VLWRGKLVTSHTSLSTPPRIVSLPSGEPLLEGGLPEDLRRSIAGSRLVWVESFD | |
| | GSRVPTYVLESGRAPTPGPTVVLVHGGPFAEDSDSWDTFAASLAAAGFHVVM | |
| | PNYRGSTGYGEEWRLKIIGDPCGGELEDVSAAARWARESGLASELYIMGYSY | |
| | GGYMTLCALTMKPGLFKAGVAGASVVDWEEMYELSDAAFRNFIEQLTGGSR | |
| | EIMRSRSPINHVDRIKEPLALIHPQNDSRTPLKPLLRLMGELLARGKTFEAHIIPD | |
| | AGHAINTMEDAVKILLPAVFFLATQRERR | |
| Aeropyrum | MVDGDKLLVVGFSEGSVNAYLYDGGETVKLNREPINSVLDPHYGVGRVILVR | 15 |
| pernix APH | DVSKGAEQHALFKVNTSRPGEEQRLEAVKPMRILSGVDTGEAVVFTGATEDR | |
| 1-30 amino | VALYALDGGGLRELARLPGFGFVSDIRGDLIAGLGFFGGGRVSLFTSNLSSGGL | |
| acid deletion | RVFDSGEGSFSSASISPGMKVTAGLETAREARLVTVDPRDGSVEDLELPSKDFS | |
| (APH2d30) | SYRPTAITWLGYLPDGRLAVVARREGRSAVFIDGERVEAPQGNHGRVVLWRG | |
| | KLVTSHTSLSTPPRIVSLPSGEPLLEGGLPEDLRRSIAGSRLVWVESFDGSRVPT | |
| | YVLESGRAPTPGPTVVLVHGGPFAEDSDSWDTFAASLAAAGFHVVMPNYRGS | |
| | TGYGEEWRLKIIGDPCGGELEDVSAAARWARESGLASELYIMGYSYGGYMTL | |
| | CALTMKPGLFKAGVAGASVVDWEEMYELSDAAFRNFIEQLTGGSREIMRSRS | |
| | PINHVDRIKEPLALIHPQNDSRTPLKPLLRLMGELLARGKTFEAHIIPDAGHAIN | |
| | TMEDAVKILLPAVFFLATQRERR | |
| Aeropyrum | MGETVKLNREPINSVLDPHYGVGRVILVRDVSKGAEQHALFKVNTSRPGEEQ | 16 |
| pernix APH | RLEAVKPMRILSGVDTGEAVVFTGATEDRVALYALDGGGLRELARLPGFGFV | |
| 1-53 amino | SDIRGDLIAGLGFFGGGRVSLFTSNLSSGGLRVFDSGEGSFSSASISPGMKVTAG | |
| acid deletion | LETAREARLVTVDPRDGSVEDLELPSKDFSSYRPTAITWLGYLPDGRLAVVAR | |
| (APH2d53) | REGRSAVFIDGERVEAPQGNHGRVVLWRGKLVTSHTSLSTPPRIVSLPSGEPLL | |
| | EGGLPEDLRRSIAGSRLVWVESFDGSRVPTYVLESGRAPTPGPTVVLVHGGPF | |
| | AEDSDSWDTFAASLAAAGFHVVMPNYRGSTGYGEEWRLKIIGDPCGGELEDV | |
| | SAAARWARESGLASELYIMGYSYGGYMTLCALTMKPGLFKAGVAGASVVD | |
| | WEEMYELSDAAFRNFIEQLTGGSREIMRSRSPINHVDRIKEPLALIHPQNDSRTP | |
| | LKPLLRLMGELLARGKTFEAHIIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 17 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant A | DLELPSKDFSSYRPTAITWLGYLPDGRLAVVARREGRSAVFIDGERVEAPQGN | |
| (APH2b-A) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAALRNFIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 18 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant B | DLELPSKDFSSYRPTAITWLGYLPDGRLAVVARREGRSAVFIDGERVEAPQGN | |
| (APH2b-B) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAALRNVIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 19 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant C | DLELPSKDFSSYRPTAITWLGYLPDGRLAVVARREGRSAVFIDGERVEAPQGN | |
| (APH2b-C) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAALRNMIEQLT | |
| | GGSREIMRSRSPINHVDRIKEPLALIHPQNDSRTPLKPLLRLMGELLARGKTFEA | |
| | HIIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 20 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant D | DLELPSKDFSSYRPTAITWLGYLPDGRLAVVARREGRSAVFIDGERVEAPQGN | |
| (APH2b-D) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAALRNFIEQITG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 21 |
| pernix APH | GVGRVILVRDVSKGAEQHALFKVNTSRPGEEQRLEAVKPMYILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant E | DLELPSKDFSSYRPTAITWLGYLPDGRLAVVARREGRSAVFIDGERVEAPQGN | |
| (APH2b-E) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAALRNLIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 22 |
| pernix APH | GVGRVILVRDVSKGAEQHALFKVNTSRPGEEQRLEAVKPMNILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant F | DLELPSKDFSSYRPTAITWLGYLPDGRLAVVARREGRSAVFIDGERVEAPQGN | |
| (APH2b-F) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASVVDWEEMYELSDAAMRNLIEQLT | |
| | GGSREIMRSRSPINHVDRIKEPLALIHPQNDSRTPLKPLLRLMGELLARGKTFEA | |
| | HIIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 23 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant G | DLELPSKDFSSYRPTAITWLGYLPDGRLAVVARREGRSAVFIDGERVEAPQGN | |
| (APH2b-G) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASTVDWEEMYELSDAAFRNFIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 24 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant H | DLELPSKDFSSYRPTAITWLGYLPDGRLAVVARREGRSAVFIDGERVEAPQGN | |
| (APH2b-H) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASSVDWEEMYELSDAAFRNFIEQLTG | |
| | GSREIMRSRSPINHVDRIKEPLALIHPQNDSRTPLKPLLRLMGELLARGKTFEAH | |
| | IIPDAGHAINTMEDAVKILLPAVFFLATQRERR | |
| Aeropyrum | MVEKYSLQGVVDGDKLLVVGFSEGSVNAYLYDGGETVKLNREPINSVLDPHY | 25 |
| pernix APH | GVGRVILVRDVSKGAEQHALFKVNTSRPGEEQRLEAVKPMRILSGVDTGEAV | |
| 1-21 amino | VFTGATEDRVALYALDGGGLRELARLPGFGFVSDIRGDLIAGLGFFGGGRVSL | |
| acid deletion | FTSNLSSGGLRVFDSGEGSFSSASISPGMKVTAGLETAREARLVTVDPRDGSVE | |
| variant I | DLELPSKDFSSYRPTAITWLGYLPDGRLAVVARREGRSAVFIDGERVEAPQGN | |
| (APH2b-I) | HGRVVLWRGKLVTSHTSLSTPPRIVSLPSGEPLLEGGLPEDLRRSIAGSRLVWV | |
| | ESFDGSRVPTYVLESGRAPTPGPTVVLVHGGPFAEDSDSWDTFAASLAAAGFH | |
| | VVMPNYRGSTGYGEEWRLKIIGDPCGGELEDVSAAARWARESGLASELYIMG | |
| | YSYGGYMTLCALTMKPGLFKAGVAGASIVDWEEMYELSDAALRNLIEQLTG | |
| | GSREIMRSRSPINHVDRIKEPLALIHPQNDSRSPLKPLLRLMGELLARGKTFEAH | |
| | IIPDAGHAINTMEDAVKILLPAVFFLATQRERR | |

As described herein, in some embodiments, an acylpeptide hydrolase of the disclosure comprises an amino acid sequence that shares a percentage of sequence identity with an amino acid sequence selected from Table 1. For the purposes of comparing two or more amino acid sequences, the percentage of “sequence identity” between a first amino acid sequence and a second amino acid sequence (also referred to herein as “amino acid identity”) may be calculated by: dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position).

Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more amino acid sequences, refer to two or more sequences or subsequences that are the same. Two sequences are “substantially identical” if two sequences have a specified percentage of amino acid residues that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or “percent alignment” in the context of two or more amino acid sequences, refer to two or more sequences or subsequences that are the same. Two sequences are “substantially aligned” if two sequences have a specified percentage of amino acid residues that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

An acylpeptide hydrolase may be stable and/or functional at or above 30° C., 35° C., 37° C., 40° C., 45° C., 50° C., 55° C., 60° C., or 65° C. In some embodiments, an acylpeptide hydrolase is stable and/or functional at temperatures between 4° C. and 80° C., between 10° C. and 70° C., between 20° C. and 70° C., and between 20° C. and 65° C. In some embodiments, an acylpeptide hydrolase is capable of removing an acetylated serine, acetylated methionine, acetylated leucine, or acetylated alanine from a polypeptide.

In some embodiments, an enzyme that catalyzes the removal of a modified amino acid (e.g., a modified terminal amino acid) is a peptidase. For example, in embodiments of the method that utilize a handle peptide (e.g., methods that involve ligation of a handle peptide to a terminal end of a polypeptide), the method may utilize a peptidase to remove the length of the handle peptide and the terminal amino acid of the polypeptide. Thus, in some embodiments, the method involves ligation of a handle peptide consisting of a single amino acid to the terminal end of a polypeptide in a first step followed by contacting the modified polypeptide with a dipeptidyl-peptidase (to remove two amino acids, including the handle peptide, from the polypeptide). In some embodiments, the method involves ligation of a handle peptide consisting of two amino acid to the terminal end of a polypeptide in a first step followed by contacting the modified polypeptide with a tripeptidyl-peptidase (to remove three amino acids, including the handle peptide, from the polypeptide). In some embodiments, a handle peptide comprises a modification (e.g., an acetyl group or acetylation). In some embodiments, a polypeptide that is ligated to a handle peptide comprising an acetylation is contacted with a deacetylase prior to contacting with a peptidase.

In some embodiments, an enzyme that catalyzes the removal of a modified amino acid (e.g., a modified terminal amino acid) is not an enzyme configured to cleave isothiocyanate-derivatized amino acids (e.g., PITC-derivatized amino acids). In some embodiments, an enzyme that catalyzes the removal of a modified amino acid (e.g., a modified terminal amino acid) is not a serine protease (e.g., an Edmanase).

### Polypeptides and Terminal Amino Acids

A polypeptide (also referred to as a “protein” or “peptide”) for use in a method described herein comprises a polymer of amino acid residues linked together by peptide bonds. The terms refer to proteins, polypeptides, and peptides of any size, structure, or function. Typically, a protein or peptide will be at least ten amino acids in length. In some embodiments, a polypeptide is between about 5 and about 1000 amino acids in length (e.g., between about 50 and about 250, between about 100 and about 500, between about 150 and about 700, or between about 200 and about 400 amino acids in length). In some embodiments, a polypeptide is between about 100 and about 2000 amino acids in length (e.g., between about 10 and about 300, between about 100 and about 400, between about 100 and about 500, or between about 500 and about 2000 amino acids in length). In some embodiments, a polypeptide is between about 5 and about 100 amino acids in length (e.g., between about 5 and about 80, between about 5 and about 50, between about 10 and about 100, between about 10 and about 50, or between about 15 and about 60 amino acids in length).

In some embodiments, a plurality of polypeptides can refer to a plurality of polypeptide molecules, where each polypeptide molecule of the plurality comprises an amino acid sequence that is different from any other polypeptide molecule of the plurality. In some embodiments, a plurality of polypeptides can include at least 1 peptide and up to 1,000 peptides (e.g., at least 1 polypeptide and up to 10, 50, 100, 250, or 500 polypeptides). In some embodiments, a plurality of polypeptides comprises 1-5, 5-10, 1-15, 15-20, 10-100, 50-250, 100-500, 500-1,000, or more, different polypeptides. A protein may refer to an individual protein or a collection of proteins. Certain proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein or peptide may be a fragment of a naturally occurring protein or peptide. A protein may be naturally occurring, recombinant, synthetic, or any combination of these.

A polypeptide has two terminal amino acids, an N-terminal amino acid and a C-terminal amino acid. In some embodiments of the disclosure, a polypeptide is modified with a modification agent at the N-terminal amino acid of the polypeptide and/or the C-terminal amino acid of the polypeptide is immobilized to a surface. In other embodiments of the disclosure, a polypeptide is modified with a modification agent at the C-terminal amino acid of the polypeptide and/or the N-terminal amino acid of the polypeptide is immobilized to a surface. Thus, in certain embodiments, a polypeptide is immobilized to the surface through a terminal amino acid, wherein (i) the N-terminal amino acid of the polypeptide is contacted with the modification agent and the C-terminal amino acid is immobilized to the surface. Alternatively, a polypeptide may be immobilized to the surface through a terminal amino acid, wherein the C-terminal amino acid of the polypeptide is contacted with the modification agent and the N-terminal amino acid is immobilized to the surface. In some embodiments, a polypeptide is immobilized to the surface through the carboxyl group of the C-terminal amino acid. In some embodiments, a polypeptide is immobilized to the surface through the amino group of the N-terminal amino acid. In some embodiments, a polypeptide is immobilized to the surface through a side chain of an amino acid (e.g., lysine) at a terminal or internal position of the polypeptide.

Immobilizing a polypeptide on a surface (e.g., a surface of a substrate, e.g., of a solid support such as an array or a chip). In some embodiments, a polypeptide may be immobilized on a surface of a sample well (e.g., on a bottom surface of a sample well) on a substrate. In some embodiments, the N-terminal amino acid of the polypeptide is immobilized (e.g., attached to the surface). In some embodiments, the C-terminal amino acid of the polypeptide is immobilized (e.g., attached to the surface). In some embodiments, one or more non-terminal amino acids are immobilized (e.g., attached to the surface). The immobilized amino acid(s) can be attached using any suitable covalent or non-covalent linkage, e.g., as described in this application. In some embodiments, a plurality of polypeptides are attached to a plurality of sample wells (e.g., with one polypeptide attached to a surface, for example a bottom surface, of each sample well), for example in an array of sample wells on a substrate.

The polypeptide may be immobilized to a surface through a linker. The linker may comprise one or more biomolecules. In some embodiments, a linker is an oligonucleotide (e.g., an oligonucleotide consisting of at least 5, at least 10, at least 15, 1-20, 5-50, or 10-30 nucleotides). In some embodiments, the oligonucleotide comprises a first oligonucleotide strand hybridized to a second oligonucleotide strand. In other embodiments, a linker is a peptide linker (e.g., a peptide linker consisting of 1-15 amino acids). A peptide linker may comprise a series of glycine and/or serine residues. In some embodiments, a linker comprises a protease cleavage site, which may contain 2-5 amino acid residues that are recognizable and/or cleavable by a suitable protease.

In some examples, the linker may comprise a functional group that can form a covalent bond with the polypeptide. Exemplary functional groups include, but are not limited to, a maleimide group, an iodoacetamide group, a vinyl sulfone group, an acrylate group, an acrylamide group, an acrylonitrile group, or a methacrylate group. In some instances, the linker can contain one or more reactive amines including, but are not limited to, acetyl-lysine-valine-citrulline-p-aminobenzyloxycarbonyl (AcLys-VC-PABC) or amino PEG6-propionyl. Other exemplary linkers include Sulfosuccinimidyl-4-[Nmaleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus).

In some embodiments, a polypeptide is immobilized to a surface via an intermediate linker that links (e.g., covalently or non-covalently) the polypeptide to the surface. The intermediate linker may comprise biotin and/or streptavidin. For example, in some embodiments, a polypeptide and a surface may each be biotinylated (e.g., linked to at least one biotin molecule) and linked to each other through biotin binding to an intermediate streptavidin molecule. In some embodiments, a polypeptide is biotinylated and the surface is attached to a streptavidin molecule.

In some embodiments, a polypeptide is immobilized to a surface through a linker comprising an oligonucleotide, an avidin protein, and one or more biotin moieties. For example, in some embodiments, the polypeptide is conjugated to a single- or double-stranded oligonucleotide comprising a biotin moiety, and the biotin moiety is bound by an avidin protein (e.g., streptavidin) attached to the surface. In some embodiments, the polypeptide comprises a first biotin moiety, the surface is a biotinylated surface comprising a second biotin moiety, and the first and second biotin moieties are bound to different biotin binding sites on an avidin protein (e.g., forming a non-covalent linkage between the polypeptide and the surface). In some embodiments, the polypeptide is conjugated to a single- or double-stranded oligonucleotide that comprises the first biotin moiety.

In certain single molecule methods, a polypeptide to be subjected to a method of the disclosure is immobilized to a surface such that the polypeptide may be monitored without interference from other reaction components in solution. In some embodiments, surface immobilization of the polypeptide allows the polypeptide to be confined to a desired region of a surface for real-time monitoring of a reaction involving the polypeptide. In some embodiments, a solid support comprises a plurality of sample wells comprising a surface. In some embodiments, the methods comprise immobilizing a single polypeptide to a surface of each of a plurality of sample wells. In some embodiments, confining a single polypeptide per sample well is advantageous for single molecule detection methods, e.g., single molecule polypeptide sequencing.

As used herein, in some embodiments, a surface refers to a surface of a substrate or solid support. In some embodiments, a solid support refers to a material, layer, or other structure having a surface, such as a receiving surface, that is capable of supporting a deposited material, such as a functionalized peptide described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one-dimensional array is an array having one column or row of elements in the first dimension and a plurality of columns or rows in the second dimension. The number of columns or rows in the first and second dimensions may or may not be the same.

In some embodiments, the sample well is formed by a bottom surface comprising a non-metallic layer and side wall surfaces comprising a metallic layer. In some embodiments, the non-metallic layer comprises a transparent layer (e.g., glass, silica). In some embodiments, the metallic layer comprises a metal oxide surface (e.g., titanium dioxide). In some embodiments, the metallic layer comprises a passivation coating (e.g., a phosphorus-containing layer, such as an organophosphonate layer). In some embodiments, the bottom surface comprising the non-metallic layer comprises a functional moiety that is complementary to a functional moiety of a polypeptide for immobilization to the bottom surface. Methods of selective surface modification and functionalization are described in further detail in U.S. Patent Publication No. 2018/0326412 and U.S. Patent Publication No. 2021/0129179 (based on U.S. Provisional Application No. 62/914,356), the contents of each of which are hereby incorporated by reference.

In some embodiments, a polypeptide comprising a functional moiety (e.g., a functionalized side chain or terminal end) is contacted with a complementary functional moiety of the solid support to form a covalent or non-covalent linkage group. In some embodiments, the functional moiety (e.g., functionalized side chain or terminal end) and the complementary functional moiety comprise partner click chemistry handles, e.g., which form a covalent linkage group between the polypeptide and the solid support. Suitable click chemistry handles are described elsewhere herein and known in the art. See, e.g., Becer, Hoogenboom, and Schubert, Click Chemistry beyond Metal-Catalyzed Cycloaddition, Angewandte Chemie International Edition (2009) 48: 4900-4908 and PCT/US2012/044584 and references therein, which references are incorporated herein by reference for click chemistry handles and methodology. In some embodiments, the functional moiety (e.g., functionalized side chain or terminal end) and the complementary functional moiety comprise non-covalent binding partners, e.g., which form a non-covalent linkage group between the polypeptide and the solid support. Examples of non-covalent binding partners include complementary oligonucleotide strands (e.g., complementary nucleic acid strands, including DNA, RNA, and variants thereof), protein-protein binding partners (e.g., barnase and barstar), and protein-ligand binding partners (e.g., biotin and streptavidin).

In some embodiments, in a polypeptide sequencing method of the disclosure, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

### Compositions and Reaction Mixtures

In some aspects, the disclosure provides compositions and kits comprising at least one cleaving reagent (e.g., one or more hydrolases and/or peptidases) described herein. In some embodiments, a composition comprises two or more cleaving reagents, where at least one cleaving reagent comprises a peptidase (e.g., an aminopeptidase, a dipeptidyl-peptidase, or a tripeptidyl-peptidase) and, optionally a tag sequence, described herein. In some embodiments, a composition comprises two or more cleaving reagents, where at least one cleaving reagent comprises an acylpeptide hydrolase described herein. In some embodiments, a composition comprises two or more cleaving reagents described herein.

In some embodiments, the first, second, and third cleaving reagents are present in the composition at a first, second, and third concentration, respectively, where the first concentration is at least two-fold higher than the second concentration. In some embodiments, the first concentration is at least five-fold higher than the third concentration. In some embodiments, the second concentration is at least two-fold higher than the third concentration.

In some embodiments, the molar ratio of the first cleaving reagent to the second cleaving reagent in the composition is between about 10:1 and about 500:1 (e.g., between about 50:1 and about 500:1, between about 100:1 and about 500:1, between about 200:1 and about 400:1, between about 250:1 and about 350:1, or between about 275:1 and about 325:1). In some embodiments, the molar ratio of the first cleaving reagent to the second cleaving reagent in the composition is about 300:1. In some embodiments, the molar ratio of the first cleaving reagent to the second cleaving reagent in the composition is between about 2:1 and about 20:1 (e.g., between about 2:1 and about 15:1, between about 2:1 and about 10:1, between about 4:1 and about 15:1, or between about 5:1 and about 10:1). In some embodiments, the molar ratio of the first cleaving reagent to the third cleaving reagent in the composition is between about 5:1 and about 200:1 (e.g., between about 5:1 and about 150:1, between about 5:1 and about 100:1, between about 10:1 and about 80:1, or between about 10:1 and about 50:1).

In some embodiments, the first cleaving reagent is present at a concentration of between about 10 μM and about 100 μM (e.g., 20-80 μM, 20-60 μM, 20-40 μM, 10-30 μM, 25-35 μM, 30-50 μM, 50-70 μM, 70-90 μM). In some embodiments, the first cleaving reagent is present at a concentration of about 20 μM, about 30 μM, about 40 μM, about 60 PM, or about 80 PM. In some embodiments, the first cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a charged side chain (e.g., arginine, lysine, aspartate, glutamate).

In some embodiments, the second cleaving reagent is present at a concentration of between about 0.01 μM and about 10 μM (e.g., between about 0.01 μM and about 5 μM, between about 0.01 μM and about 1 μM, or between about 0.05 μM and about 0.5 μM). In some embodiments, the second cleaving reagent is present at a concentration of between about 0.1 μM and about 25 μM (e.g., 0.1-10 μM, 0.5-20 μM, 1-10 μM, 2-20 μM, 2-15 μM, 1-8 μM). In some embodiments, the second cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a hydrophobic side chain (e.g., alanine, valine, isoleucine, leucine, methionine, phenylalanine, tyrosine, tryptophan).

In some embodiments, the third cleaving reagent is present at a concentration of between about 0.01 μM and about 25 μM (e.g., 0.1-10 μM, 0.5-20 μM, 1-10 μM, 2-20 μM, 2-15 μM, 1-8 μM). In some embodiments, the third cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the polypeptide comprises an XP dipeptide motif, where: X is the N-terminal amino acid, and P is a proline amino acid.

In some aspects, the disclosure provides a reaction mixture for polypeptide analysis, the reaction mixture comprising: a composition described herein; and one or more amino acid recognizers (e.g., one or more amino acid binding proteins not having peptide cleavage activity). In some embodiments, an amino acid recognizer comprises an amino acid binding protein, such as a ClpS protein (e.g., Planctomycetia bacterium ClpS protein), a UBR protein (e.g., Kluyveromyces marxianus UBR protein), an Ntaq1 protein (e.g., Scleropages formosus Ntaq1 protein), or a variant or homolog thereof. In some embodiments, an amino acid recognizer comprises a label (e.g., a detectable label, such as a luminescent label). Examples of amino acid recognizers (e.g., recognition molecules) are described in detail in PCT International Publication No. WO2020/102741A1, filed Nov. 15, 2019, PCT International Publication No. WO2021/236983A2, filed May 20, 2021, PCT International Publication No. WO 2023/122769 A2, filed Dec. 22, 2022, PCT International Publication No. WO 2024/031031 A2, filed Aug. 3, 2023 (based on priority application U.S. Ser. No. 63/395,328, filed Aug. 4, 2022), PCT International Publication No. WO 2024/086832 A1, filed Oct. 20, 2023, PCT International Publication No. WO 2025/101639 A3, filed Nov. 6, 2024, and PCT International Publication No. WO 2025/147658 A1, filed Jan. 3, 2025, the relevant content of each of which is incorporated by reference in its entirety.

As described herein, compositions and reaction mixtures of the disclosure can be used to determine at least one chemical characteristic of a polypeptide based on a characteristic pattern. In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognizer to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., aminopeptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

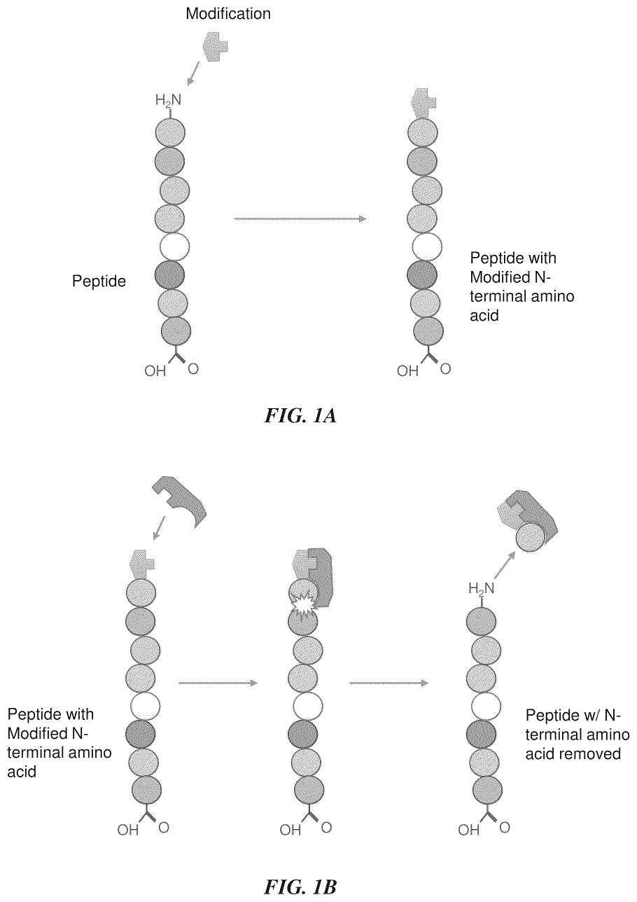

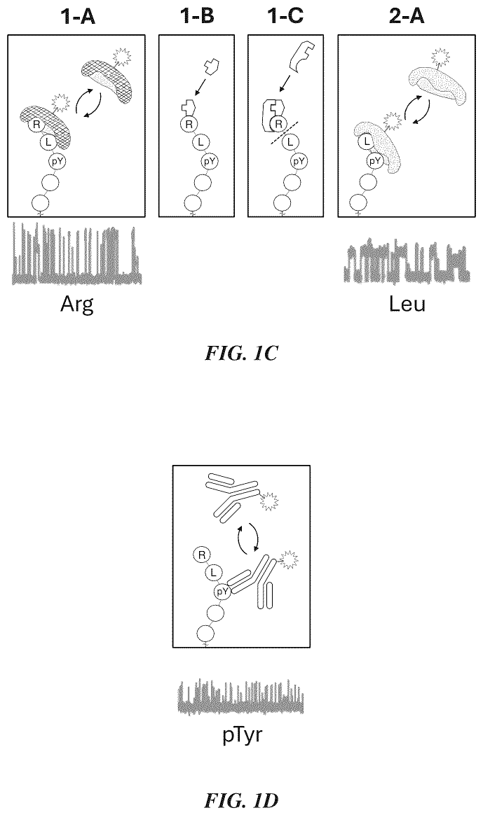

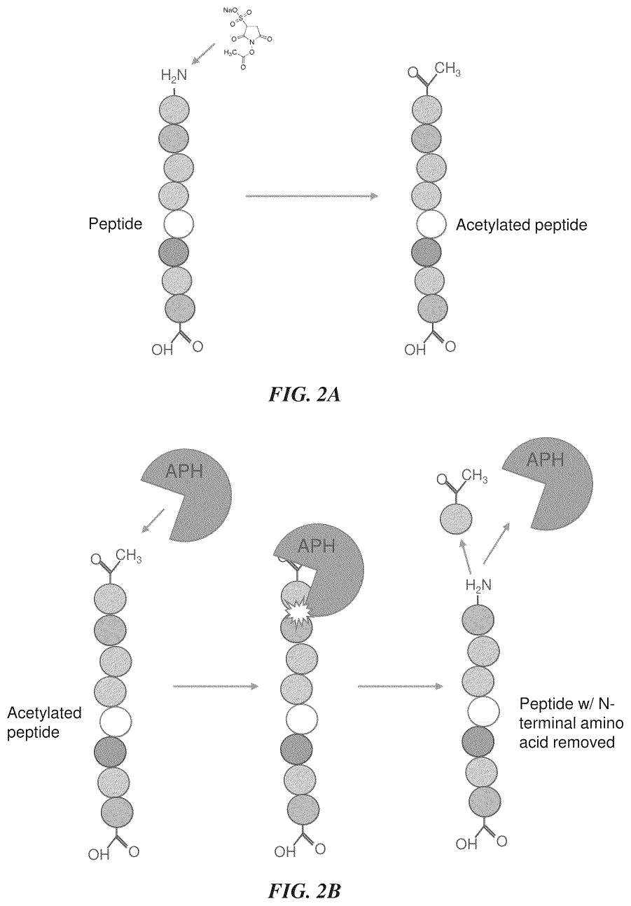

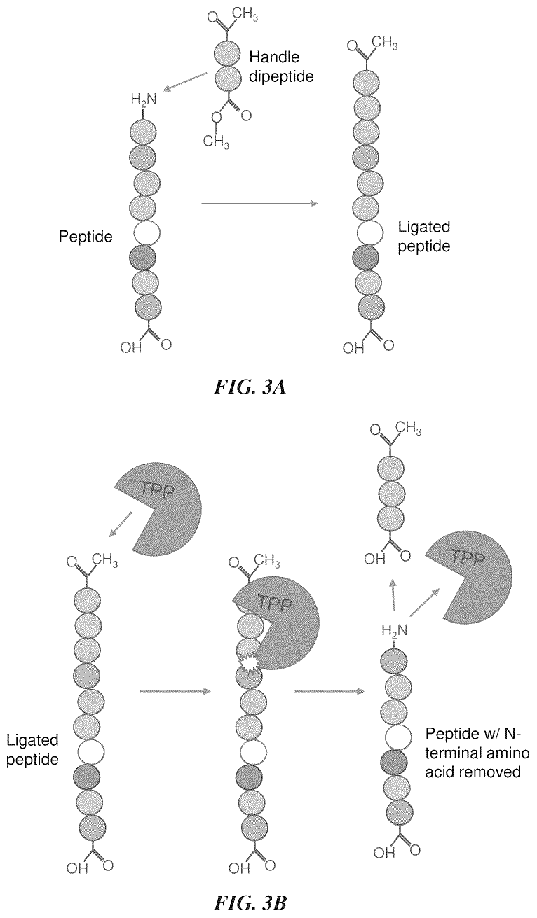

In some aspects, the methods of the disclosure comprise one or more cycles of amino acid recognition and amino acid cleavage. FIG. 1C schematically illustrates an example process of recognition and controlled cleavage in accordance with the methods described herein. Panels (1-A)-(1-C) illustrate a first cycle of recognition and controlled cleavage.

As shown in panel (1-A), in some embodiments, a first step of recognition comprises contacting a polypeptide with one or more amino acid recognition molecules, and monitoring a signal for a series of signal pulses corresponding to binding interactions between the amino acid recognition molecule(s) and a terminus of the polypeptide. In some embodiments, the series of signal pulses is indicative of at least one chemical characteristic of the polypeptide, such as the identity of the amino acid at the terminus of the polypeptide (e.g., the terminal amino acid).

In some embodiments, recognition is performed for at least 30 seconds, at least 1 minute, at least 2 minutes, at least 5 minutes, 1-30 minutes, 2-20 minutes, 5-30 minutes, 5-15 minutes, or about 10 minutes. In some embodiments, recognition is performed at any temperature suitable for amino acid recognition molecule binding and the detection thereof. In some embodiments, recognition is performed at a temperature of at least 10° C., at least 15° C., at least 20° C., at least 25° C., or at least 30° C. In some embodiments, recognition is performed at a temperature of between about 10° C. and about 50° C. (e.g., 15-45° C., 20-40° C., at or around 25° C., at or around 30° C., at or around 35° C., at or around 37° C.). In some embodiments, recognition is performed at a temperature of about 25° C.

In some embodiments, a wash step is performed following recognition and prior to controlled cleavage. In some embodiments, the wash step is performed to remove amino acid recognition molecules. Accordingly, in some embodiments, the wash step is performed using any suitable solution that does not include the amino acid recognition molecules (e.g., a solution comprising water, a buffer, and/or a salt).

As shown in panels (1-B)-(1-C), in some embodiments, a step of controlled cleavage comprises modifying the terminal amino acid of the polypeptide (1-B), and removing the modified terminal amino acid from the polypeptide (1-C). In some embodiments, a wash step is performed between steps of modifying and removing. In some embodiments, the wash step is performed to remove excess modification agent(s). Accordingly, in some embodiments, the wash step is performed using any suitable solution that does not include the one or more modification agents (e.g., a solution comprising water, a buffer, and/or a salt).

In some embodiments, the terminal amino acid is modified by contacting the polypeptide with one or more modification agents described herein, and the modified terminal amino acid is removed by contacting the polypeptide with one or more enzymes described herein. For example, in some embodiments, the terminal amino acid is acetylated using one or more acetylation agents, and the acetylated terminal amino acid is cleaved using an acylpeptide hydrolase.

In some embodiments, acetylation is performed using one or more succinimidyl acetate compounds described herein, such as a compound of any one of Formulae (I), (I-a), (I-b), (II), and (III). In some embodiments, acetylation is performed by exposing the polypeptide to a solution containing a succinimidyl acetate compound at a concentration of between about 1 mM and about 50 mM (e.g., 5-40 mM, 10-30 mM, 15-25 mM, or about 20 mM) for at least 5 minutes (e.g., 5-40, 10-30, 15-25, or about 20 minutes). In some embodiments, acetylation is performed at a temperature of at least 25° C., at least 30° C., at least 35° C., at least 40° C., or at least 45° C. In some embodiments, acetylation is performed at a temperature of between about 35° C. and about 55° C. (e.g., 35-50° C., 40-50° C., at or around 40° C., at or around 45° C., at or around 50° C.). In some embodiments, acetylation is performed at a temperature of about 45° C.

In some embodiments, cleavage of acetylated terminal amino acids is performed using one or more acylpeptide hydrolases, such as an acylpeptide hydrolase identical or homologous to any one of SEQ ID NOs: 1-25. In some embodiments, cleavage is performed by exposing the polypeptide to a solution containing an acylpeptide hydrolase at a concentration of between about 100 μM and about 1,000 μM (e.g., 100-800 μM, 300-800 μM, 200-600 μM, 250-400 PM, 300-500 μM, or about 400 μM) for at least 20 minutes (e.g., 20-60, 30-50, 40-50, or about 45 minutes). In some embodiments, cleavage is performed at a temperature of at least 25° C., at least 30° C., at least 35° C., at least 40° C., or at least 45° C. In some embodiments, cleavage is performed at a temperature of between about 35° C. and about 55° C. (e.g., 35-50° C., 40-50° C., at or around 40° C., at or around 45° C., at or around 50° C.). In some embodiments, cleavage is performed at a temperature of about 45° C. In some embodiments, acetylation and cleavage is performed at or about the same temperature.

In some embodiments, a wash step is performed following controlled cleavage and prior to recognition. In some embodiments, the wash step is performed to remove modification agent(s) and/or enzyme(s). Accordingly, in some embodiments, the wash step is performed using any suitable solution that does not include the modification agent(s) and/or enzyme(s) (e.g., a solution comprising water, a buffer, and/or a salt). Panel (2-A) illustrates a second step of recognition to begin the next cycle of recognition and controlled cleavage, which may be performed as described for the first cycle.

In some embodiments, polypeptides may be further analyzed prior to the one or more cycles of recognition and controlled cleavage described herein. For example, FIG. 1D illustrates an example process of evaluating a polypeptide for the presence of one or more post-translational modifications (PTMs). As shown relative to the example process of FIG. 1C, in some embodiments, the optional PTM detection (e.g., PTM recognition) can be performed prior to the first step of recognition (1-A). In some embodiments, PTM recognition comprises contacting a polypeptide with one or more PTM-specific affinity reagents, and monitoring a signal for a series of signal pulses corresponding to binding interactions between the PTM-specific affinity reagent(s) and the polypeptide. In some embodiments, the series of signal pulses is indicative of the presence of at least one PTM in the polypeptide. In some embodiments, such methods can be used to characterize different proteoforms of a polypeptide in a sample comprising a plurality of polypeptides. Examples of PTM-specific affinity reagents and methods of PTM detection prior to sequencing are described in detail in PCT International Publication No. WO 2025/101639, filed Nov. 6, 2024, the contents of which are incorporated by reference in their entirety.

Post-translational modifications (PTMs) are modifications that occur on a protein, typically catalyzed by enzymes, after translation of the protein. A PTM generally refers to the covalent addition of a functional group to a protein, proteolytic cleavage of a protein, or degradation of one or more regions of a protein. Examples of PTMs are known in the art and include, without limitation, phosphorylation, glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, and ubiquitination.

In some embodiments, PTM recognition comprises contacting a polypeptide with one or more PTM-specific affinity reagents. A PTM-specific affinity reagent is a molecule that binds to an amino acid comprising a post-translational modification (PTM). In some embodiments, the PTM-specific affinity reagent specifically binds to an amino acid comprising a PTM (e.g., binds to the amino acid having a PTM with a higher affinity than the same amino acid without the PTM). PTM-specific affinity reagents include, for example, proteins and nucleic acids, which may be synthetic or recombinant. In some embodiments, a PTM-specific affinity reagent is an antibody (e.g., a single-chain antibody variable fragment (scFv) or VHH (Nanobody)). In some embodiments, a PTM-specific affinity reagent is an aptamer.

The PTM-specific affinity reagent can specifically bind to an amino acid comprising a phosphorylation (e.g., phospho-tyrosine, phospho-serine, or phospho-threonine), a glycosylation, acetylation (e.g., acetylated lysine), ADP-ribosylation, citrullination, formylation, (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), or ubiquitination (e.g., ubiquitinated lysine).

In some embodiments, the PTM-specific affinity reagent can specifically bind to a phospho-tyrosine, phospho-serine, or phospho-threonine amino acid. In some embodiments, a PTM-specific affinity reagent specifically binds to a serine amino acid comprising a PTM. In some embodiments, a PTM-specific affinity reagent specifically binds to a threonine amino acid comprising a PTM. In some embodiments, a PTM-specific affinity reagent specifically binds to a tyrosine amino acid comprising a PTM. In some embodiments, a PTM-specific affinity reagent specifically binds to a lysine amino acid comprising a PTM. In some embodiments, a PTM-specific affinity reagent specifically binds to an asparagine amino acid comprising a PTM. In some embodiments, a PTM-specific affinity reagent specifically binds to an arginine amino acid comprising a PTM. In some embodiments, a PTM-specific affinity reagent specifically binds to a glycine amino acid comprising a PTM. In some embodiments, a PTM-specific affinity reagent specifically binds to a cysteine amino acid comprising a PTM. In some embodiments, a PTM-specific-affinity reagent specifically binds to a methionine amino acid comprising a PTM. Examples of PTM-specific affinity reagents are described in detail in PCT International Publication No. WO 2025/101639, filed Nov. 6, 2024, the relevant contents of which are incorporated by reference in their entirety.

In some embodiments, PTM recognition comprises monitoring a signal for a series of signal pulses corresponding to binding interactions between the PTM-specific affinity reagent(s) and the polypeptide. In some embodiments, the series of signal pulses provides a characteristic pattern that is representative of the binding interactions and can be used to identify the presence, location, and/or abundance of one or more PTMs in the polypeptide. In some embodiments, a characteristic pattern in the series of signal pulses is indicative of the presence, location, and/or abundance of one or more PTMs in the polypeptide. In some embodiments, a plurality of different characteristic patterns can be determined from a series of signal pulses, where each of the different characteristic patterns is indicative of a different chemical characteristic of the polypeptide (e.g., identity, location, and/or abundance of a particular type of amino acid and/or a particular type of PTM in the polypeptide). Suitable techniques for obtaining such signal pulse information and determining characteristic patterns therein have been described more fully, for example, in PCT International Publication Nos. WO 2020/102741 A1, WO 2021/236983 A2, WO 2023/122769 A2, WO 2024/031031A2, WO 2024/086832A1, WO 2025/101639 A3, and WO 2025/147658 A1, each of which is incorporated by reference in its entirety.

### Polypeptide Analysis

In some aspects, the disclosure provides methods of polypeptide analysis (e.g., polypeptide sequencing). In some embodiments, a method of polypeptide analysis comprises: contacting a polypeptide with a reaction mixture described herein; monitoring a signal for signal pulses corresponding to interactions between one or more amino acid binding proteins and the polypeptide; and determining at least one chemical characteristic of the polypeptide based on a characteristic pattern in the signal.

An association event between an amino acid recognizer and a terminal end of a polypeptide produces a change in magnitude of the signal that persists for a duration of time. Different association events are illustrated for different amino acids exposed at the terminal end of the polypeptide in panels (1-A) and (2-A) of FIG. 1C. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted in FIG. 1C, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, in some embodiments, polypeptide analysis is performed by detecting a series of signal pulses indicative of association of one or more amino acid recognizers with successive amino acids exposed at the terminus of a polypeptide in an ongoing degradation reaction (e.g., over the course of successive terminal amino acid cleavage events in accordance with the disclosure). The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the disclosure are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the disclosure are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the disclosure. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers and one or more modification agents and/or enzymes of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, in accordance with the methods as described herein, amino acid recognizers, which can be used with one or more modification agents and/or enzymes of the disclosure, are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N- or C-terminus) of a polypeptide is immobilized and the other terminus (e.g., the C- or N-terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### Kits

In some aspects, the disclosure provides a kit comprising: one or more modification agents; and one or more enzymes. In some embodiments, the one or more modification agents comprise one or more modification agents described herein. In some embodiments, the one or more enzymes comprise one or more enzymes described herein. In some embodiments, the kit further comprises one or more terminal amino acid recognition molecules. In some embodiments, the kit further comprises one or more PTM-specific affinity reagents. In some embodiments, the kit further comprises instructions for using the kit in a method of polypeptide analysis (e.g., a method of sequencing a polypeptide).

In some embodiments, the one or more modification agents of a kit comprise one or more acetylation agents. In some embodiments, the one or more enzymes of a kit comprise one or more acylpeptide hydrolases. Accordingly, in some embodiments, the kit comprises: one or more acetylation agents; and one or more acylpeptide hydrolases.

In some embodiments, the one or more acetylation agents comprise one or more succinimidyl acetate compounds. In some embodiments, the one or more succinimidyl acetate compounds are selected from N-hydroxysulfosuccinimide acetate, N-hydroxysuccinimide acetate, and sulfosuccinimidyl acetate (Sulfo-NHS acetate). In some embodiments, the one or more succinimidyl acetate compounds comprise Sulfo-NHS acetate.

In some embodiments, the one or more acetylation agents comprise: at least one acyltransferase; and at least one acyl-CoA compound. In some embodiments, an acyltransferase comprises an N-terminal acyltransferase. In some embodiments, the N-terminal acyltransferase is N-myristoyltransferase. In some embodiments, the N-terminal acyltransferase is N-alpha-acetyltransferase. In some embodiments, an acyl-CoA compound is acetyl-CoA.

In some embodiments, the one or more acylpeptide hydrolases comprise an acylpeptide hydrolase described herein. In some embodiments, the one or more acylpeptide hydrolases comprise an acylpeptide hydrolase comprising an amino acid sequence that is at least 80%, at least 85%, at least 90%, at least 95%, at least 97%, 80-100%, 80-90%, 85-95%, 90-99%, 90-100%, 95-99%, 95-100%, 98-100%, or 100% identical to the amino acid sequence of any one of SEQ ID NOs: 1-25. In some embodiments, the one or more acylpeptide hydrolases comprise a human acylpeptide hydrolase, Sus scrofa acylpeptide hydrolase, Aeropyrum pernix acylpeptide hydrolase, Bombyx mori acylpeptide hydrolase, Rattus norvegicus acylpeptide hydrolase, or a homolog or variant thereof.

In some embodiments, the one or more modification agents of a kit comprise one or more handle peptides. In some embodiments, the one or more enzymes of a kit comprise one or more peptidases. Accordingly, in some embodiments, the kit comprises: one or more handle peptides; and one or more peptidases. In some embodiments, a handle peptide comprises or consists of 1, 2, 3, 4, 5, 6, 7, 8, 9, or 10 amino acids in length. In some embodiments, the handle peptide is a dipeptide. In some embodiments, the handle peptide is a tripeptide. In some embodiments, the one or more peptidases comprise a dipeptidyl-peptidase. In some embodiments, the one or more peptidases comprise a tripeptidyl-peptidase. In some embodiments, the handle peptide comprises an acetylated N-terminus or an acetylated C-terminus. In some embodiments, the kit further comprises a deacetylase. In some embodiments, the kit further comprises one or more ligation means for ligating a handle peptide to a terminal amino acid of another polypeptide. In some embodiments, the one or more ligation means comprise a ligase. In some embodiments, the one or more ligation means comprise a subtiligase.

In some embodiments, a kit further comprises one or more terminal amino acid recognition molecules. In some embodiments, the one or more terminal amino acid recognition molecules comprise one or more amino acid binding proteins not having peptide cleavage activity. In some embodiments, each terminal amino acid recognition molecule comprises a detectable label (e.g., a luminescent label, such as a fluorophore dye). In some embodiments, the kit comprises at least two, at least three, at least five, 2-10, 5-10, 5-15, 5-20, or 10-25 terminal amino acid recognition molecules.

In some embodiments, the one or more terminal amino acid recognition molecules comprise one or more amino acid binding proteins. In some embodiments, the one or more amino acid binding proteins are selected from ClpS-homologous proteins, UBR-homologous proteins, Ntaq1-homologous proteins, BIR3 domain-homologous proteins, and variants thereof. Further examples terminal amino acid recognition molecules are described in detail in PCT International Publication No. WO2020/102741A1, filed Nov. 15, 2019, PCT International Publication No. WO2021/236983A2, filed May 20, 2021, PCT International Publication No. WO 2023/122769 A2, filed Dec. 22, 2022, PCT International Publication No. WO 2024/031031 A2, filed Aug. 3, 2023, PCT International Publication No. WO 2024/086832 A1, filed Oct. 20, 2023, PCT International Publication No. WO 2025/101639 A3, filed Nov. 6, 2024, and PCT International Publication No. WO 2025/147658 A1, filed Jan. 3, 2025, the relevant content of each of which is incorporated by reference in its entirety.

In some embodiments, a kit further comprises one or more PTM-specific affinity reagents. In some embodiments, the one or more PTM-specific affinity reagents comprise one or more antibodies that specifically bind to an amino acid comprising a PTM described herein or known in the art. In some embodiments, each PTM-specific affinity reagent comprises a detectable label (e.g., a luminescent label, such as a fluorophore dye). In some embodiments, the kit comprises at least two, at least three, at least five, 2-10, 5-10, 5-15, 5-20, or 10-25 PTM-specific affinity reagents.

In some embodiments, the one or more PTM-specific affinity reagents comprise an affinity reagent that specifically binds to an amino acid comprising a phosphorylated side chain, a glycosylated side chain, an acetylated side chain, an ADP-ribosylated side chain, a citrullinated side chain, a formylated side chain, an N-linked glycosylated side chain, an O-linked glycosylated side chain, a hydroxylated side chain, a methylated side chain, a myristoylated side chain, a neddylated side chain, a nitrated side chain, an oxidated side chain, a palmitoylated side chain, a prenylated side chain, an S-nitrosylated side chain, a sulfated side chain, a sumoylated side chain, or a ubiquitinated side chain. In some embodiments, the one or more PTM-specific affinity reagents comprise an affinity reagent that specifically binds to an amino acid comprising a phosphorylated side chain. In some embodiments, the one or more PTM-specific affinity reagents comprise an affinity reagent that specifically binds to phospho-tyrosine, phospho-serine, or phospho-threonine.

In some embodiments, a kit further comprises instructions for using the kit in a method of polypeptide analysis (e.g., according to a method described herein).

### Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

Distinguishing biological or chemical samples based on fluorophore decay rates and/or fluorophore intensities may provide a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

In some embodiments, a pixel includes a photodetection region, which may be a pinned photodiode (PPD), and a charge storage region, which may be a storage diode (SDO). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of a pixel, excitation light may illuminate a sample well causing incident photons, including fluorescence emissions from a sample, to flow along the optical axis to photodetection region PPD. A pixel may include a waveguide configured to optically (e.g., evanescently) couple excitation light from a grating coupler of the integrated device (not shown) to the sample well. In response, a sample in the sample well may emit fluorescent light toward photodetection region PPD. In some embodiments, a pixel may also include one or more photonic structures, which may include one or more optical rejection structures such as a spectral filter, a polarization filter, and/or a spatial filter. For example, the photonic structures may be configured to reduce the amount of excitation light that reaches the photodetection region and/or increase the amount of fluorescent emissions that reach the photodetection region PPD. A pixel may include one or more metal layers, which may be configured as a filter and/or may carry control signals from a control circuit configured to control transfer gates, as described further herein.

In some embodiments, a pixel may include one or more transfer gates configured to control operation of pixel by applying an electrical bias to one or more semiconductor regions of pixel in response to one or more control signals. For example, when a transfer gate induces a first electrical bias at the semiconductor region between photodetection region and storage region, a transfer path (e.g., charge transfer channel) may be formed in the semiconductor region. Charge carriers (e.g., photo-electrons) generated in photodetection region by the incident photons may flow along the transfer path to storage region. In some embodiments, the first electrical bias may be applied during a collection period during which charge carriers from the sample are selectively directed to storage region. Alternatively, when transfer gate provides a second electrical bias at the semiconductor region between photodetection region and storage region charge carriers from photodetection region may be blocked from reaching storage region along the transfer path. In some embodiments, drain gate REJ may provide a channel to drain to draw noise charge carriers generated in photodetection region by the excitation light away from photodetection region and storage region such as during a rejection period before fluorescent emission photons from the sample reach photodetection region. In some embodiments, during a readout period, transfer gate may provide the second electrical bias and transfer gate may provide an electrical bias to cause charge carriers stored in storage region to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel (e.g., in photodetection region PD) responsive to excitation photons from the light source. For instance, the excitation photons may arrive at pixel prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively, or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### EXAMPLES

### Example 1. Function of Selected Acylpeptide Hydrolases

Acylpeptide hydrolases from Aeropyrum pernix (APH2) and Bombyx mori (APH3) were recombinantly expressed in E. coli cells and purified using affinity chromatography. In addition to wild-type APH2 (referred to as APH2a) and wild-type APH3 (referred to as APH3a), two engineered APH2 proteins were designed and purified—an acylpeptide hydrolase having a deletion of amino acid residues 1-21 (APH2b) and an acylpeptide hydrolase having D15A/R18A substitutions (APH2c).

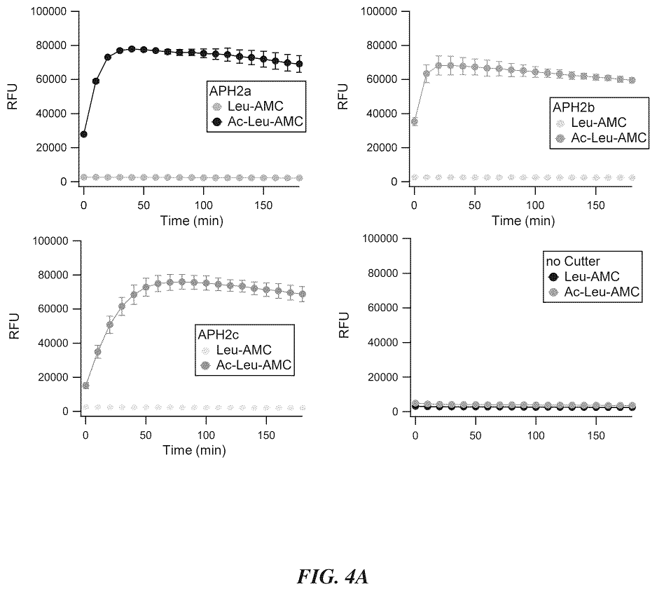

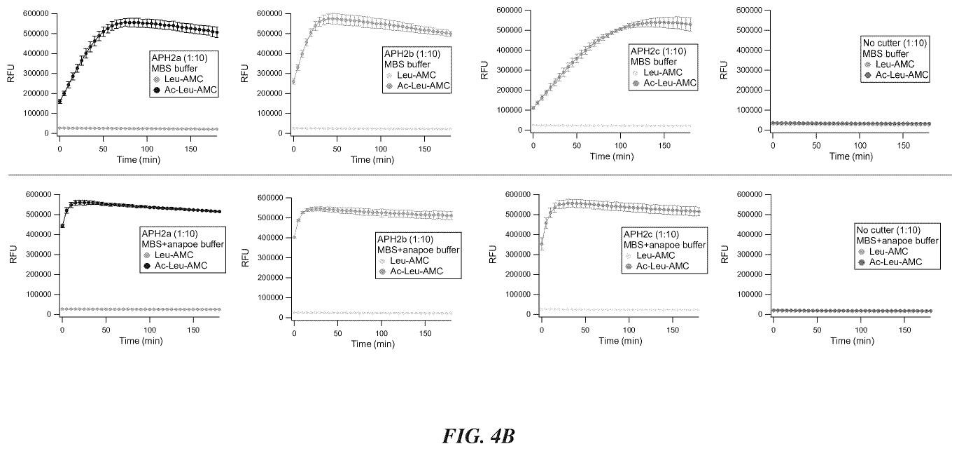

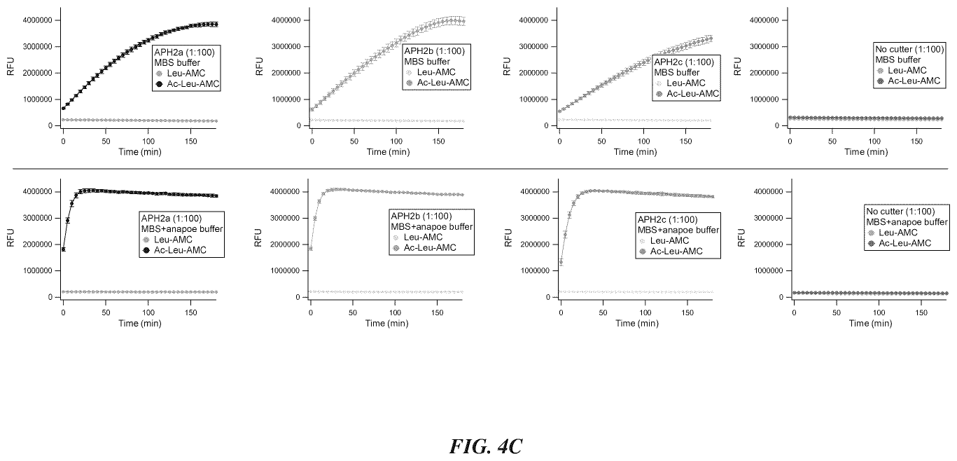

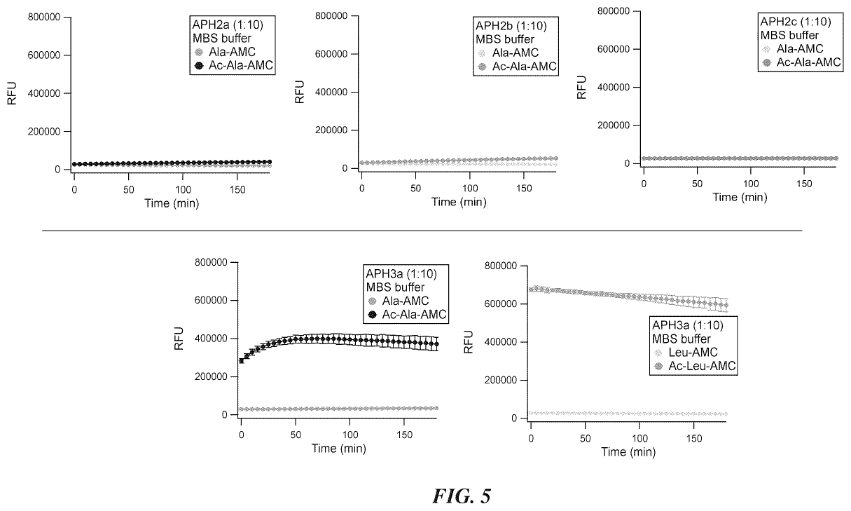

The ability of Aeropyrum pernix acylpeptide hydrolases (APH2a, APH2b, and APH2c) to selectively function in cleavage of an acetylated leucine substrate (Ac-Leu-AMC) relative to an unmodified leucine substrate (Leu-AMC) was determined using bulk activity assays at varying ratios of enzyme-to-substrate concentrations. Control experiments that did not include an acylpeptide hydrolase were also conducted (“no cutter”). The varying ratios of enzyme-to-substrate concentrations were 1:1 enzyme:substrate (500 nM of enzyme, 500 nM of substrate) (FIG. 4A); 1:10 enzyme:substrate (500 nM of enzyme, 5 μM of substrate) (FIG. 4B); and 1:100 enzyme:substrate (500 nM of enzyme, 50 μM of substrate) (FIG. 4C). MBS buffer (50 mM MOPS, 60 mM NaCl, 50 mM Glucose, pH8.0) was used throughout except for the bottom row in FIGS. 4B and 4C, in which MBS buffer in the presence of 0.1% anapoe was used to test the effect of surfactant in the activity of APH.

“AMC” (7-Amino-4-Methylcoumarin) is a fluorophore with excitation/emission at 341/441 nm; and “Ac” denotes acetylation at the N-terminal end of the amino acid. In the assay, enzymatic cleavage of the amino acid by acylpeptide hydrolase results in an increase in fluorescence resulting from the release of the AMC fluorophore. AMC conjugated with an amino acid results in fluorescence quenching and upon enzymatic cleavage of amino acid results in fluorescence dequenching. The AMC substrates were excited at 345 nm wavelength, and the fluorescence emissions were collected at 445 nm wavelength using Agilent BioTek Synergy H1 plate reader. For FIG. 4A, the fluorescence emissions were collected every 10 minutes for 3 hours at 25° C. For FIGS. 4B and 4C, the fluorescence emissions were collected every 5 minutes for 3 hours at 25° C. Each data point indicates the average and the standard deviation of three replicates.

As shown in the data, each of the tested APH2 enzymes demonstrated high levels of catalytic activity with respect to the acetylated leucine substrate, even at low relative concentrations of enzyme (1:100 ratio of enzyme:substrate). Furthermore, none of the tested APH2 enzymes had any activity towards unmodified leucine substrate (i.e., lacking an acetylation). In addition, Leu-AMC and Ac-Leu-AMC substrate in the absence of APH2 showed no indication of dequenching event.

APH2b showed faster cutting activity of Ac-Leu-AMC compared to APH2a, however, APH2c (containing the D15A/R18A double mutant that disrupts salt bridges in the enzyme) showed slower cutting activity compared to APH2a. The non-acetylated substrate, Leu-AMC, showed no indication of cleavage activity during the 3 hours reaction period.

The ability of Aeropyrum pernix acylpeptide hydrolases (APH2a, APH2b, and APH2c) and Bombyx mori acylpeptide hydrolase (APH3a) to selectively function in cleavage of an acetylated alanine substrate (Ac-Ala-AMC) relative to an unmodified alanine substrate (Ala-AMC) was determined using bulk activity assays at a 1:10 ratio of enzyme:substrate (500 nM of enzyme, 5 μM of substrate) in MBS buffer.

As shown in FIG. 5, Bombyx mori acylpeptide hydrolase (APH3a) demonstrated high levels of catalytic/cleavage activity with respect to the acetylated alanine substrate, even at low relative concentration of enzyme (1:10 ratio of enzyme:substrate). Surprisingly, APH3a also demonstrated high catalytic/cleavage activity with respect to the acetylated leucine substrate (Ac-Leu-AMC). Furthermore, Bombyx mori acylpeptide hydrolase did not have any activity towards unmodified alanine or leucine substrates (i.e., lacking an acetylation).

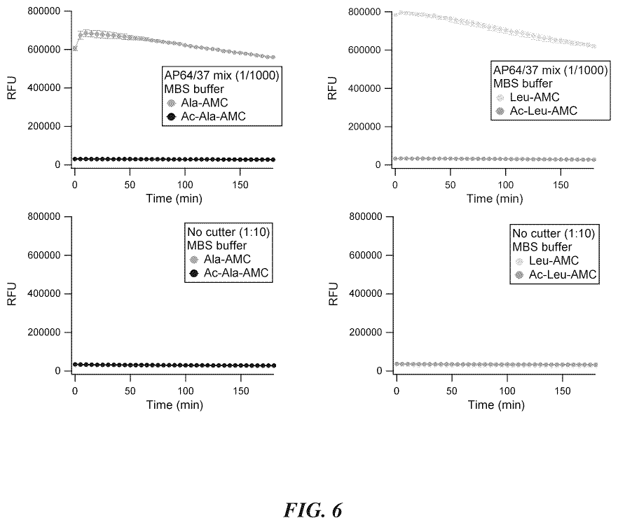

Control experiments using a bulk fluorogenic assay with an aminopeptidase (AP) mixture (AP64 and AP37) to selectively function in cleavage of unmodified alanine (Ala-AMC) and leucine substrate (Leu-AMC) relative to acetylated alanine (Ac-Ala-AMC) and leucine substrate (Ac-Leu-AMC) were performed. It was found that the AP mixture was specific in cleaving nonacetylated amino acid substrates and was not able to cleave acetylated amino acids during the 3-hour time period (FIG. 6). This data demonstrates that the selected aminopeptidase mixture is not capable of cleaving an acetylated peptide. Aminopeptidases and methods of use in polypeptide sequencing, including the aminopeptidases used in this Example (AP64 and AP37), are described more fully in PCT International Publication Nos. WO 2024/086832 A1 and WO 2025/147658 A1, each of which is incorporated herein by reference in its entirety.

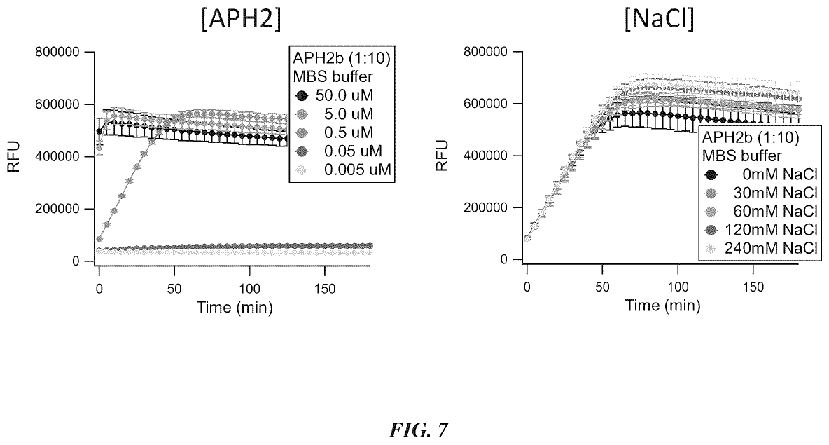

FIG. 7 (left panel) shows a concentration titration of APH2b (0.005, 0.05, 0.5, 5, 50 PM) using Ac-Leu-AMC substrate at 5 μM. FIG. 7 (right panel) shows a concentration titration of NaCl (0, 30, 60, 120, 240 mM) in the MBS buffer using Ac-Leu-AMC substrate. The cleavage activity of APH2b was demonstrated to be concentration dependent with higher activity at higher concentration of the enzyme. However, NaCl concentration did not affect the cleavage activity significantly.

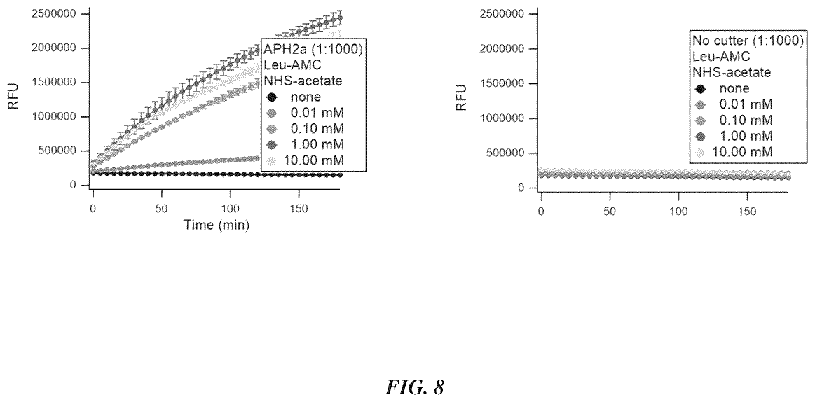

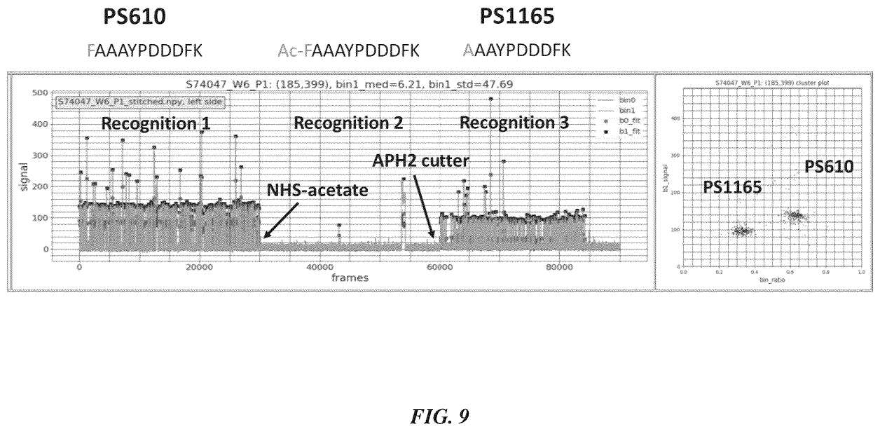

FIG. 8 demonstrates the activation, i.e., acetylation, of non-acetylated Leucine substrate (Leu-AMC) by Sulfo-NHS-acetate at different concentrations (0, 0.01, 0.1, 1, 10 mM) using the MBS buffer consisting of 0.1% anapoe. In the absence of Sulfo-NHS-acetate, no increase in fluorescence is detected. Increasing the concentration of Sulfo-NHS-acetate, increased the cleavage activity of Leu-AMC by APH2a. Sulfo-NHS-acetate at 1 mM showed the optimal cleavage activity of Leu-AMC substrate. Control experiments in the absence of APH2a showed no increase in fluorescence intensity even in the presence of 10 mM NHS-acetate for the 3 hour reaction period.

### Example 2. Controlled Cleavage of Model Peptides

Example on-chip experiments were performed to demonstrate that the methods of the disclosure provide controlled cleavage of peptides immobilized to a surface of a chip. The five experiments described below (on-chip controlled cleavage experiments 1-5) demonstrate that the methods described herein can be utilized to iteratively identify terminal amino acids of a polypeptide in a controlled mechanism. Specifically, modification (e.g., acetylation) of a terminal amino acid of a polypeptide allows for the polypeptide to be contacted with an enzyme (e.g., an acylpeptide hydrolase) that catalyzes the removal of the modified terminal amino acid but not the subsequent terminal amino acid (e.g., the enzyme has increased catalytic activity for removal of the modified terminal amino acid relative to an unmodified terminal amino acid). Amino acid recognition molecules and methods of use in polypeptide sequencing, including the recognition molecules used in these Examples (PS610, PS1165, PS1220, PS1223, and PS1259), are described more fully in PCT International Publication Nos. WO 2021/236983 A2 and WO 2024/031031 A2, each of which is incorporated herein by reference in its entirety.

### On-Chip Controlled Cleavage Experiment 1

FIG. 9 provides a representative controlled cleavage of synthetic peptide QP47 (FAAAYPDDDFK (SEQ ID NO: 26)) using an on-chip recognition assay. Provided in the figure is a trace of single-molecule intensity (signal) versus time (frames). The time resolution was 60 msec per frame for this experiment at 25° C. Three recognition runs were stitched together to produce the full representative trace. The panel on the right shows the correlation between the intensity and the bin ratio of the pulsing activity during recognition of the peptide. Reagent A1 (consisting of five terminal amino acid recognition molecules, each of which specifically recognizes a different amino acid, including PS610 (recognizes phenylalanine) and PS1165 (recognizes alanine)) was used throughout the experiment.

Briefly, the synthetic peptide QP47 (FAAAYPDDDFK (SEQ ID NO: 26)) was loaded (immobilized) on the surface of a chip (POR3.0 H12 chip) using the wash buffer (MBS+0.1% anapoe). Then, Reagent A1 was added for the first recognition step, i.e., Recognition 1 (1-30000 frames) to verify the first amino acid of the peptide, i.e., the phenylalanine in the QP47 sequence. The amino acids are estimated through the intensity, the bin ratio, the pulse width (PW), and the inter pulse duration (IPD) of the pulsing events. After the flow of Reagent 1, cells were rinsed six times using the wash buffer. 10 mM Sulfo-NHS-acetate was then incubated for one hour to acetylate the N-terminal amino acid of loaded peptides. The acetylation was verified by a second recognition step, i.e., Recognition 2 (30001-60000 frames), resulting in acetylated phenylalanine (Ac-FAAAYPDDDFK (SEQ ID NO: 27)). Here the pulsing activity is inhibited due to the modification of the N-terminal amino acid of the peptide. To completely remove the unconjugated Sulfo-NHS-acetate in solution, the flow cells were rinsed extensively with the wash buffer 24 times. Next, acylpeptide hydrolase (APH2a at 33 μM concentration) was injected into the flow cells and incubated for one hour at 25° C. After the incubation, the flow cells were rinsed six times using the wash buffer to remove the APH2a. Reagent A1 was then added to conduct the third recognition run, i.e., Recognition 3 (60001-90000 frames), to verify the cleavage of acetylated phenylalanine (Ac-F) and detect the second amino acid of the loaded peptide, i.e., the first alanine residue in the QP47 sequence. This was confirmed by the signal versus bin ratio cluster plot where the first recognition shows the pulsing activity of PS610 (signal ˜150, bin ratio ˜0.65) and the second recognition shows the pulsing activity of PS1165 (signal ˜100, bin ratio ˜0.35).

### On-Chip Controlled Cleavage Experiment 2

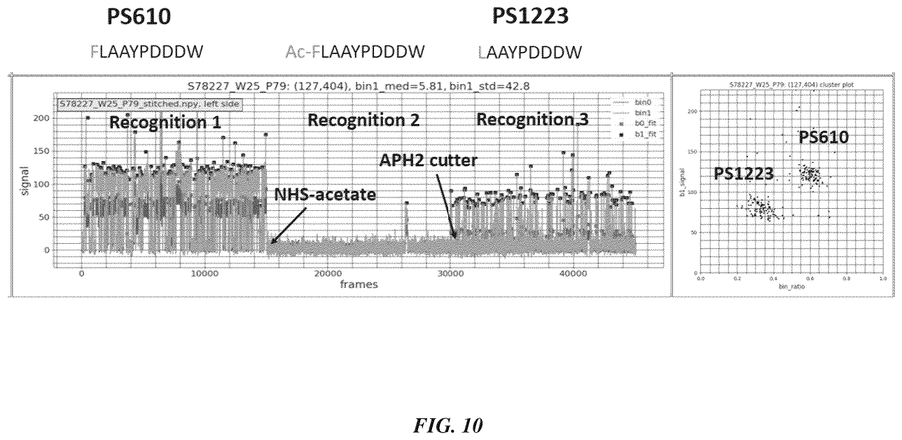

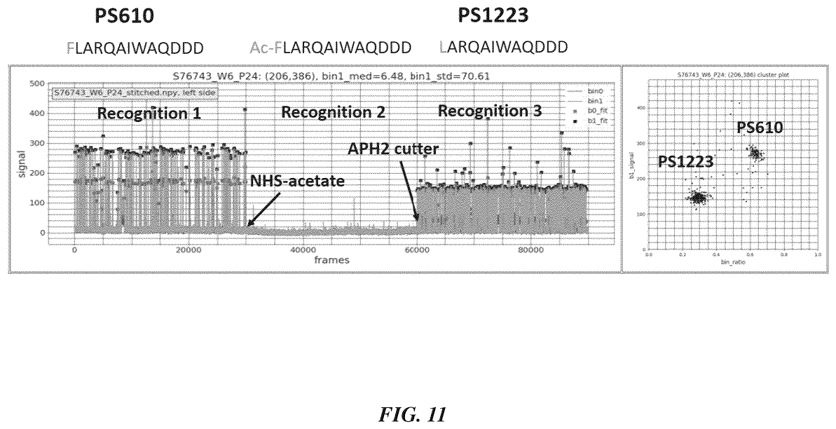

FIG. 10 provides a representative controlled cleavage of synthetic peptide QP649 (FLAAYPDDDW (SEQ ID NO: 29)) using an on-chip recognition assay. Provided in the figure is a trace of single-molecule intensity (signal) versus time (frames). The time resolution was 60 msec per frame for this experiment at 25° C. Three recognition runs were stitched together to produce the full representative trace. The panel on the right shows the correlation between the intensity and the bin ratio of the pulsing activity during recognition of the peptide. Reagent A1 (consisting of five terminal amino acid recognition molecules, each of which specifically recognizes a different amino acid, including PS610 (recognizes phenylalanine) and PS1223 (recognizes leucine)) was used throughout the experiment.

Briefly, the synthetic peptide QP649 (FLAAYPDDDW (SEQ ID NO: 29)) was loaded (immobilized) on the surface of a chip (POR3.0 H12 chip) using the wash buffer (MBS+0.1% anapoe). Then, Reagent A1 was added for the first recognition step, i.e., Recognition 1 (1-30000 frames) to verify the first amino acid of the peptide, i.e., the phenylalanine in the QP649 sequence. The amino acids are estimated through the intensity, the bin ratio, the pulse width (PW), and the inter pulse duration (IPD) of the pulsing events. After the flow of Reagent 1, cells were rinsed six times using the wash buffer. 10 mM Sulfo-NHS-acetate was then incubated for one hour to acetylate the N-terminal amino acid of loaded peptides. The acetylation was verified by a second recognition step, i.e., Recognition 2 (30001-60000 frames), resulting in acetylated phenylalanine (Ac-FLAAYPDDDW (SEQ ID NO: 30)). Here the pulsing activity is inhibited due to the modification of the N-terminal amino acid of the peptide. To completely remove the unconjugated Sulfo-NHS-acetate in solution, the flow cells were rinsed extensively with the wash buffer 24 times. Next, acylpeptide hydrolase (APH2a at 31.3 μM concentration) was injected into the flow cells and incubated for one hour at 25° C. After the incubation, flow cells were rinsed six times using the wash buffer to remove the APH2a. Reagent A1 was then added to conduct the third recognition run, i.e., Recognition 3 (60001-90000 frames), to verify the cleavage of acetylated phenylalanine (Ac-F) and detect the second amino acid of the loaded peptide, i.e., the first leucine residue in the QP649 sequence. This was confirmed by the signal versus bin ratio cluster plot where the first recognition shows the pulsing activity of PS610 (signal ˜125, bin ratio ˜0.65) and the second recognition shows the pulsing activity of PS1223 (signal ˜80, bin ratio ˜0.35).

### On-Chip Controlled Cleavage Experiment 3

FIG. 11 provides a representative controlled cleavage of synthetic peptide QP1160 (FLARQAIWAQDDD (SEQ ID NO: 32)) using an on-chip recognition assay. Provided in the figure is a trace of single-molecule intensity (signal) versus time (frames). The time resolution was 60 msec per frame for this experiment at 25° C. Three recognition runs were stitched together to produce the full representative trace. The panel on the right shows the correlation between the intensity and the bin ratio of the pulsing activity during recognition of the peptide. Reagent A2 (consisting of six terminal amino acid recognition molecules, each of which specifically recognizes a different amino acid, including PS610 (recognizes phenylalanine) and PS1223 (recognizes leucine)) was used throughout the experiment.

Briefly, the synthetic peptide QP1160 (FLARQAIWAQDDD (SEQ ID NO: 32)) was loaded (immobilized) on the surface of a chip (POR3.0 H12 chip) using the wash buffer (MBS+0.1% anapoe). Then, Reagent A2 was added for the first recognition step, i.e., Recognition 1 (1-30000 frames) to verify the first amino acid of the peptide, i.e., the phenylalanine in the QP1160 sequence. The amino acids are estimated through the intensity, the bin ratio, the pulse width (PW), and the inter pulse duration (IPD) of the pulsing events. After the flow of Reagent 2, cells were rinsed six times using the wash buffer. 10 mM Sulfo-NHS-acetate was then incubated for one hour to acetylate the N-terminal amino acid of loaded peptides. The acetylation was verified by a second recognition step, i.e., Recognition 2 (30001-60000 frames), resulting in acetylated phenylalanine (Ac-FLARQAIWAQDDD (SEQ ID NO: 33)). Here the pulsing activity is inhibited due to the modification of the N-terminal amine of the peptide. To completely remove the unconjugated Sulfo-NHS-acetate in solution, the flow cells were rinsed extensively with the wash buffer 24 times. Next, acylpeptide hydrolase (APH2a at 5.5 μM concentration) was injected into the flow cells and incubated for one hour at 40° C. Note the temperature was increased to 40° C. only during the cleavage step. After the incubation, flow cells were rinsed six times using the wash buffer. Reagent A2 was then added to conduct the third recognition run, i.e., Recognition 3 (60001-90000 frames), to verify the cleavage of acetylated phenylalanine (Ac-F) and detect the second amino acid of the loaded peptide, i.e., the first leucine residue in the QP1160 sequence. This was confirmed by the signal versus bin ratio cluster plot where the first recognition shows the pulsing activity of PS610 (signal ˜265, bin ratio ˜0.65) and the second recognition shows the pulsing activity of PS1223 (signal ˜150, bin ratio ˜0.3).

### On-Chip Controlled Cleavage Experiment 4

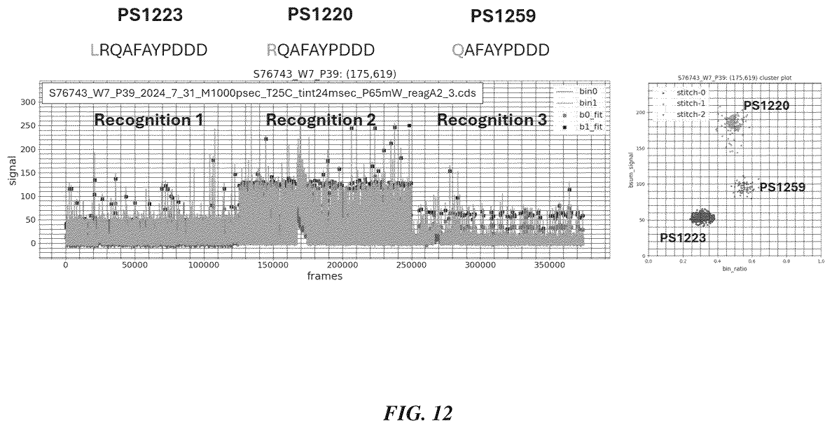

FIG. 12 provides a representative controlled cleavage of synthetic peptide QP941 (LRQAFAYPDDD (SEQ ID NO: 35)) using an on-chip recognition assay. Provided in the figure is a trace of single-molecule intensity (signal) versus time (frames). The time resolution was 24 msec per frame for this experiment at 25° C. Three recognition runs were stitched together to produce the full representative trace. The panel on the right shows the correlation between the intensity and the bin ratio of the pulsing activity during recognition of the peptide. Reagent A2 (consisting of six terminal amino acid recognition molecules, each of which specifically recognizes a different amino acid, including PS1223 (recognizes leucine), PS1220 (recognizes arginine), and PS1259 (recognizes glutamine)) was used throughout the experiment.

Briefly, the synthetic peptide QP941 (LRQAFAYPDDD (SEQ ID NO: 35)) was loaded (immobilized) on the surface of a chip (POR3.0 H12 chip) using the wash buffer (MBS+0.1% anapoe). Then, Reagent A2 was added for the first recognition step, i.e., Recognition 1 (1-125000 frames) to verify the first amino acid of the peptide, i.e., the leucine in the QP941 sequence. The amino acids are estimated through the intensity, the bin ratio, the pulse width (PW), and the inter pulse duration (IPD) of the pulsing events. After the flow of Reagent 2, cells were rinsed six times using the wash buffer. 10 mM Sulfo-NHS-acetate was then incubated for one hour to acetylate the N-terminal amino acid of loaded peptides. To completely remove the unconjugated Sulfo-NHS-acetate in solution, the flow cells were rinsed extensively with the wash buffer 24 times. Next, acylpeptide hydrolase (APH2b at 400 μM concentration) was injected into the flow cells and incubated for 90 min at 45° C. Note the temperature was increased to 45° C. only during the cleavage step. After the incubation, flow cells were rinsed six times using the wash buffer. Reagent A2 was then added to conduct the second recognition run, i.e., Recognition 2 (125001-250000 frames), to verify the cleavage of acetylated leucine (Ac-L) and detect the second amino acid of the loaded peptide, i.e., the first arginine residue in the QP941 sequence. To detect the third amino acid of QP941, acetylation and the cleavage step was repeated using the same condition indicated above. Reagent A2 was then added to conduct the third recognition run, i.e., Recognition 3 (250001-375000 frames), to verify the cleavage of acetylated arginine (Ac-R) and detect the third amino acid of the loaded peptide, i.e., the first glutamine residue in the QP941 sequence. Recognition was confirmed by the signal versus bin ratio cluster plot where the first recognition (colored in blue) shows the pulsing activity of PS1223 (signal ˜50, bin ratio ˜0.3), the second recognition (colored in red) shows the pulsing activity of PS1220 (signal ˜180, bin ratio ˜0.5), and the third recognition (colored in green) shows the pulsing activity of PS1259 (signal ˜100, bin ratio ˜0.55).

### On-Chip Controlled Cleavage Experiment 5

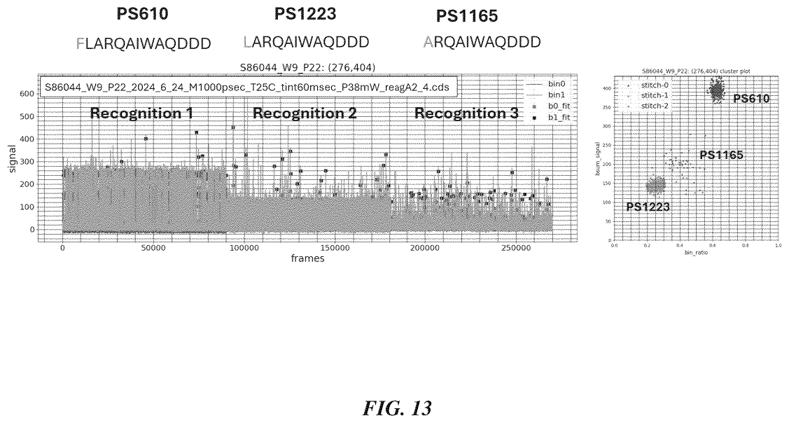

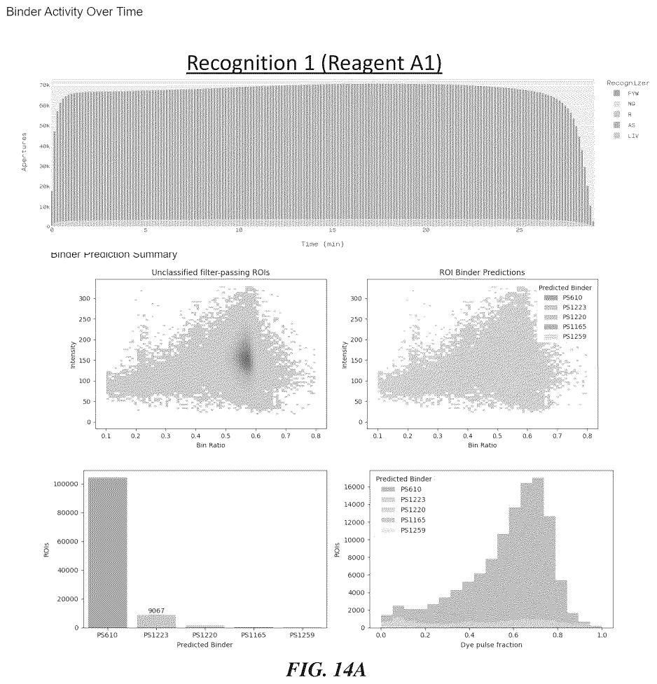

FIG. 13 provides a representative controlled cleavage of synthetic peptide QP1160 (FLARQAIWAQDDD (SEQ ID NO: 32)) using an on-chip recognition assay. Provided in the figure is a trace of single-molecule intensity (signal) versus time (frames). The time resolution was 60 msec per frame for this experiment at 25° C. Three recognition runs were stitched together to produce the full representative trace. The panel on the right shows the correlation between the intensity and the bin ratio of the pulsing activity during recognition of the peptide. Reagent A2 (consisting of six terminal amino acid recognition molecules, each of which specifically recognizes a different amino acid, including PS610 (recognizes phenylalanine), PS1223 (recognizes leucine), and PS1165 (alanine glutamine)) was used throughout the experiment.

Briefly, the synthetic peptide QP1160 (FLARQAIWAQDDD (SEQ ID NO: 32)) was loaded (immobilized) on the surface of a chip (POR3.0 H12 chip) using the wash buffer (MBS+0.1% anapoe). Then, Reagent A2 was added for the first recognition step, i.e., Recognition 1 (1-90000 frames) to verify the first amino acid of the peptide, i.e., the phenylalanine in the QP1160 sequence. The amino acids are estimated through the intensity, the bin ratio, the pulse width (PW), and the inter pulse duration (IPD) of the pulsing events. After the flow of Reagent 2, cells were rinsed six times using the wash buffer. 10 mM Sulfo-NHS-acetate was then incubated for one hour to acetylate the N-terminal amino acid of loaded peptides. To completely remove the unconjugated Sulfo-NHS-acetate in solution, the flow cells were rinsed extensively with the wash buffer 24 times. Next, acylpeptide hydrolase (APH2b at 40 μM concentration) was injected into the flow cells and incubated for 90 min at 45° C. Note the temperature was increased to 45° C. only during the cleavage step. After the incubation, flow cells were rinsed six times using the wash buffer. Reagent A2 was then added to conduct the second recognition run, i.e., Recognition 2 (90001-180000 frames), to verify the cleavage of acetylated phenylalanine (Ac-F) and detect the second amino acid of the loaded peptide, i.e., the first leucine residue in the QP1160 sequence. To detect the third amino acid of QP1160, acetylation and the cleavage step was repeated using the same condition indicated above. Reagent A2 was then added to conduct the third recognition run, i.e., Recognition 3 (180001-270000 frames), to verify the cleavage of acetylated leucine (Ac-L) and detect the third amino acid of the loaded peptide, i.e., the first alanine residue in the QP1160 sequence. Recognition was confirmed by the signal versus bin ratio cluster plot where the first recognition (colored in blue) shows the pulsing activity of PS610 (signal ˜400, bin ratio ˜0.6), the second recognition (colored in red) shows the pulsing activity of PS1223 (signal ˜150, bin ratio ˜0.25), and the third recognition (colored in green) shows the pulsing activity of PS1165 (signal ˜200, bin ratio ˜0.4).

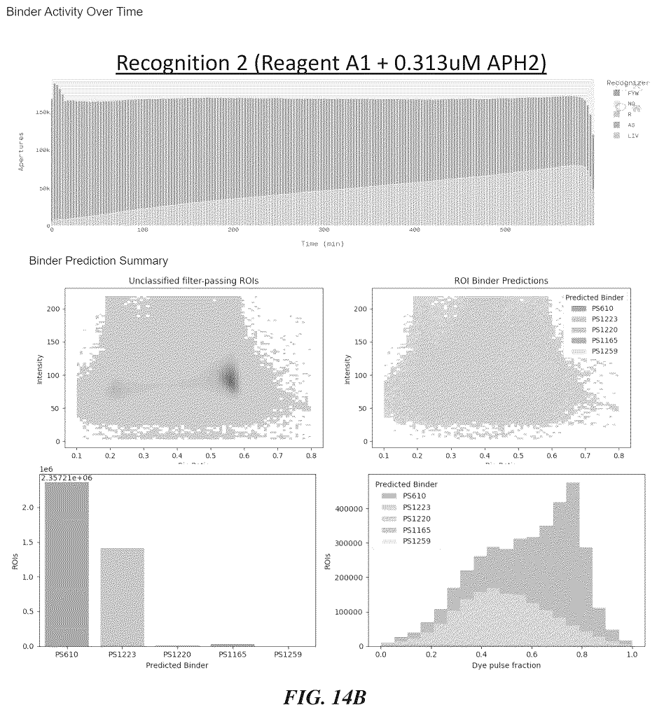

FIGS. 14A-14B provide example results from an on-chip cloud-based recognition assay. The workflow consisted of three steps, as provided below:
Step 1: 30 min recognition of synthetic peptide (e.g., QP649) using Reagent A1 (consisting of five terminal amino acid recognition molecules, each of which specifically recognizes a different amino acid).
Step 2: Acetylation of the N-terminal amino acid using Sulfo-NHS-acetate at 10 mM.
Step 3: 10-hour recognition of acetylated synthetic peptide (e.g., acetylated QP649) using Reagent A1 in the presence of acylpeptide hydrolase (e.g., APH2a).

The graphs in FIG. 14A show the cloud-based analysis of Recognition 1 in step 1 in the absence of APH2a, and the graphs in FIG. 14B show the cloud-based analysis of Recognition 2 in step 3 in the presence of APH2a. The gradual increase of the orange histogram in the Binder Activity Over Time graph (top panel of FIG. 14B) indicates the cleavage activity of APH2a. In addition, the cloud-based analysis confirmed the detection of leucine residue by recognizer PS1223, which is the 2^{nd} residue of QP649, shown in the Binder Prediction Summary panels.

### Example 3. Development of Engineered Acylpeptide Hydrolase Variants

Example 1 describes the design and evaluation of two engineered variants of acylpeptide hydrolase from Aeropyrum pernix (APH2): an N-terminal truncation variant having a deletion of amino acid residues 1-21 (APH2b), and a variant having D15A and R18A substitutions (APH2c) relative to wild-type APH2 (APH2a). This Examples describes the further design and evaluation of truncated and substituted variants of APH2.

### APH2 N-Terminal Truncation Constructs

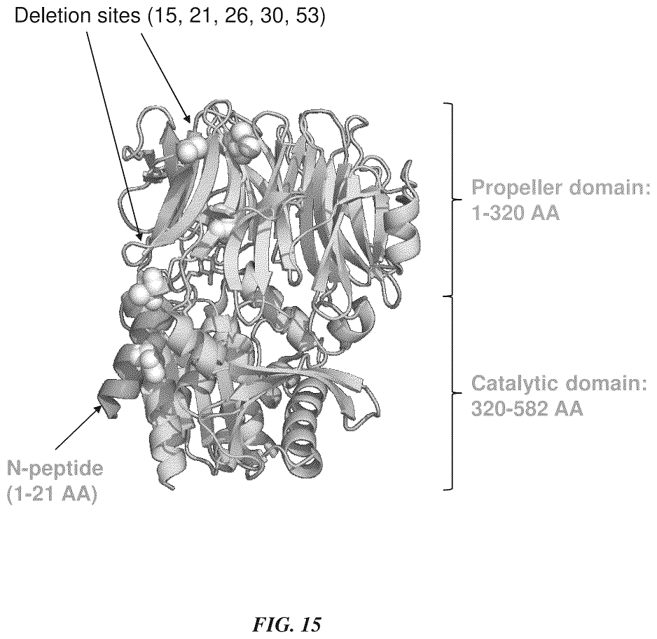

FIG. 15 depicts the structure of APH2 (based on PDB: 1VE6). As shown, APH2 includes an N-terminal domain (“propeller domain”) and a C-terminal catalytic domain. Five N-terminal truncation variants were designed by identifying specific deletion sites in the N-terminal domain. These truncation variants, which are summarized below in Table 2, were expressed and purified as described in Example 1.

TABLE 2
N-terminal Truncation Variants of APH2

| Name | Truncation relative to APH2a (SEQ ID NO: 1) | SEQ ID NO |
|---|---|---|
| APH2b | Deletion of amino acids through position 21 | 2 |
| APH2d15 | Deletion of amino acids through position 15 | 13 |
| APH2d26 | Deletion of amino acids through position 26 | 14 |
| APH2d30 | Deletion of amino acids through position 30 | 15 |
| APH2d53 | Deletion of amino acids through position 53 | 16 |

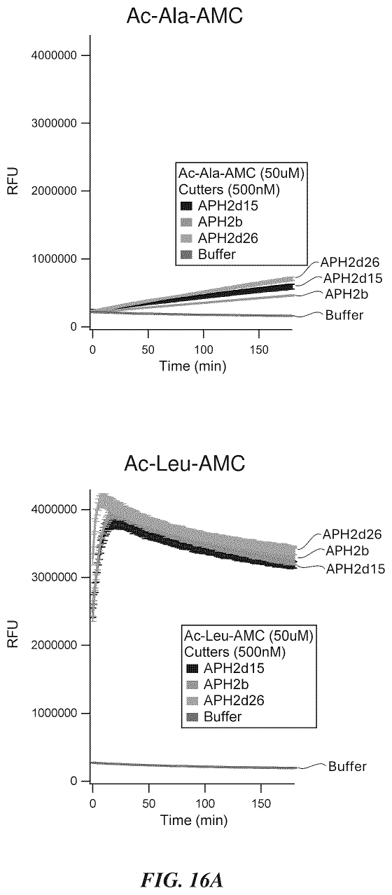

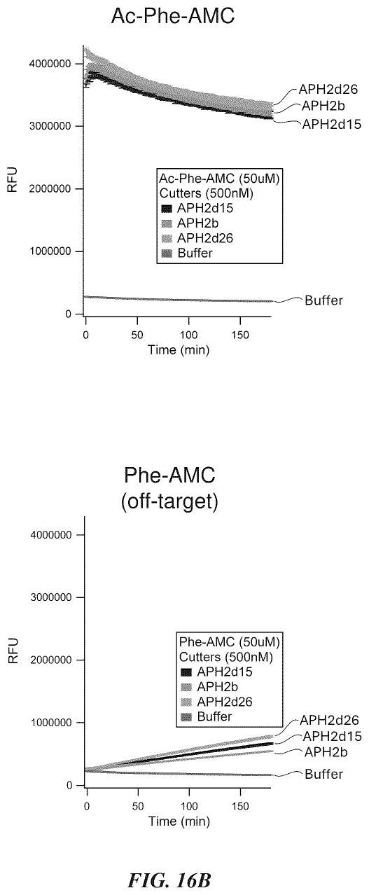

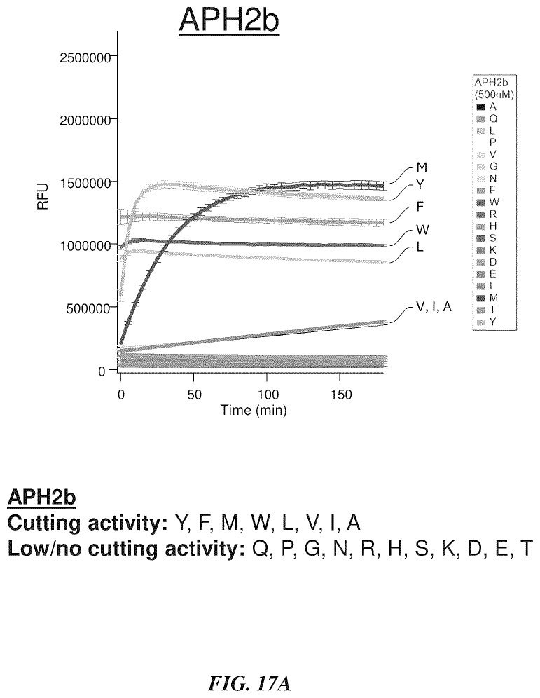

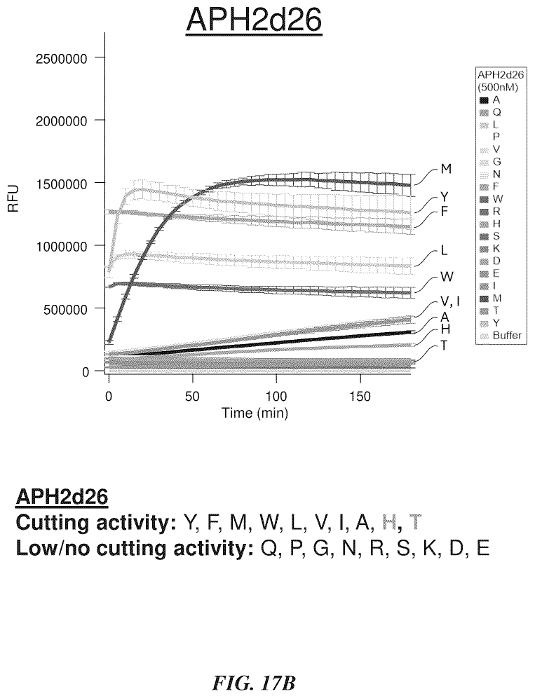

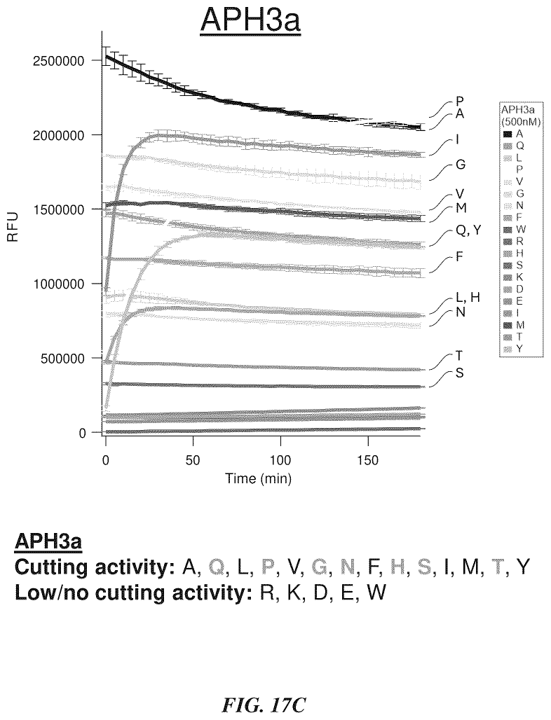

The cleavage activity of the N-terminal truncation variants was evaluated in bulk fluorogenic cutting activity assays using AMC-conjugated amino acid substrates as described in Example 1. Selected results are shown in FIGS. 16A-16B for APH2d15, APH2b, and APH2d26 in cleavage assays with acetylated alanine substrate (FIG. 16A, top plot), acetylated leucine substrate (FIG. 16A, bottom plot), acetylated phenylalanine substrate (FIG. 16B, top plot), and unmodified phenylalanine substrate (FIG. 16B, bottom plot). Further selected results using 19 different acetylated amino acid substrates are shown in FIGS. 17A-17C for APH2b (FIG. 17A), APH2d26 (FIG. 17B), and APH3a (FIG. 17C). In each assay, enzyme was present at a concentration of 500 nM, and substrate was present at a concentration of 50 μM.

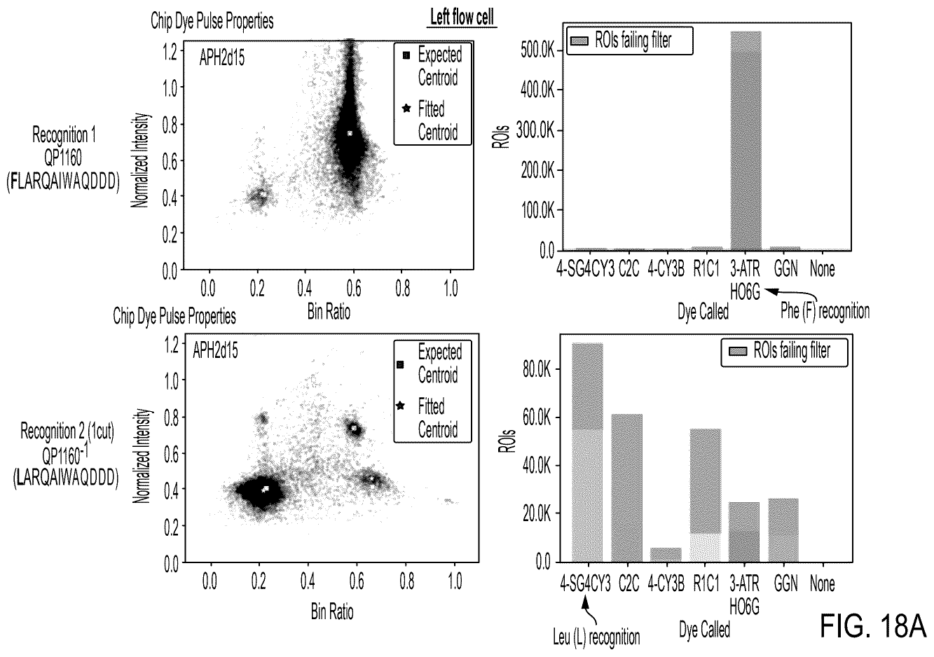

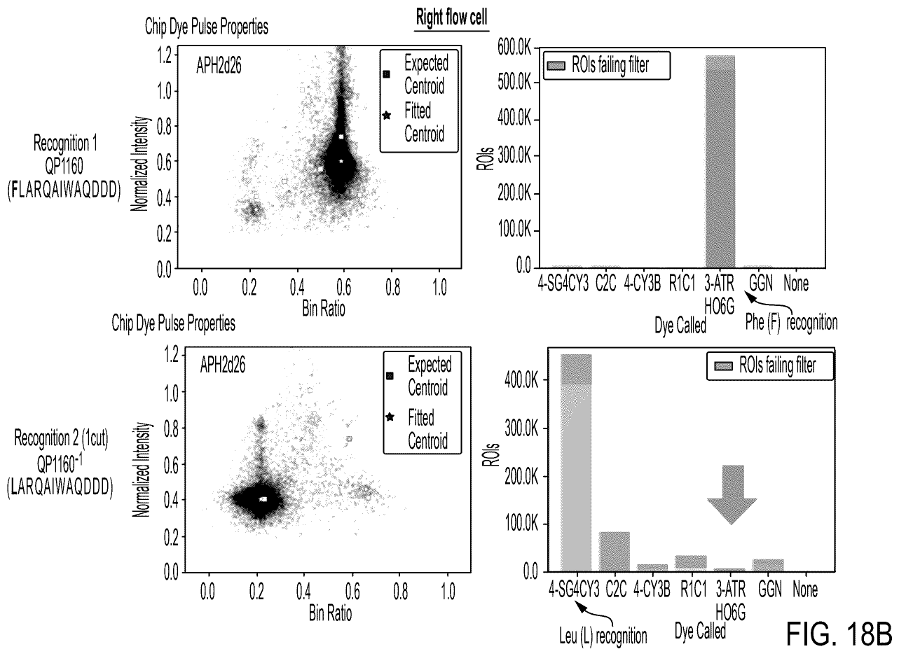

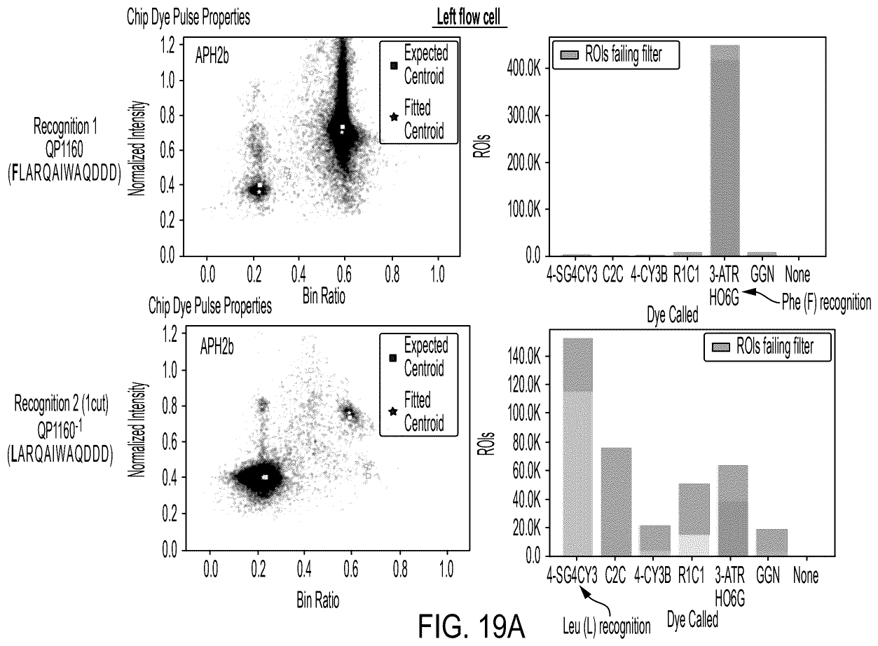

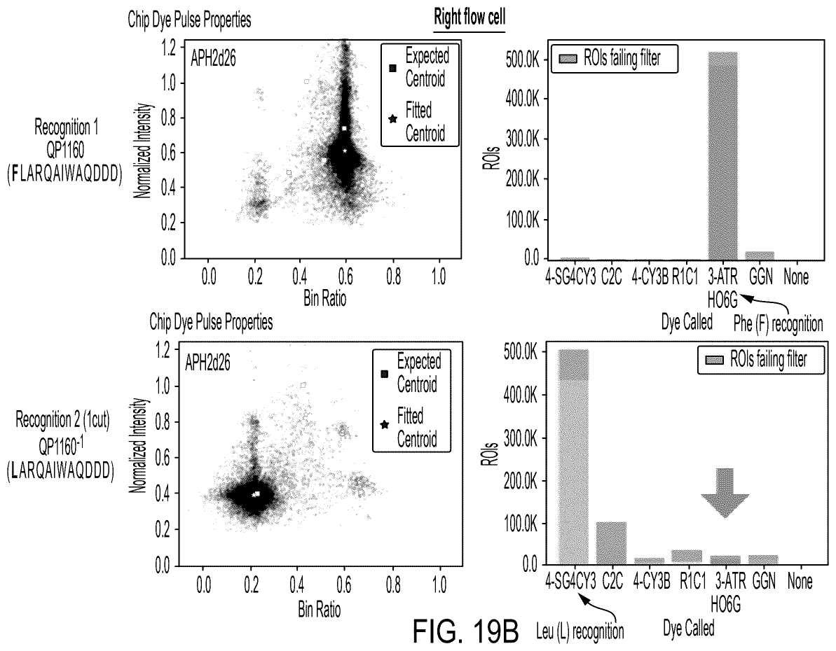

On-chip controlled cleavage runs with N-terminal truncation variants were performed as described in Example 2 using the synthetic peptide QP1160 (FLARQAIWAQDDD (SEQ ID NO: 32)). Selected results are provided in FIGS. 18A-18B, showing on-chip results carried out with APH2d15 in the left flow cell (FIG. 18A) and APH2d26 in the right flow cell (FIG. 18B), and in FIGS. 19A-19B, showing on-chip results carried out with APH2b in the left flow cell (FIG. 19A) and APH2d26 in the right flow cell (FIG. 19B). In each of FIGS. 18A-18B and 19A-19B, results are shown for recognition steps before (Recognition 1, top plots) and after (Recognition 2, bottom plots) acetylation and APH-catalyzed cleavage of N-terminal phenylalanine (F).

As shown in FIGS. 18A-18B, Recognition 1 accurately identified the N-terminal F of QP1160. Following acetylation and incubation with APH2d15 or APH2d26, Recognition 2 accurately identified N-terminal L, indicating that each enzyme successfully cleaved the acetylated N-terminal F. Additionally, comparing the bottom right plots of FIGS. 18A-18B, APH2d26 showed significantly increased cutting activity compared to APH2d15, with cleavage by APH2d26 resulting in an increase in L recognition and a decrease in residual F detection in Recognition 2. Similarly, in the runs performed with APH2b and APH2d26 in the left and right flow cells, respectively, Recognition 2 accurately identified N-terminal L, indicating that each enzyme successfully cleaved the acetylated N-terminal F (FIGS. 19A-19B). Comparing the bottom right plots of FIGS. 19A-19B, APH2d26 again showed significantly increased cutting activity by the relative increase in L recognition and decrease in residual F detection in Recognition 2.

As shown in the representative experimental results, each of the tested APH2 N-terminal truncation variants demonstrated high levels of cutting activity toward acetylated terminal amino acid substrates. The 26-residue truncation variant, APH2d26, showed significantly increased cutting activity compared to other enzymes as demonstrated by both on- and off-chip activity assays. Additionally, compared to the 21-residue truncation variant (APH2b), APH2d26 is further capable of cutting acetylated histidine and acetylated threonine in bulk activity assay.

APH2b Variants with Off-Target Mutations

Nine variants of APH2b having amino acid substitutions were designed and evaluated for decreasing off-target cutting activity. As described previously, APH2b is an N-terminal truncation variant in which amino acids through position 21 relative to APH2a (SEQ ID NO: 1) are deleted. The nine variants described here are further modified by amino acid substitutions relative to APH2a, as shown below in Table 3 (substitutions relative to position numbering in APH2a (SEQ ID NO: 1)). Each variant was expressed and purified as described in Example 1.

TABLE 3
Substituted Variants of APH2b

| | Substitution(s) relative to | |
|---|---|---|
| Name | APH2a (SEQ ID NO: 1) | SEQ ID NO |
| APH2b-A (“A”) | F485L | 17 |
| APH2b-B (“B”) | F485L, F488V | 18 |
| APH2b-C (“C”) | F485L, F488M | 19 |
| APH2b-D (“D”) | F485L, L492I | 20 |
| APH2b-E (“E”) | R113Y, F485L, F488L | 21 |
| APH2b-F (“F”) | R113N, F485M, F488L | 22 |
| APH2b-G (“G”) | V471T | 23 |
| APH2b-H (“H”) | V471S | 24 |
| APH2b-I (“I”) | V471I, F485L, F488L, T527S | 25 |

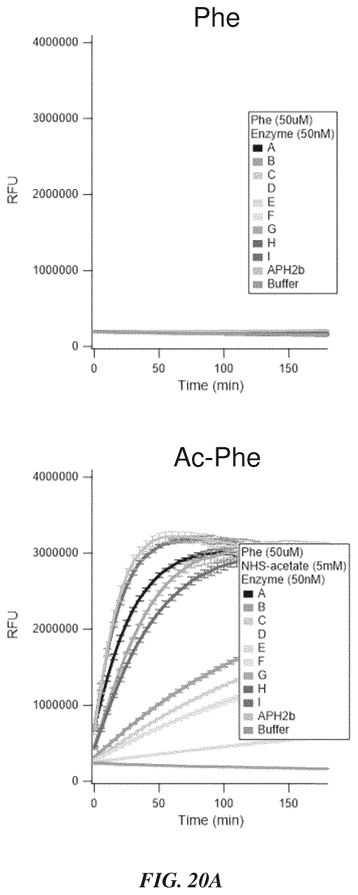

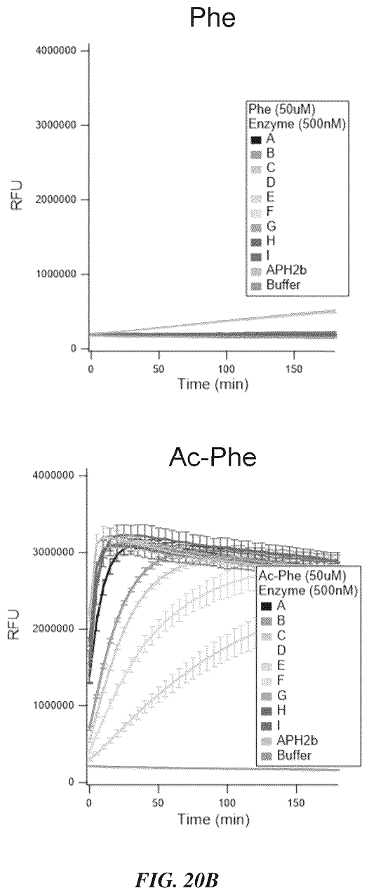

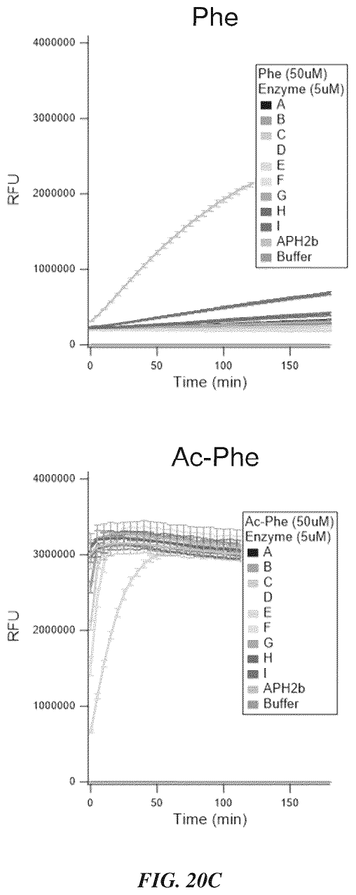

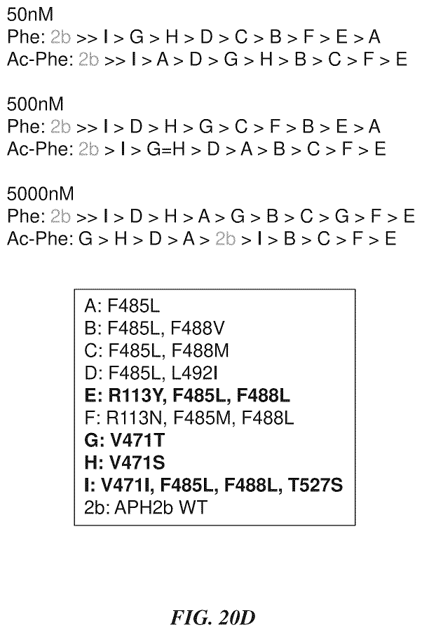

The cleavage activity of the APH2b variants was evaluated in bulk fluorogenic cutting activity assays using AMC-conjugated amino acid substrates as described previously. Example results are shown in FIGS. 20A-20C for cleavage assays carried out at enzyme concentrations of 50 nM (FIG. 20A), 500 nM (FIG. 20B), and 5 μM (FIG. 20C) with unmodified phenylalanine substrate (Phe) and acetylated phenylalanine substrate (Ac-Phe). Results are shown in each plot with labels A-I corresponding to substituted variants as defined in Table 3. FIG. 20D provides a summary ranking of the activity levels from the results shown in FIGS. 20A-20C. Variants G, H, and I were selected for further evaluation based on the activity levels toward acetylated substrates being similar to or greater than the activity of APH2b at the different enzyme concentrations (50, 500, 5000 nM). The variant E was selected as a negative control for the experiments described below.

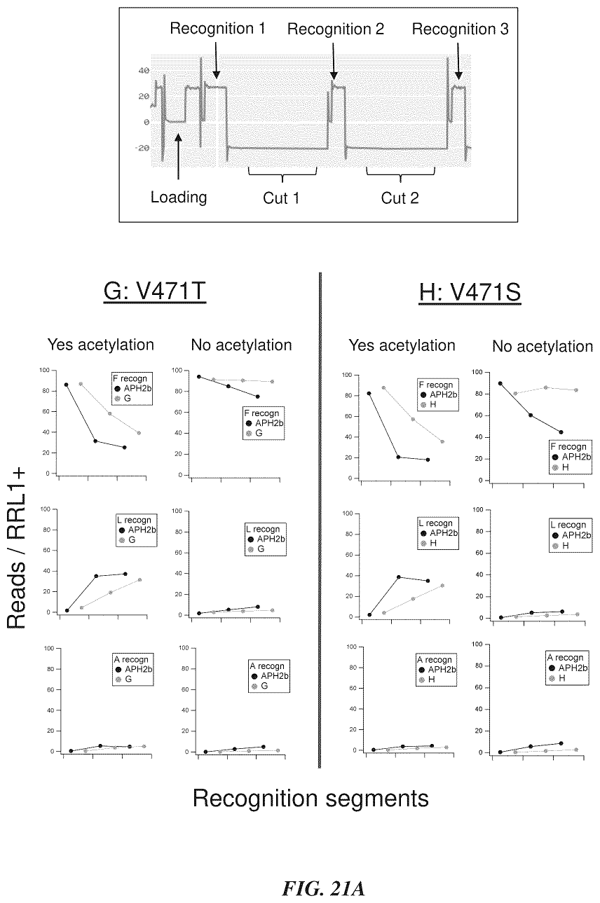

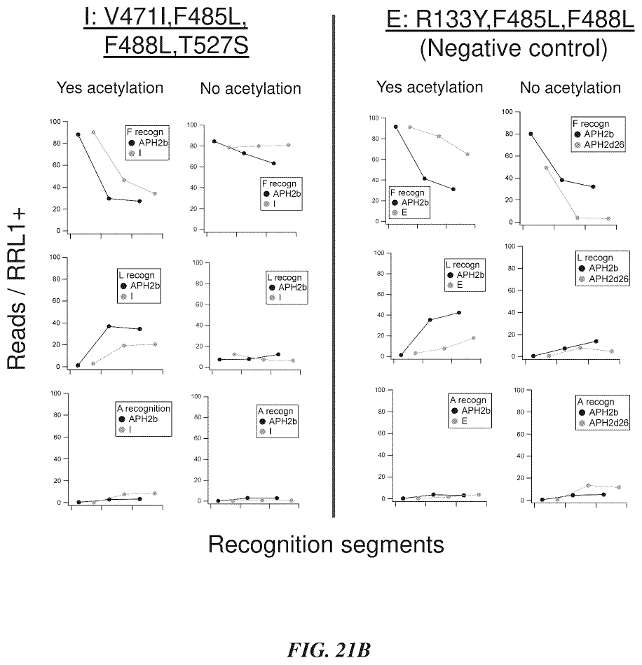

The cleavage activities of APH2b-G (“G”), APH2b-H (“H”), and APH2b-I (“I”) were further evaluated in on-chip cutting activity assays using the synthetic peptide QP1160 (FLARQAIWAQDDD (SEQ ID NO: 32)). The variants were evaluated alongside APH2b or APH2d26, with APH2b-E (“E”) serving as a negative control. Example results are shown in FIGS. 21A-21B, and the panel at the top of FIG. 21A provides a general workflow illustrating how these experiments were performed. The assay runs included three 30-minute recognition runs at 60 ms integration time after the peptide is loaded. For each recognition run, a mixture of labeled amino acid recognizers was added and recognition signal monitored. In between Recognition 1-2 and Recognition 2-3, enzyme was added with (“Yes acetylation”) or without (“No acetylation”) a preceding step of acetylation.

The plots in FIGS. 21A-21B show the number of reads normalized by the number of apertures with RRL1+ (recognizer-homopolymer collapsed read length of 1 or greater), and Recognition segments indicate the number of reads detected for the first three amino acids of QP1160 peptide (F, L, A) using the core analysis for the three 30-minute recognition runs. By way of example, the results for variant G (V471T) compared alongside APH2b are shown in the plots at left in FIG. 21A. The data points in the top panels show the recognition of residue “F” in each 30-minute recognition run (Recognition 1-3 corresponding to points 1-3 along the x-axis). The decrease in the percentage of Reads/RRL1+ from one recognition run to the next indicates cleavage activity. The middle panels show the recognition of the second residue “L,” and the bottom panels show the recognition of the third residue “A.”

These results demonstrated that, without acetylation, the variants G, H, I showed no significant off-target cleavage as compared to wild-type enzyme.

### Example 4. Representative Workflow for Automated Peptide Sequencing

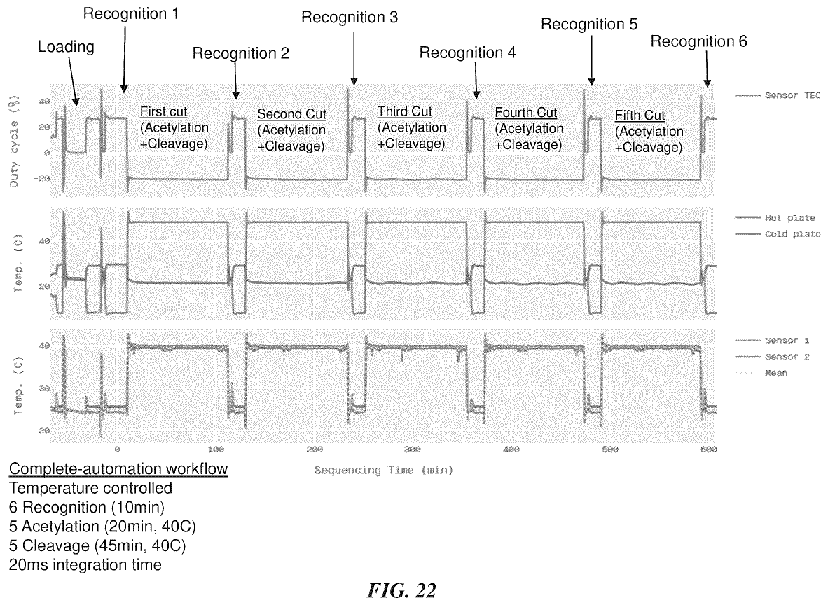

FIG. 22 depicts a representative automation workflow for peptide sequencing. As shown, the workflow involved five steps of controlled cleavage and six recognition runs. The complete automation workflow is outlined below.
i) MBS buffer (50 mM MOPS, 60 mM NaCl pH 8, 50 mM Glucose, 0.1% anapoe) used to wet the chip;
ii) Peptide is loaded;
iii) Recognition 1: 10-minute recognition using amino acid recognizer mix;
iv) Temperature increased to 45° C.;
v) Acetylation step: 20 mM NHS-acetate added to acetylate the peptides for 20 minutes;
vi) Cleavage step: APH enzymes (400 μM monomer concentration) added and incubated for 45 minutes;
vii) Temperature dropped to 25° C.;
viii) Recognition 2: 10-minute recognition using amino acid recognizer mix;
ix) Repeat steps iv-vii;
x) Recognition 3: 10-minute recognition using amino acid recognizer mix;
xi) Repeat steps iv-vii;
xii) Recognition 4: 10-minute recognition using amino acid recognizer mix;
xiii) Repeat steps iv-vii;
xiv) Recognition 5: 10-minute recognition using amino acid recognizer mix;
xv) Repeat steps iv-vii;
xvi) Recognition 6: 10-minute recognition using amino acid recognizer mix.

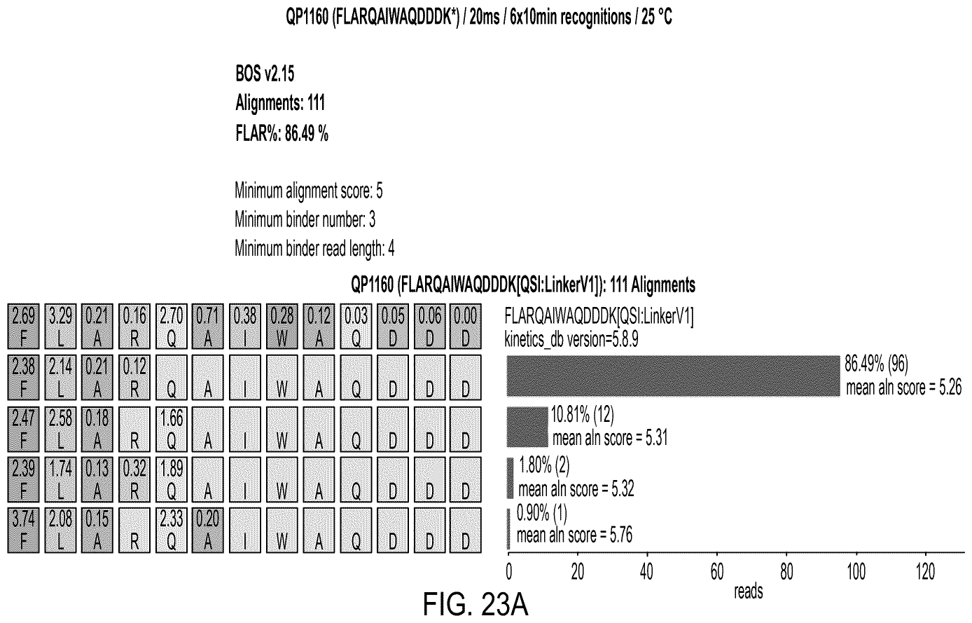

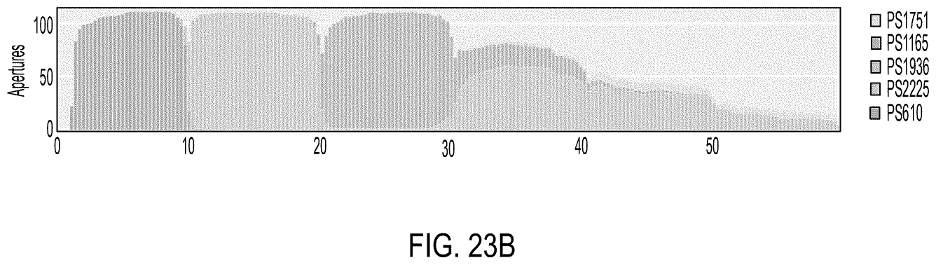

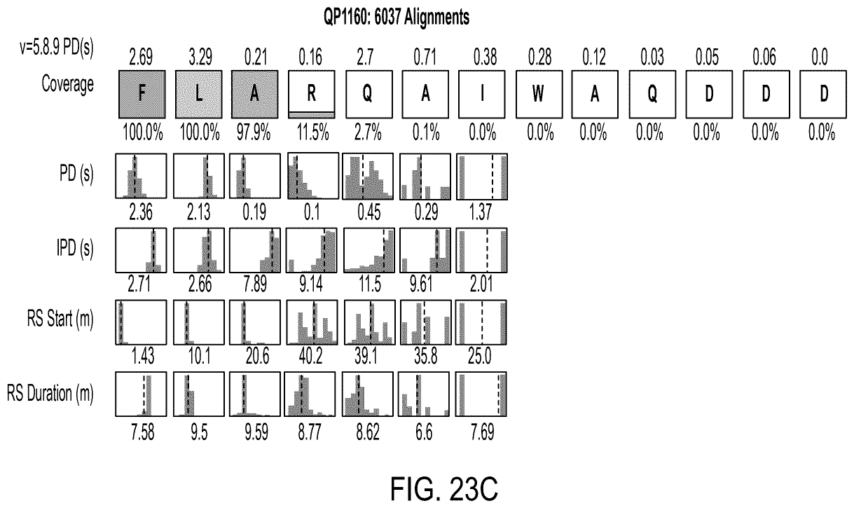

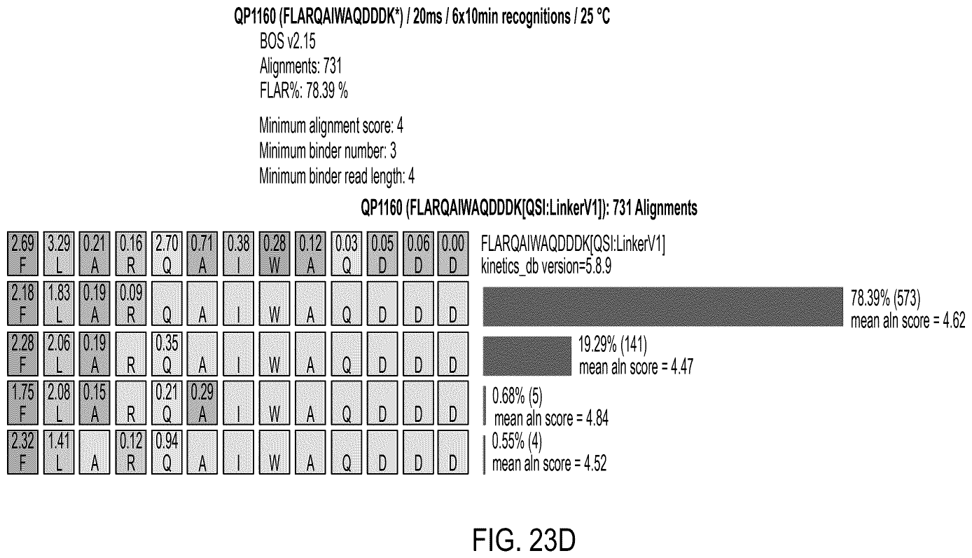

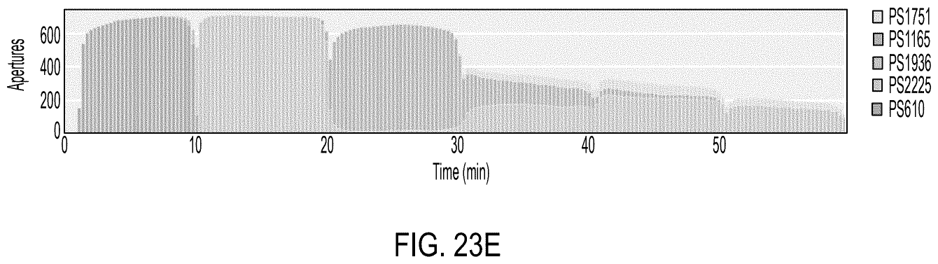

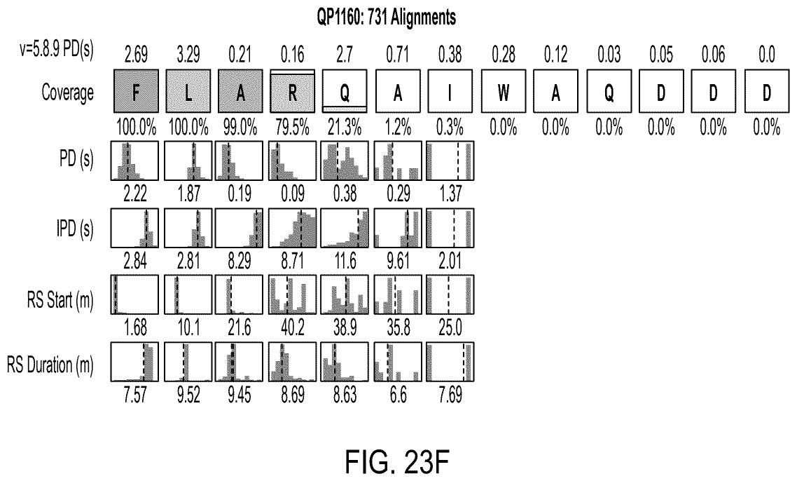

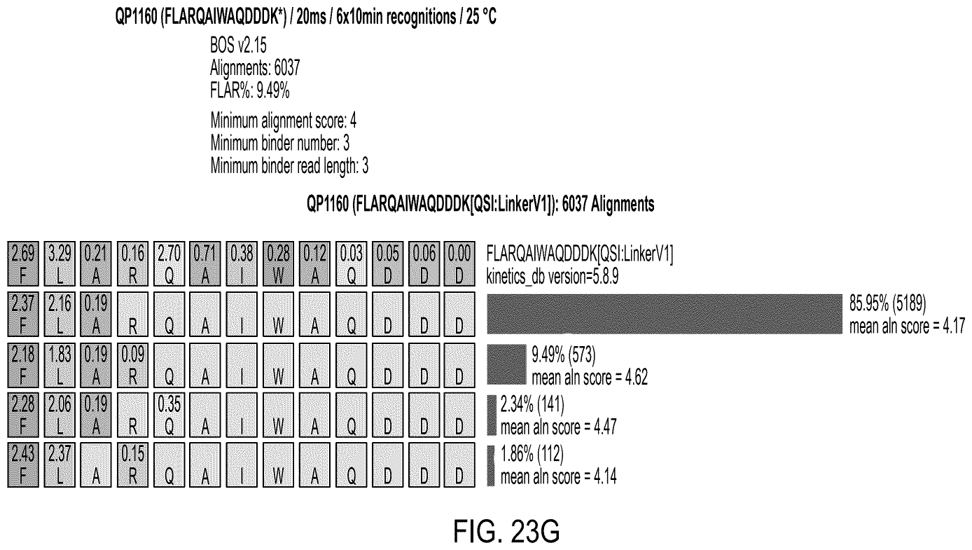

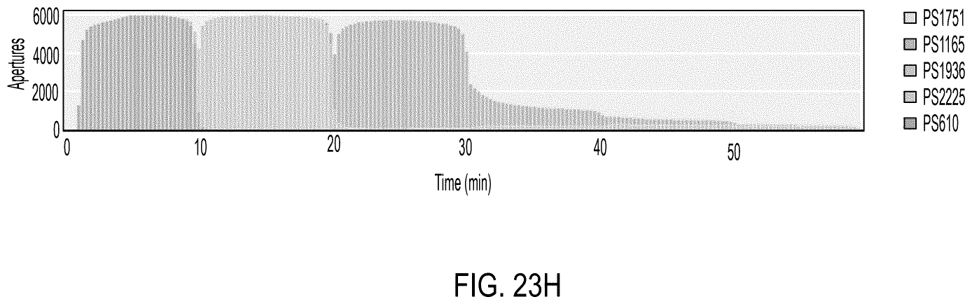

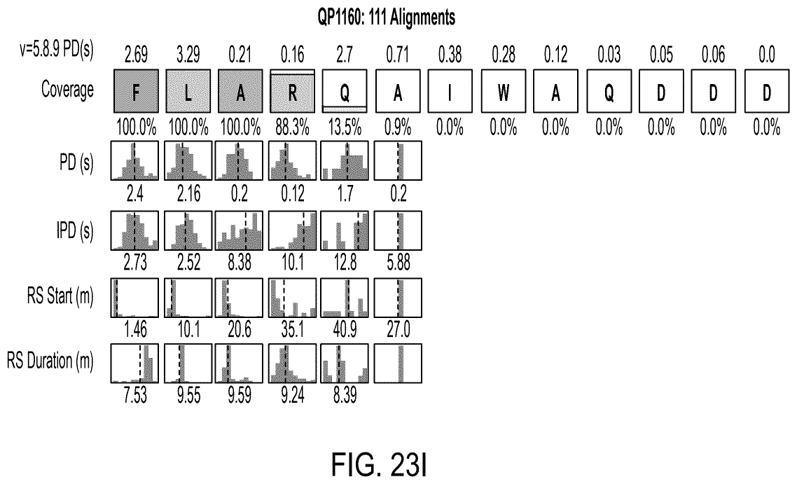

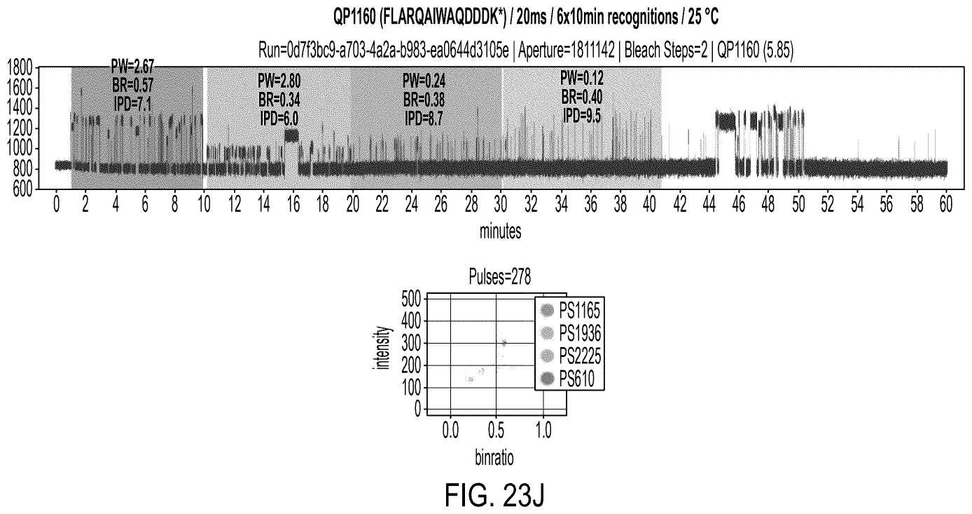

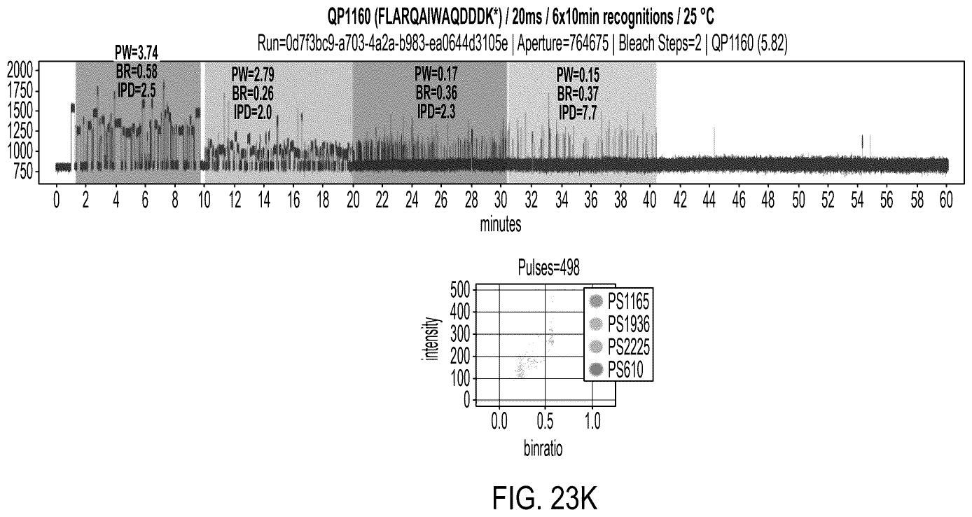

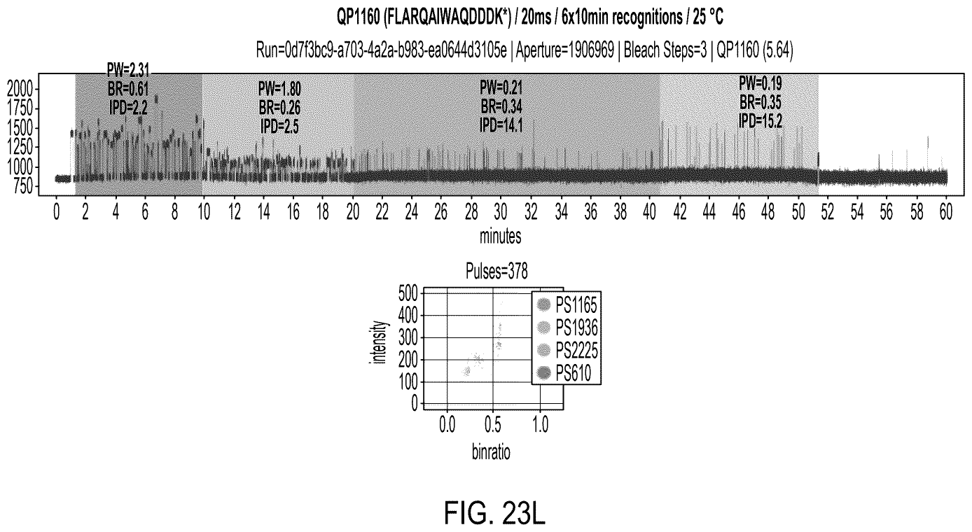

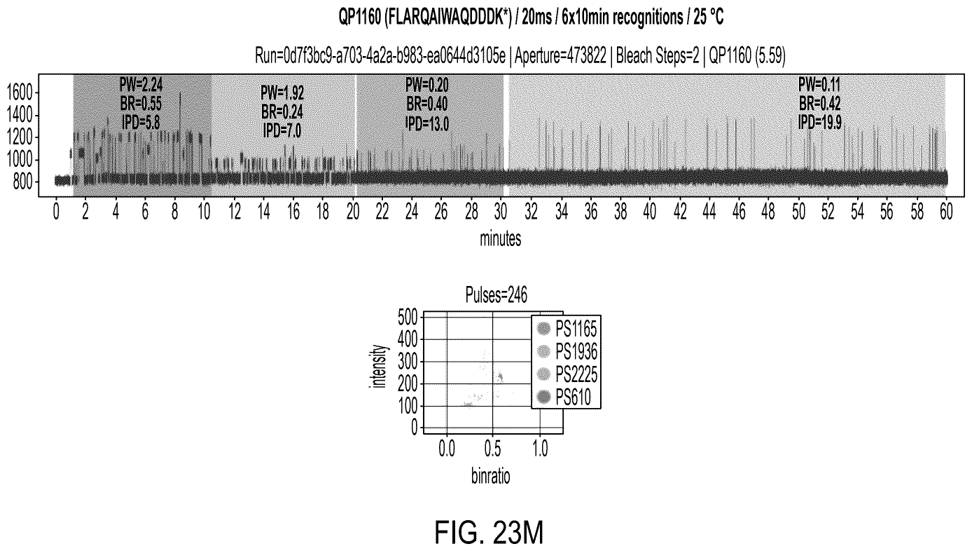

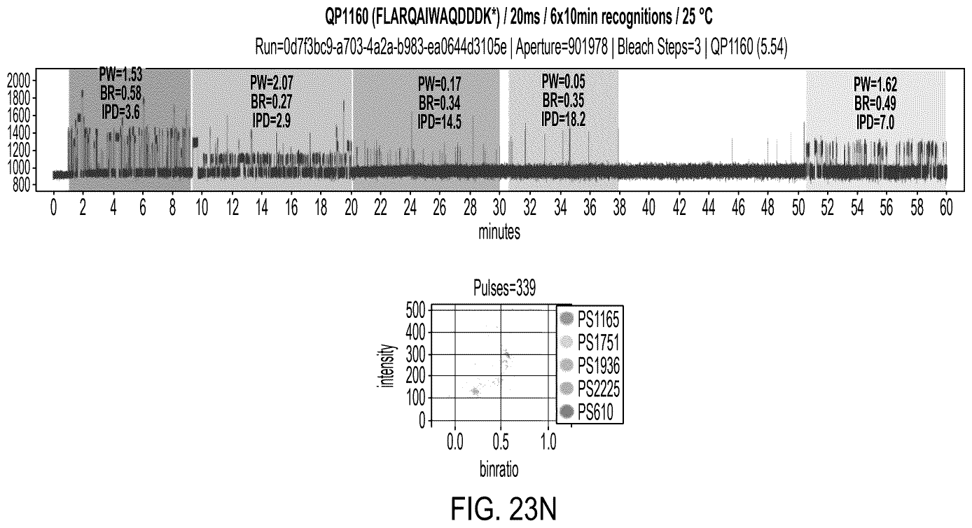

FIGS. 23A-23N provide representative analyses of results (FIGS. 23A-23I) and example signal traces (FIGS. 23J-23N) from sequencing runs involving recognition and controlled cleavage of synthetic peptide QP1160 (FLARQAIWAQDDDK* (SEQ ID NO: 39)). The results from these experiments were analyzed using different parameters of alignment score, binder number, and read length. FIGS. 23A-23I: top panel bar graphs show the % population of the top four sequences; middle panels are apertures showing the pulsing of recognizers for each 10 min recognition of QP1160; and bottom panels show the coverage of each residue and the kinetic information (PD, IPD, RS start, RS duration). Representative signal traces from the sequencing runs are shown in FIGS. 23J-23N.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1.-127. (canceled)

128. A method of polypeptide analysis, the method comprising:
(a) contacting a polypeptide with one or more modification agents to produce a modified first amino acid at a terminus of the polypeptide;
(b) contacting the polypeptide comprising the modified first amino acid with an enzyme, wherein the enzyme removes the modified first amino acid to expose a second amino acid at the terminus of the polypeptide;
(c) contacting the polypeptide having the second amino acid at the terminus with a composition comprising one or more terminal amino acid recognition molecules; and
(d) detecting a series of signal pulses corresponding to binding interactions between the one or more terminal amino acid recognition molecules and the terminus of the polypeptide, wherein the series of signal pulses is indicative of the second amino acid.

129. The method of claim 128, further comprising repeating steps (a)-(d), wherein the repeating results in sequencing a segment of the polypeptide.

130. The method of claim 128, wherein the terminus of the polypeptide is an N-terminus or a C-terminus.

131. The method of claim 128, wherein the polypeptide is immobilized to a surface.

132. The method of claim 128, wherein the one or more modification agents comprise:
(i) an acetylation agent; or
(ii) a handle peptide.

133. The method of claim 128, wherein the one or more modification agents comprise an acetylation agent, and wherein the modified first amino acid is an acetylated first amino acid.

134. The method of claim 133, wherein the acetylation agent comprises one or more succinimidyl acetate compounds, optionally wherein the one or more succinimidyl acetate compounds are selected from the group consisting of an N-hydroxysulfosuccinimide acetate, an N-hydroxysuccinimide acetate, and a sulfosuccinimidyl acetate (Sulfo-NHS acetate).

135. The method of claim 133, wherein the acetylation agent is or comprises a compound of Formula (II): 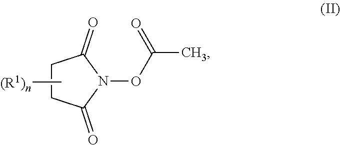
or a salt thereof, wherein:
each instance of R¹ is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two instances of R^{A} are joined together with their intervening atom to form an optionally substituted heterocyclic ring or optionally substituted heteroaryl ring; and
n is 0, 1, 2, 3, or 4.

136. The method of claim 133, wherein the acetylation agent comprises:
(i) an N-terminal acyltransferase, optionally wherein the N-terminal acyltransferase is N-myristoyltransferase or N-alpha-acetyltransferase; and
(ii) an acyl-CoA, optionally wherein the acyl-CoA is acetyl-CoA.

137. The method of claim 133, wherein the enzyme that catalyzes the removal of the acetylated first amino acid comprises an acylpeptide hydrolase.

138. The method of claim 137, wherein the acylpeptide hydrolase comprises:
(i) a wild-type acylpeptide hydrolase or an engineered acylpeptide hydrolase;
(ii) a human acylpeptide hydrolase, Sus scrofa acylpeptide hydrolase, Aeropyrum pernix acylpeptide hydrolase, Bombyx mori acylpeptide hydrolase, or Rattus norvegicus acylpeptide hydrolase; and/or
(iii) an amino acid sequence that is at least 80% identical to the amino acid sequence of any one of SEQ ID NOs: 1-25.

139. The method of claim 128, wherein the one or more modification agents comprise a handle peptide, and wherein the modified first amino acid is a handle peptide-ligated first amino acid.

140. The method of claim 139, wherein the enzyme that catalyzes the removal of the handle peptide-ligated first amino acid is a peptidase.

141. The method of claim 140, wherein:
(i) the handle peptide is one amino acid in length, and the peptidase comprises a dipeptidyl-peptidase; or
(ii) the handle peptide is two amino acids in length, and the peptidase comprises a tripeptidyl-peptidase.

142. The method of claim 128, wherein association of the one or more terminal amino acid recognition molecules with one type of terminal amino acid produces a characteristic pattern in the series of signal pulses that is different from other types of terminal amino acids.

143. The method of claim 128, wherein the method further comprises, prior to (a):
(i) contacting the polypeptide with one or more post-translational modification (PTM)-specific affinity reagents; and
(ii) detecting a series of signal pulses corresponding to binding interactions between the one or more PTM-specific affinity reagents and the polypeptide, wherein the series of signal pulses is indicative of at least one PTM in the polypeptide.

144. A method comprising:
(a) contacting a polypeptide with an acetylation agent under conditions sufficient to produce an acetylated amino acid at a terminus of the polypeptide; and
(b) contacting the polypeptide with an enzyme that catalyzes the removal of the acetylated amino acid from the terminus of the polypeptide.

145. The method of claim 144, wherein the enzyme is an acylpeptide hydrolase comprising an amino acid sequence that is at least 80% identical to the amino acid sequence of any one of SEQ ID NOs: 1-25.

146. A method comprising:
(a) contacting a polypeptide with a handle peptide under ligation conditions sufficient to ligate the handle peptide to an amino acid at a terminus of the polypeptide; and
(b) contacting the polypeptide with an enzyme that catalyzes the removal of the handle peptide-ligated amino acid from the terminus of the polypeptide.

147. An acylpeptide hydrolase comprising an amino acid sequence that is at least 85% identical to SEQ ID NO: 1, wherein the amino acid sequence has one or more substitutions, insertions, and/or deletions relative to SEQ ID NO: 1.


SEQUENCE LISTING (39 sequences)

SEQ ID NO: 1 - protein, 582 aa, Aeropyrum pernix
MRIIMPVEFSRIVRDVERLIAVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 2 - protein, 562 aa, Aeropyrum pernix
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 3 - protein, 582 aa, Aeropyrum pernix
MRIIMPVEFSRIVRAVEALIAVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 4 - protein, 710 aa, Bombyx mori
MSKEFENVIRLYKNFSKVPSIVGAHLSRDTSRVTVKWTVRSTDRGKNTRFVSSYILNESLKVIADCGFGTDISNELLSAVSPNETYRAVIREERNQKDLKKQYLEVWSKNHLAHCIDLNALDLHGDVYADSEFGCLNWSQDESAIVYVAEKKLAKAEPYIVRKPAETPKSDSKETPRKGEEYIYRQDWGEQLVGKHASVIVVCKIESESFTVLDGLPEDWCPGQVRFTLDGKSVIGIAWSTEPRRLGLIFCTNRPGHVFTLSLEDDKALSRLSPEGMSVRAARLSPRGAPVWLQRVARGPHHAGHQLVTGNIAEAPLTVIVDLVQTVTRTDDGRDFHGIYSQSLPARCFSKDGKRIVFSTPQKNEIRSYVVDIESGGMVDVSIRRAAGSTSVLDVHDDVILAAHSSLTSPSQLYVARLPARGSEAEVEWMPVTSQPEVPAGLARSEVTYLQLEHADCADPVKSFSAIYLSPSVQGGSKLPLLVWPHGGPHSNFTNSYSFEAAFFNDLGFAILHVNYRGSTGAGEASMAFLPKRVGDADVKDCKLATETAVARFGLDESKLCLMGGSHGGFLVAHLSGQYPDLFKVVVSRNPVIDVASMFNSTDIADWCAVEAGFPFTEEGPPSEEQLLAMRRCSPLVHAHKVKAPTALMLGSADKRVPHYQGLEYARRLKANGVATRVYLYDDNHSLSSPLVEMDNLINAALWFLKHLDQ

SEQ ID NO: 5 - protein, 732 aa, Sus scrofa
MERQVLLSEPEEAAALYRGLSRQPALSAACLGPEVTTQYGGRYRTVHTEWTQRDLERMENIRFCRQYLVFHDGDSVVFAGPAGNSVETRGELLSRESPSGTMKAVLRKAGGTGTAEEKQFLEVWEKNRKLKSFNLSALEKHGPVYEDDCFGCLSWSHSETHLLYVADKKRPKAESFFQTKALDVTGSDDEMARTKKPDQAIKGDQFLFYEDWGENMVSKSTPVLCVLDIESGNISVLEGVPESVSPGQAFWAPGDTGVVFVGWWHEPFRLGIRFCTNRRSALYYVDLTGGKCELLSDESVAVTSPRLSPDQCRIVYLRFPSLVPHQQCGQLCLYDWYTRVTSVVVDIVPRQLGEDFSGIYCSLLPLGCWSADSQRVVFDSPQRSRQDLFAVDTQMGSVTSLTAGGSGGSWKLLTIDRDLMVVQFSTPSVPPSLKVGFLPPAGKEQAVSWVSLEEAEPFPDISWSIRVLQPPPQQEHVQYAGLDFEAILLQPSNSPEKTQVPMVVMPHGGPHSSFVTAWMLFPAMLCKMGFAVLLVNYRGSTGFGQDSILSLPGNVGHQDVKDVQFAVEQVLQEEHFDAGRVALMGGSHGGFLSCHLIGQYPETYSACVVRNPVINIASMMGSTDIPDWCMVEAGFSYSSDCLPDLSVWAAMLDKSPIKYAPQVKTPLLLMLGQEDRRVPFKQGMEYYRVLKARNVPVRLLLYPKSTHALSEVEVESDSFMNAVLWLCTHLGS

SEQ ID NO: 6 - protein, 732 aa, Rattus norvegicus
MERQVLLSEPQEAAALYRGLSRQPSLSAACLGPEVTTQYGGLYRTVHTEWTQRDLERMENIRFCRQYLVFHDGDSVVFAGPAGNSVETRGELLSRESPSGTMKAVLRKAGGTVSGEEKQFLEVWEKNRKLKSFNLSALEKHGPVYEDDCFGCLSWSHSETHLLYVAEKKRPKAESFFQTKALDISASDDEMARPKKPDQAIKGDQFVFYEDWGETMVSKSIPVLCVLDIDSGNISVLEGVPENVSPGQAFWAPGDTGVVFVGWWHEPFRLGIRYCTNRRSALYYVDLSGGKCELLSDGSLAICSPRLSPDQCRIVYLQYPCLAPHHQCSQLCLYDWYTKVTSVVVDIVPRQLGESFSGIYCSLLPLGCWSADSQRVVFDSAQRSRQDLFAVDTQTGSITSLTAAGSAGSWKLLTIDKDLMVAQFSTPSLPPSLKVGFLPPPGKEQSVSWVSLEEAEPIPGIHWGVRVLHPPPDQENVQYADLDFEAILLQPSNPPDKTQVPMVVMPHGGPHSSFVTAWMLFPAMLCKMGFAVLLVNYRGSTGFGQDSILSLPGNVGHQDVKDVQFAVEQVLQEEHFDARRVALMGGSHGGFLSCHLIGQYPETYSACIARNPVINIASMMGSTDIPDWCMVETGFPYSNSCLPDLNVWEEMLDKSPIKYIPQVKTPVLLMLGQEDRRVPFKQGMEYYRALKARNVPVRLLLYPKSNHALSEVEAESDSFMNAVLWLHTHLGS

SEQ ID NO: 7 - protein, 593 aa, synthetic construct
MRIIMPVEFSRIVRDVERLIAVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERRAAALEHHHHHH

SEQ ID NO: 8 - protein, 573 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERRAAALEHHHHHH

SEQ ID NO: 9 - protein, 593 aa, synthetic construct
MRIIMPVEFSRIVRAVEALIAVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERRAAALEHHHHHH

SEQ ID NO: 10 - protein, 721 aa, synthetic construct
MSKEFENVIRLYKNFSKVPSIVGAHLSRDTSRVTVKWTVRSTDRGKNTRFVSSYILNESLKVIADCGFGTDISNELLSAVSPNETYRAVIREERNQKDLKKQYLEVWSKNHLAHCIDLNALDLHGDVYADSEFGCLNWSQDESAIVYVAEKKLAKAEPYIVRKPAETPKSDSKETPRKGEEYIYRQDWGEQLVGKHASVIVVCKIESESFTVLDGLPEDWCPGQVRFTLDGKSVIGIAWSTEPRRLGLIFCTNRPGHVFTLSLEDDKALSRLSPEGMSVRAARLSPRGAPVWLQRVARGPHHAGHQLVTGNIAEAPLTVIVDLVQTVTRTDDGRDFHGIYSQSLPARCFSKDGKRIVFSTPQKNEIRSYVVDIESGGMVDVSIRRAAGSTSVLDVHDDVILAAHSSLTSPSQLYVARLPARGSEAEVEWMPVTSQPEVPAGLARSEVTYLQLEHADCADPVKSFSAIYLSPSVQGGSKLPLLVWPHGGPHSNFTNSYSFEAAFFNDLGFAILHVNYRGSTGAGEASMAFLPKRVGDADVKDCKLATETAVARFGLDESKLCLMGGSHGGFLVAHLSGQYPDLFKVVVSRNPVIDVASMFNSTDIADWCAVEAGFPFTEEGPPSEEQLLAMRRCSPLVHAHKVKAPTALMLGSADKRVPHYQGLEYARRLKANGVATRVYLYDDNHSLSSPLVEMDNLINAALWFLKHLDQAAALEHHHHHH

SEQ ID NO: 11 - protein, 743 aa, synthetic construct
MERQVLLSEPEEAAALYRGLSRQPALSAACLGPEVTTQYGGRYRTVHTEWTQRDLERMENIRFCRQYLVFHDGDSVVFAGPAGNSVETRGELLSRESPSGTMKAVLRKAGGTGTAEEKQFLEVWEKNRKLKSFNLSALEKHGPVYEDDCFGCLSWSHSETHLLYVAEKKRPKAESFFQTKALDVTGSDDEMARTKKPDQAIKGDQFLFYEDWGENMVSKSTPVLCVLDIESGNISVLEGVPESVSPGQAFWAPGDTGVVFVGWWHEPFRLGIRFCTNRRSALYYVDLTGGKCELLSDESVAVTSPRLSPDQCRIVYLRFPSLVPHQQCGQLCLYDWYTRVTSVVVDIVPRQLGEDFSGIYCSLLPLGCWSADSQRVVFDSPQRSRQDLFAVDTQMGSVTSLTAGGSGGSWKLLTIDRDLMVVQFSTPSVPPSLKVGFLPPAGKEQAVSWVSLEEAEPFPDISWSIRVLQPPPQQEHVQYAGLDFEAILLQPSNSPEKTQVPMVVMPHGGPHSSFVTAWMLFPAMLCKMGFAVLLVNYRGSTGFGQDSILSLPGNVGHQDVKDVQFAVEQVLQEEHFDAGRVALMGGSHGGFLSCHLIGQYPETYSACVVRNPVINIASMMGSTDIPDWCMVEAGFSYSSDCLPDLSVWAAMLDKSPIKYAPQVKTPLLLMLGQEDRRVPFKQGMEYYRVLKARNVPVRLLLYPKSTHALSEVEVESDSFMNAVLWLCTHLGSAAALEHHHHHH

SEQ ID NO: 12 - protein, 743 aa, synthetic construct
MERQVLLSEPQEAAALYRGLSRQPSLSAACLGPEVTTQYGGLYRTVHTEWTQRDLERMENIRFCRQYLVFHDGDSVVFAGPAGNSVETRGELLSRESPSGTMKAVLRKAGGTVSGEEKQFLEVWEKNRKLKSFNLSALEKHGPVYEDDCFGCLSWSHSETHLLYVAEKKRPKAESFFQTKALDISASDDEMARPKKPDQAIKGDQFVFYEDWGETMVSKSIPVLCVLDIDSGNISVLEGVPENVSPGQAFWAPGDTGVVFVGWWHEPFRLGIRYCTNRRSALYYVDLSGGKCELLSDGSLAICSPRLSPDQCRIVYLQYPCLAPHHQCSQLCLYDWYTKVTSVVVDIVPRQLGESFSGIYCSLLPLGCWSADSQRVVFDSAQRSRQDLFAVDTQTGSITSLTAAGSAGSWKLLTIDKDLMVAQFSTPSLPPSLKVGFLPPPGKEQSVSWVSLEEAEPIPGIHWGVRVLHPPPDQENVQYADLDFEAILLQPSNPPDKTQVPMVVMPHGGPHSSFVTAWMLFPAMLCKMGFAVLLVNYRGSTGFGQDSILSLPGNVGHQDVKDVQFAVEQVLQEEHFDARRVALMGGSHGGFLSCHLIGQYPETYSACIARNPVINIASMMGSTDIPDWCMVETGFPYSNSCLPDLNVWEEMLDKSPIKYIPQVKTPVLLMLGQEDRRVPFKQGMEYYRALKARNVPVRLLLYPKSNHALSEVEAESDSFMNAVLWLHTHLGSAAALEHHHHHH

SEQ ID NO: 13 - protein, 568 aa, synthetic construct
MVERLIAVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 14 - protein, 557 aa, synthetic construct
MLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 15 - protein, 553 aa, synthetic construct
MVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 16 - protein, 530 aa, synthetic construct
MGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 17 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAALRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 18 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAALRNVIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 19 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAALRNMIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 20 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAALRNFIEQITGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 21 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMYILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAALRNLIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 22 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMNILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASVVDWEEMYELSDAAMRNLIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 23 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASTVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 24 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASSVDWEEMYELSDAAFRNFIEQLTGGSREIMRSRSPINHVDRIKEPLALIHPQNDSRTPLKPLLRLMGELLARGKTFEAHIIPDAGHAINTMEDAVKILLPAVFFLATQRERR

SEQ ID NO: 25 - protein, 562 aa, synthetic construct
MVEKYSLQGVVDGDKLLVVGFSEGSVNAYLYDGGETVKLNREPINSVLDPHYGVGRVILVRDVSKGAEQHALFKVNTSRPGEEQRLEAVKPMRILSGVDTGEAVVFTGATEDRVALYALDGGGLRELARLPGFGFVSDIRGDLIAGLGFFGGGRVSLFTSNLSSGGLRVFDSGEGSFSSASISPGMKVTAGLETAREARLVTVDPRDGSVEDLELPSKDFSSYRPTAITWLGYLPDGRLAVVARREGRSAVFIDGERVEAPQGNHGRVVLWRGKLVTSHTSLSTPPRIVSLPSGEPLLEGGLPEDLRRSIAGSRLVWVESFDGSRVPTYVLESGRAPTPGPTVVLVHGGPFAEDSDSWDTFAASLAAAGFHVVMPNYRGSTGYGEEWRLKIIGDPCGGELEDVSAAARWARESGLASELYIMGYSYGGYMTLCALTMKPGLFKAGVAGASIVDWEEMYELSDAALRNLIEQLTGGSREIMRSRSPINHVDRIKEPLALIHPQNDSRSPLKPLLRLMGELLARGKTFEAHIIPDAGHAINTMEDAVKILLPAVFFLATQRERR

SEQ ID NO: 26 - protein, 11 aa, synthetic construct
FAAAYPDDDFK

SEQ ID NO: 27 - protein, 11 aa, synthetic construct
  MOD_RES 1: note = acetylated phenylalanine
FAAAYPDDDFK

SEQ ID NO: 28 - protein, 10 aa, synthetic construct
AAAYPDDDFK

SEQ ID NO: 29 - protein, 10 aa, synthetic construct
FLAAYPDDDW

SEQ ID NO: 30 - protein, 10 aa, synthetic construct
  MOD_RES 1: note = acetylated phenylalanine
FLAAYPDDDW

SEQ ID NO: 31 - protein, 9 aa, synthetic construct
LAAYPDDDW

SEQ ID NO: 32 - protein, 13 aa, synthetic construct
FLARQAIWAQDDD

SEQ ID NO: 33 - protein, 13 aa, synthetic construct
  MOD_RES 1: note = acetylated phenylalanine
FLARQAIWAQDDD

SEQ ID NO: 34 - protein, 12 aa, synthetic construct
LARQAIWAQDDD

SEQ ID NO: 35 - protein, 11 aa, synthetic construct
LRQAFAYPDDD

SEQ ID NO: 36 - protein, 10 aa, synthetic construct
RQAFAYPDDD

SEQ ID NO: 37 - protein, 9 aa, synthetic construct
QAFAYPDDD

SEQ ID NO: 38 - protein, 11 aa, synthetic construct
ARQAIWAQDDD

SEQ ID NO: 39 - protein, 14 aa, synthetic construct
FLARQAIWAQDDDK